(19) United States
(12) Patent Application Publication
(10) Pub. No.: US 2025/0154214 A1
(43) Pub. Date: May 15, 2025

(54) TAM RECEPTOR BINDING FUSION MOLECULE HAVING NON-INFLAMMATORY PHAGOCYTOSIS INDUCING ACTIVITY

(71) Applicant: ILLIMIS THERAPEUTICS, INC., Seoul (KR)

(72) Inventors: Chan Hyuk KIM, Daejeon (KR); Won Suk CHUNG, Daejeon (KR); Hyun Cheol JUNG, Daejeon (KR); Se Young LEE, Daejeon (KR)

(73) Assignee: ILLIMIS THERAPEUTICS, INC., Seoul (KR)

(21) Appl. No.: 19/029,866

(22) Filed: Jan. 17, 2025

Related U.S. Application Data
(62) Division of application No. 18/360,984, filed on Jul. 28, 2023.
Application No. 18/360,984 is a continuation-in-part of application No. PCT/KR22/01671, filed on Jan. 28, 2022.

(30) Foreign Application Priority Data
Jan. 29, 2021 (KR) 10-2021-0013045

(51) Int. Cl. C07K 14/47; A61K 38/00; A61P 25/28; C07K 16/18

(52) U.S. Cl. CPC C07K 14/4703; A61P 25/28; C07K 16/18; A61K 38/00; C07K 2317/73; C07K 2319/74

(57) ABSTRACT

A fusion molecule having phagocytosis-inducing activity is disclosed. The fusion molecule contains a first region capable of binding a TAM receptor and a second region capable of binding to a target substance of which aberrant accumulation is associated with or characteristic of diseases. The fusion molecule effectively clears and/or reduces and/or suppresses accumulated abnormal proteins, such as beta-amyloid, tau, alpha-synuclein, huntingtin, or prion, or the like. Uses of the fusion molecule are disclosed. The fusion molecule can be used for prevention or treatment of proteinosis caused by the abnormal accumulation of substances.

### CROSS REFERENCE TO RELATED APPLICATIONS

This application is a Divisional Application of U.S. application Ser. No. 18/360,984 filed Jul. 28, 2023, which is a continuation-in-part of and claims the benefit of PCT Application No. PCT/KR2022/001671 filed Jan. 28, 2022, which claims priority based on Korean Patent Application No. 102021-0013056 filed Jan. 29, 2021, of which the entire contents are incorporated by reference herein.

### INCORPORATION BY REFERENCE OF SEQUENCE LISTING

The content of the electronically submitted sequence listing, file name: Q303799_Sequence_Listing_as_Filed.xml; size: 268.3 KB; and date of creation: Jan. 16, 2025, filed herewith, is incorporated herein by reference in its entirety.

### FILED

The present disclosure relates to fusion molecules that are capable of inducing phagocytosis without inducing inflammatory reaction, their uses, and manufacturing method. The fusion molecules are useful for prevention or treatment of diseases that are caused by or characterized by abnormal accumulation of substances in the body, such as proteopathy. The present disclosure also relates to nucleic acid molecules encoding the fusion molecules. The present disclosure further relates to methods of suppressing abnormal accumulation of substances, promoting clearance of aggregates of substances, and/or treating disorders or diseases that are caused by or characterized by abnormal accumulation of substances, without inducing inflammatory reaction.

### BACKGROUND

Numerous degenerative diseases are characterized by aberrant folding, polymerization and accumulation of proteins. These proteopathies include various types of amyloidosis.

Amyloidosis is a disease in which abnormal proteins called amyloid accumulate in tissues. Amyloid is a protein aggregate that has a diameter of 7-13 nm and a beta-sheet structure and exhibits a fibrous morphology when viewed under a microscope, and it is characterized by being stained with Thioflavin T (ThioT) and Congo red. Amyloid is not normally found in the body, and to date, 36 proteins have been identified as being amyloidogenic (Picken, Acta Haematol. (2020), 143:322-334). Representative examples of amyloidosis diseases include neurological diseases such as Alzheimer's disease, Parkinson's disease, Huntington's disease and prion disease. In addition, there are a number of amyloid diseases having various aspects depending on amyloid-causing proteins and affected organs.

Alzheimer's disease is the biggest cause of dementia and is a fatal disease accompanied by learning and memory impairment. 130 million people are expected to suffer from Alzheimer's disease by 2050 worldwide, and 1 in 9 people among the population above 65 years old have already been diagnosed with Alzheimer's disease.

A hallmark of Alzheimer's disease is that beta-amyloid (Aβ) protein caused by abnormal degradation of amyloid precursor protein (APP), deposits and accumulates around the brain cell membrane. Another hallmark is abnormal hyperphosphorylation of microtubule-associated tau protein.

It has recently been reported that beta-amyloid oligomers and fibrils cause synaptic dysfunction and cytotoxicity through various pathways, and create a vicious cycle that adversely affects nerve cells through functional changes in astrocytes and microglia, which are responsible for immunity in the brain.

Therapeutic drugs for Alzheimer's disease approved by the FDA to date inhibit acetylcholine degradation or inhibit the activity of NMDA receptors, and, thus, provide temporary relief of symptoms, but do not treat the underlying cause or the disease itself. Therefore, there is a need for the development of new treatments for treating Alzheimer's disease and other diseases characterized by aberrant accumulation or deposit of beta-amyloid.

For a therapeutic treatment of Alzheimer's disease, drug development has been conducted for decades with a focus on inhibiting formation of and eliminating beta-amyloid. Unfortunately, however, most of the therapeutic drugs for Alzheimer's disease developed to inhibit formation of and eliminate beta-amyloid failed during the clinical stage due to ineffectiveness or insufficient efficacy. For example, in the case of BACE (β-site amyloid precursor protein cleaving enzyme) inhibitors for reducing beta-amyloid, strategies that prevent additional beta-amyloid production are largely ineffective, because in Alzheimer's patients with cognitive decline, beta-amyloid plaques have already accumulated and neuronal cell death is taking place.

Since the recent studies reporting that monoclonal antibodies which specifically bind to beta-amyloid oligomers and fibrils induced beta-amyloid clearance and restored cognitive function in Alzheimer's disease patients, a strategy to treat Alzheimer's disease through anti-beta-amyloid antibodies has emerged as a new hope.

The mechanisms of action of beta-amyloid monoclonal antibodies proposed to date include inhibition of aggregation of beta-amyloid oligomers and fibrils by binding thereto, or the induction of microglial phagocytosis of beta-amyloid through Fc receptors that recognize the monoclonal antibodies.

However, despite the advances in the development of therapeutic drugs for Alzheimer's disease, current immunotherapy using anti-beta-amyloid monoclonal antibodies shows amyloid-related imaging abnormalities (ARIAs) accompanied by severe edema in 55% of patients treated with the antibodies, and for this reason, about 35% of the ARIA patients were dropped from clinical trials. The ARIA phenomenon is known to be due to synaptotoxicity and cytotoxicity caused by inflammatory responses that are inevitably activated when anti-beta-amyloid monoclonal antibodies stimulate Fc receptors of microglia cells.

Since synapses and neurons in the brain respond sensitively to inflammatory cytokines, treatment using anti-beta-amyloid monoclonal antibodies has an inherent problem in that it inevitably causes damages to neurons and synapses, even if it clears beta-amyloid to some extent. In addition to monoclonal antibodies, companies such as Alector and Denali presented strategies to improve the microglia's ability to clear beta-amyloid by activating targets such as TREM2 that regulate the immunological mechanism of microglia, and these strategies have received a lot of attention. However, even in these strategies, when microglia are excessively activated, synaptic damage due to an increase in overall phagocytotic capacity is expected.

Therefore, an important task in the treatment of Alzheimer's disease is to develop therapeutic modalities to selectively clear only beta-amyloid oligomers and fibrils without causing inflammatory responses and synaptic damage, and these drugs are expected to make a significant contribution to the treatment of Alzheimer's disease.

Furthermore, there is a need to selectively clear only a substance of which abnormal accumulation causes disorders or accumulates thereof as a target, for example, abnormally accumulated proteins causing proteopathy, without causing inflammatory responses and consequent additional tissue damage reported in conventional experimental drugs. The present disclosure meets this need by providing therapeutic modalities for selectively clearing abnormally accumulated proteins that cause or characterize certain disease.

### SUMMARY

The present disclosure relates to fusion molecules having phagocytosis-inducing activity without inducing inflammatory responses. One aspect of the present disclosure provides a fusion molecule having phagocytosis-inducing activity, the fusion molecule comprising: a first region that is capable of binding to a TAM (Tyro3, Axl and MerTK) receptor; and a second region that specifically binds to a target substance to be cleared or decreased, and the fusion molecule does not induce inflammatory responses. In embodiments, the fusion molecule does not have an effector function and does not induce Fc-mediated inflammatory responses.

In some embodiments, the TAM receptor may be any one selected from the group consisting of Tyro3, Axl, MerTK, or a combination thereof, which are capable of inducing phagocytosis by binding to a laminin G-like domain (or LG domain) of a phagocytic cell including, but not limited to, macrophages or microglial cells. In embodiments, the TAM receptor may be Axl receptor.

In embodiments, the first region may comprise Gas6, ProS1, Tubby, Tulp1, Gal3, or an active fragment thereof, which each is capable of specifically binding to a TAM receptor. The first region may be selected from Gas6, ProS1, or an active fragment thereof, which each is capable of specifically binding to a TAM receptor. In embodiments, the first region may comprise or consist essentially of Gas6 or an active fragment thereof that is capable of binding to TAM receptor. In embodiments, the first region comprising or consisting essentially of Gas6 or an active fragment thereof is capable of binding to Axl receptor.

In certain embodiments, the first region may comprise a laminin G-like domain of Gas6 or ProS1, or an active fragment thereof, which contains a laminin G-like domain as a phagocytosis-related bridging molecule which is abundantly expressed in various tissue, and thus is able to induce phagocytosis through a TAM receptor. In embodiments, the laminin G-like domain may comprise an LG1 domain, an LG2 domain, or a combination thereof, and may preferably include both an LG1 domain and an LG2 domain, which are able to induce phagocytosis by binding to the TAM receptor.

Exemplary embodiments are directed to a binding molecule or fusion molecule comprising a first region capable of binding to a TAM receptor and a second region capable of specifically binding to a target substance, said target substance being a substance of which aberrant accumulation in a living tissue is characteristic of or associated with a disease, wherein the first region and the second region are coupled to each other directly or via a linker, wherein the first region comprises
(a) a TAM receptor ligand;
(b) an anti-Axl antibody or an antigen-binding fragment thereof;
(c) an anti-Tyro3 antibody or an antigen-binding fragment thereof; or
(d) an anti-MerTK antibody or an antigen-binding fragment thereof, with proviso that when the first region comprises an anti-MerTK antibody or an antigen-binding fragment thereof, the molecule is not a bispecific antibody; or
(e) combinations thereof.

According to some embodiments, the binding molecule may further comprise a scaffold bound to the first region, to the second, or to both of the first region and the second region at different positions.

In embodiments, the first region is a TAM receptor ligand and the TAM receptor ligand comprises a sequence selected from the group consisting of SEQ ID NOS: 1-113 or a sequence having at least 85% of sequence identity thereto.

In still some embodiments, the first region is capable of binding to an Axl receptor the first region capable of binding to an Axl receptor comprises one or more sequences selected from the group consisting of SEQ ID NO: 1, SEQ ID NO: 2, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, and SEQ ID NO: 87, or a sequence having at least 85% of sequence identity thereto.

In still some embodiments, the first region is capable of binding to an Axl receptor the first region capable of binding to an Axl receptor comprises the sequence of SEQ ID NO: 1 or a sequence having at least 85% of sequence identity thereto, and/or the sequence of SEQ ID NO: 2 or a sequence having at least 85% of sequence identity thereto.

In still another embodiment, the first region capable of binding to an Axl receptor the first region capable of binding to an Axl receptor comprises the sequence of SEQ ID NO: 5 or a sequence having at least 85% of sequence identity thereto.

In still some embodiments, the first region is capable of binding to an Axl receptor the first region capable of binding to an Axl receptor comprises one or more sequences selected from the group consisting of SEQ ID NO: 3, SEQ ID NO: 4, SEQ ID NO: 6, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, and SEQ ID NO: 113, or a sequence having at least 85% of sequence identity thereto.

In still some embodiments, the first region is capable of binding to an Axl receptor the first region capable of binding to an Axl receptor comprises the sequence of SEQ ID NO: 3 or a sequence having at least 85% of sequence identity thereto, and/or the sequence of SEQ ID NO: 4 or a sequence having at least 85% of sequence identity thereto.

In still another embodiment, the first region capable of binding to an Axl receptor the first region capable of binding to an Axl receptor comprises the sequence of SEQ ID NO: 6 or a sequence having at least 85% of sequence identity thereto.

In embodiments, the fusion molecule (or binding molecule) may comprise the first region comprising the sequence of SEQ ID NO: 1, SEQ ID NO: 2, or a combination thereof, or a sequence having at least 85% sequence identity thereto. In an embodiment, the combination of the sequence of SEQ ID NO: 1 and SEQ ID NO: 2 may comprise the sequence of SEQ ID NO: 5 or a sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to SEQ ID NO: 5.

In embodiments, the fusion molecule (or binding molecule) may comprise the first region comprising the sequence of SEQ ID NO: 3, SEQ ID NO: 4, or a combination thereof, or a sequence having at least 85% sequence identity thereto. In an embodiment, the combination of the sequence of SEQ ID NO: 3 and SEQ ID NO: 4 may comprise the sequence of SEQ ID NO: 6 or a sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to SEQ ID NO: 6.

In embodiments, the fusion molecule (or binding molecule) may comprise the sequence of amino acid residues 31-871 of SEQ ID NO: 136, amino acid residues 31-687 of SEQ ID NO:138, amino acid residues 31-697 of SEQ ID NO:140, amino acid residues 31-684 of SEQ ID NO: 150, amino acid residues 31-676 of SEQ ID NO: 152, amino acid residues 25-673 of SEQ ID NO: 154, amino acid residues 22-662 of SEQ ID NO: 156, or amino acid residues 22-885 of SEQ ID NO: 158, amino acid residues 22-908 of SEQ ID NO: 162, amino acid residues 22-919 of SEQ ID NO: 163, amino acid residues 22-919 of SEQ ID NO: 165, or a sequence having at least 90% sequence identity thereto (wherein a different linker can be used in place the linkers as shown in Tables 3 and 5-10.

In embodiments, the first region comprising a laminin G-like domain of Gas6 or ProS1, or an active fragment thereof, does not comprise a Gla domain. Without being bound to a particular theory, it is expected that the lacking of Gla domain in the first region may make the fusion molecule not be able to recognize phosphatidylserine (PS) of TAM receptor, while the second region is able to induce phagocytosis by recognizing a target substance.

In some embodiments, the first region comprising a laminin G-like domain of Gas6 or ProS1, or an active fragment thereof, does not comprise a Gla domain and does not comprise an EGF domain. The lacking of EGF domain in the first region provides an advantage in the manufacturing process of the fusion molecule to increase the yield by suppressing an aggregation of the fusion molecule during the purification step. In some embodiments, the fusion molecule (or binding molecule) may form a homomultimer or a heteromultimer, or form a linear multimer as a single chain.

According to embodiments, the target substance that is to be cleared or decreased and to which the second region specifically binds, may be a substance that accumulates in living tissue, causing a disease. For example, it may be a substance accumulated in an affected (i.e., diseased) tissue of a patient or circulating in blood of a patient. The substance may be protein. That is, the disease may be, but not limited thereto, proteopathy. In certain embodiments, the target substance may be amyloid. That is, the proteopathy may be amyloidosis. The target substance may be one or more of the amyloid substances listed in Table 1 below or APOE or apoptosis-associated spec-like protein containing a caspase activating recruitment domain (ASC-speck), of which abnormal accumulation or deposit is associated with or characteristic of a disease, and in this case, the disease may be a disease in which each abnormally accumulated substance is detected. For example, the proteopathy may be Alzheimer's disease, Parkinson's disease, Huntington's disease, and Prion disease, and in this case, target substances may be β-amyloid, tau, α-synuclein, huntingtin, and prion proteins, respectively, which are identified as proteins of which abnormal accumulation causes the diseases. Aberrant accumulation of APOE is associated with Alzheimer's disease, cerebral amyloid angiopathy, and/or cardiovascular disease. Aberrant accumulation of apoptosis-associated spec-like protein containing a caspase activating recruitment domain (ASC-speck) is associated with Alzheimer's Disease, Parkinsons's Disease, Huntington's disease, Multiple System Atrophy, Amyotrophic Lateral Sclerosis, Sinocerebellar ataxia. Frontotemporal Dementia, Frontotemporal Lobar Degeneration, Mild Cognitive Impairment, Parkinson-plus syndromes, Pick disease, Progressive isolated aphasia, Grey-matter degeneration [Alpers], Subacute necrotizing encephalopathy, and Lewy body dementia.

TABLE 1

| | | Diseases associated with or |
|---|---|---|
| | | characterized by aberrant |
| Target Substance | Abbreviation | accumulation of target substance |
| β-Amyloid | Aβ | Alzheimer's disease, Hereditary cerebral |
| | | haemorrhage with amyloidosis, etc. |
| Amyloid precursor | Aβ | Alzheimer's disease, Hereditary cerebral |
| protein-derived | | haemorrhage with amyloidosis, etc. |
| β-amyloid | | |
| α-Synuclein | α-Syn | Parkinson's disease, Parkinson's |
| | | dementia, dementia with Lewy bodies, |
| | | multiple system atrophy, etc. |
| Prp^{Sc} | PrP | Transmissible spongiform |
| | | encephalopathy (fatal familial insomnia, |
| | | Gerstmann-Straussler-Scheinker disease, |
| | | Creutzfeldt-Jacob disease, new variant |
| | | Creutzfeldt-Jacob disease, etc.), etc. |
| Microtubule- | Tau | Tauopathies (Pick's disease, progressive |
| associated | | supranuclear palsy, corticobasal |
| protein tau | | degeneration, frontotemporal dementia |
| | | with parkinsonism linked to chromosome |
| | | 17, argyrophilic grain disease, etc.), |
| | | Alzheimer's disease, Parkinson's disease, |
| | | etc. |
| Huntingtin | HTT exon 1 | Huntington's disease, etc. |
| exon 1 | | |
| TAR DNA-binding | TDP43 | Frontotemporal dementia, amyotrophic |
| protein 43 | | lateral sclerosis (ALS), etc. |
| Superoxide | SOD1 | Amyotrophic lateral sclerosis (ALS), etc. |
| dismutase 1 | | |
| ABri peptide | bri | Familial British dementia |
| ADan peptide | dan | Familial Danish dementia |
| Immunoglobulin | AL | Light-chain amyloidosis |
| light-chain fragment | | |
| Immunoglobulin | AH | Heavy-chain amyloidosis |
| heavy-chain fragment | | |
| N-terminal fragment of | AA | AA amyloidosis |
| serum amyloid A protein | | |
| Transthyretin | ATTR | Senile systemic amyloidosis, familial |
| | | amyloid polyneuropathy, familial |
| | | amyloid cardiomyopathy, leptomeningeal |
| | | amyloidosis |
| β-2 microglobulin | Aβ2M | Dialysis-related amyloidosis, hereditary |
| | | visceral amyloidosis |
| N-terminal fragment of | AApoAI | ApoAI amyloidosis |
| apolipoprotein AI | | |
| C-terminally extended | AApoAII | ApoAII amyloidosis |
| apolipoprotein AII | | |
| N-terminal fragment of | AApoAIV | ApoAIV amyloidosis |
| apolipoprotein AIV | | |
| apolipoprotein C-II | AApoCII | ApoCII amyloidosis |
| apolipoprotein C-III | AApoCIII | ApoCIII amyloidosis |
| Gelsoliin fragment | AGel | Familial amyloidosis, Finnish type, |
| | | Hereditary gelsolin amyloidosis |
| Lysozyme | ALys | Hereditary non-neuropathic systemic |
| | | amyloidosis |
| Fibrinogen alpha | AFib | Fibrinogen amyloidosis |
| chain fragment | | |
| N-terminally | ACys | Hereditary cerebral hemorrhage with |
| truncated cystatin C | | amyloidosis, Icelandic type |
| Amylin, IAPP | IAPP | Diabetes mellitus type 2, insulinoma |
| Calcitonin | Cal | Medullary carcinoma of the thyroid |
| Atrial natriuretic | AANF | Cardiac arrhythmias, isolated atrial |
| factor | | amyloidosis |
| Prolactin | PRL | Pituitary prolactinoma |
| Insulin | AIns | Localized amyloidosis at insulin injection |
| | | sites |
| Lactadherin or medin | AMed | Aortic medial amyloidosis |
| Lactotransferrin | LTF | Gelatinous drop-like corneal dystrophy |
| or lactoferrin | | |
| Odontogenic ameloblast- | ODAM | Calcifying epithelial odontogenic tumors |
| associated protein | | |
| pulmonary surfactant- | SPC | Pulmonary alveolar proteinosis |
| associated protein C | | |
| Leukocyte cell-derived | ALECT2 | Renal LECT2 amyloidosis |
| chemotaxin-2 | | |
| Galectin-7 | Agal7 | Lichen amyloidosis, macular amyloidosis |
| Corneodesmosin | Cor | Hypotrichosis simplex of the scalp |
| C-terminal fragment | Ker | Lattice corneal dystrophy; type I, 3A or |
| of TGFBI (or | | Avellino |
| keratoepithelin) | | |
| SGI (Semenogelin-1) | ASem1 | Seminal vesicle amyloidosis |
| S100 protein (A8 or A9) | (no abbreviation) | Prostate cancer |
| Enfuvirtide | AEnf | Injection-localized amyloidosis |
| Apolipoprotein E | APOE | Alzheimer's disease, cerebral amyloid |
| | | angiopathy, cardiovascular disease |
| Apoptosis-associated | ASC | Alzheimer's Disease, Parkinsons's |
| Spec-like protein | | Disease, Huntington's disease, Multiple |
| containing a Caspase | | System Atrophy, Amyotrophic Lateral |
| Activating | | Sclerosis, Sinocerebellar ataxia. |
| Recruitment Domain | | Frontotemporal Dementia, |
| | | Frontotemporal Lobar Degeneration, |
| | | Mild Cognitive Impairment, Parkinson- |
| | | plus syndromes, Pick disease, |
| | | Progressive isolated aphasia, Grey-matter |
| | | degeneration [Alpers], Subacute |
| | | necrotizing encephalopathy, and Lewy |
| | | body dementia |

In embodiments, the present disclosure is directed to a nucleic acid or polynucleotide encoding the fusion proteins described above.

In embodiments, the present disclosure is directed to a vector containing the nucleic acid or polynucleotide.

Embodiments are directed to a host cell containing the vector.

Another aspect of the present disclosure provides a method of producing a therapeutic fusion molecule for treatment of a disease or disorder associated with or characterized by aberrant accumulation of substance in a subject, comprising expressing the fusion molecule by culturing a host cell under a condition for expressing the fusion molecule.

In embodiments, the present disclosure is directed to a method of reducing or enhancing a reduction of aberrant deposit of substance that causes or characterizes certain disorder or diseases in a subject, which method comprises administering to the subject an effective amount of a fusion molecule or a polynucleotide encoding the fusion molecule, wherein the fusion molecule comprises a first region that is capable of binding to a TAM (Tyro3, Axl and MerTK) receptor on surface of a cell in the subject; and a second region that specifically binds to the substance. In non-limiting embodiments, the substance and the disease or disorder may be one or more of those listed in Table 1. In non-limiting embodiments, the fusion molecule does not have an effector function and does not induce Fc-mediated inflammatory responses. For example, the fusion molecule does not comprise a moiety to bind to an Fc receptor, and preferably may comprise an Fc region variant that does not bind to an Fc receptor (particularly an Fcγ receptor).

In embodiments, the present disclosure is directed to a method of removing or clearing or enhancing clearance of aberrant deposit of substance that causes or characterizes certain disorder or diseases in a subject, which method comprises administering to the subject an effective amount of a fusion molecule or a polynucleotide encoding the fusion molecule, wherein the fusion molecule comprises a first region that is capable of binding to a TAM (Tyro3, Axl and MerTK) receptor on surface of a cell in the subject; and a second region that specifically binds to the substance. In non-limiting embodiments, the substance and the disease or disorder may be one or more of those listed in Table 1. In non-limiting embodiments, the fusion molecule does not have an effector function and does not induce inflammatory responses. For example, the fusion molecule does not comprise a moiety to bind to an Fc receptor, and may comprise an Fc region variant that does not bind to an Fc receptor (particularly an Fcγ receptor).

In embodiments, the present disclosure is directed to a method of suppressing formation of aberrant accumulations of substance in a subject. The method comprises administering to the subject an effective amount of a fusion molecule or a polynucleotide encoding the fusion molecule, wherein the fusion molecule comprises a first region that is capable of binding to a TAM (Tyro3, Axl and MerTK) receptor on surface of a cell in the subject and a second region that specifically binds to the substance. In non-limiting embodiments, the substance and the disease or disorder may be one or more of those listed in Table 1. In non-limiting embodiments, the fusion molecule does not have an effector function and does not induce Fc-mediated inflammatory responses. For example, the fusion molecule does not comprise a moiety to bind to an Fc receptor, and preferably may comprise an Fc region variant that does not bind to an Fc receptor (particularly an Fcγ receptor).

In embodiments, the present disclosure is directed to a method of treating or preventing a disorder or disease in a subject, wherein the disorder or disease is characterized by or caused by aberrant accumulation of substance. The method comprises administering to the subject an effective amount of a fusion molecule or a polynucleotide encoding the fusion molecule, wherein the fusion molecule comprises a first region that is capable of binding to a TAM (Tyro3, Axl and MerTK) receptor on surface of a cell in the subject; and a second region that specifically binds to the substance. In non-limiting embodiments, the substance and the disease or disorder may be one or more of those listed in Table 1. In non-limiting embodiments, the fusion molecule does not have an effector function and does not induce Fc-mediated inflammatory responses. For example, the fusion molecule does not comprise a moiety to bind to an Fc receptor, and preferably may comprise an Fc region variant that does not bind to an Fc receptor (particularly an Fcγ receptor).

In embodiments, the present disclosure is directed to a method of delaying development of a symptom associated with a disease that is characterized by, associated with, or caused by aberrant accumulation of substance, in a subject. The method comprises administering to the subject an effective amount of a fusion molecule or a polynucleotide encoding the fusion molecule, a vector comprising the polynucleotide, wherein the fusion molecule comprises a first region that is capable of binding to a TAM (Tyro3, Axl and MerTK) receptor on surface of a cell in the subject; and a second region that specifically binds to the substance. In non-limiting embodiments, the substance and the disease or disorder may be one or more of those listed in Table 1. In non-limiting embodiments, the fusion molecule does not have an effector function and does not induce Fc-mediated inflammatory responses. For example, the fusion molecule does not comprise a moiety to bind to an Fc receptor, and preferably may comprise an Fc region variant that does not bind to an Fc receptor (particularly an Fc receptor).

In embodiments, the present disclosure provides a method of reducing a substance of which aberrant accumulation is associated with or characteristic of a disease or disorder, in a subject. The method comprises administering to the subject an effective amount of a fusion molecule or a polynucleotide encoding the fusion molecule, wherein the fusion molecule comprises a first region that is capable of binding to a TAM (Tyro3, Axl and MerTK) receptor on surface of a cell in the subject, and a second region that specifically binds to the substance. The substance may be soluble, oligomeric, or aggregated form. In some embodiments, the toxic effects of accumulated substance are inhibited and/or reduced. Thus, the method of the disclosure can be used to treat any disease in which accumulation of a substance is present or suspected. In non-limiting embodiments, the substance and the disease or disorder may be one or more of those listed in Table 1. In non-limiting embodiments, the fusion molecule does not have an effector function and does not induce Fc-mediated inflammatory responses. For example, the fusion molecule does not comprise a moiety to bind to an Fc receptor, and preferably may comprise an Fc region variant that does not bind to an Fc receptor (particularly an Fc receptor).

In the above methods, according to embodiments thereof, the aberrant deposits of substance are in the brain (brain tissue) of the subject. In some embodiments, the aberrant deposits of substance are in the cerebral vasculature. In some embodiments, the aberrant accumulation of substance is in the circulatory system. In some embodiments, the aberrant accumulation of substance is in various tissues such as heart, kidney, liver, and the like.

In embodiments, the present disclosure is directed to a pharmaceutical composition comprising an effective amount of any of the above-disclosed fusion molecule or polynucleotides encoding the fusion molecule, and a pharmaceutical acceptable excipient. In non-limiting embodiments, the fusion molecule does not have an effector function and does not induce Fc-mediated inflammatory responses. For example, the fusion molecule does not comprise a moiety to bind to an Fc receptor, and preferably may comprise an Fc region variant that does not bind to an Fc receptor (particularly an Fcγ receptor).

In embodiments, the present disclosure is directed to kits comprising an effective amount of any of the above-disclosed fusion molecule or polynucleotides encoding the fusion molecule. The kits are generally in suitable packaging and provided with appropriate instructions, are useful for any of the methods described herein.

These and other aspects, objects, features and advantages of the example embodiments will become apparent to those having ordinary skill in the art upon consideration of the following detailed description of illustrated example embodiments.

### BRIEF DESCRIPTION OF DRAWINGS

FIG. 1A schematically illustrates the structure of TAM receptors and TAM ligands. In the structure of TAM receptors: the N-terminal starts with 2 Ig-like domains, followed by 2 fibronectin type 3 domains, followed by a single-pass transmembrane domain and a protein tyrosine kinase at the C-terminal. In the structure of the TAM ligands protein S (Pros1) and Gas6, the N-terminal contains a GLA domain, followed by a thrombin-sensitive region (TSR), followed by 4 EGF-like domains, followed by a C-terminal (SHBG-like domain, consisting of 2 LG repeats. In FIG. 1A, EGF stands for epidermal growth factor, Ig stands for immunoglobulin, LG stands for laminin G, and SHBG stands for sex hormone-binding globulin.

FIG. 1B schematically shows beta-amyloid- and FITC-binding phagocytosis inducing fusion molecule comprising Gas6 as a non-limiting example of first region of the fusion molecule.

FIG. 1C through FIG. 1M schematically show non-limiting exemplary embodiments of the structure of the fusion molecules.

FIG. 2 shows the results of Western blot analysis of a non-limiting exemplary chimeric phagocytosis inducer comprising a FLAG Tag, produced according to Preparation Example 1.

FIG. 3 schematically shows an action of a non-limiting exemplary chimeric phagocytosis inducer, produced according to Preparation Example 1, on TAM receptor.

FIG. 4 shows the evaluation results for selective beta-amyloid clearing ability of αAβ-Gas6.

FIG. 5 shows the evaluation results for beta-amyloid clearing ability of αAβ-Gas6 in the HMC3 cell line by beta-amyloid engulfment assay in vitro.

FIG. 6 shows the evaluation results for beta-amyloid clearing ability of αAβ-Gas6 in the HMC3 cell line by beta-amyloid engulfment assay in vitro.

FIG. 7 shows results indicating that the beta-amyloid clearing ability of αAβ-Gas6 is associated with or dependent on Gas6 binding to Axl among TAM receptors.

FIG. 8 shows results indicating that the beta-amyloid clearing ability of αAβ-Gas6 is associated with or dependent on Gas6 binding to Axl among TAM receptors.

FIG. 9 shows results indicating that the beta-amyloid clearing ability of αAβ-Gas6 is associated with or dependent on Gas6 binding to Axl among TAM receptors.

FIG. 10 shows the results of comparative analysis of the activation of inflammatory response signaling by αAβ-Gas6 and aducanumab using THP-Axl cells.

FIG. 11 shows the results of comparative analysis of the levels of pro-inflammatory cytokine secretion by αAβ-Gas6 and aducanumab using THP-Axl cells.

FIG. 12 shows the evaluation results for anti-inflammatory activity of αAβ-Gas6.

FIG. 13 shows results indicating that the beta-amyloid clearing ability of microglia was significantly increased by αAβ-Gas6.

FIG. 14 shows results indicating that the beta-amyloid clearing ability of astrocytes was significantly increased by αAβ-Gas6.

FIG. 15 shows results indicating that the transcriptional levels of pro-inflammatory cytokines in astrocytes were changed by αAβ-Gas6 and aducanumab.

FIG. 16 shows results indicating that the transcriptional levels of pro-inflammatory cytokines in BV2 were changed by αAβ-Gas6 and aducanumab.

FIG. 17 shows the evaluation results for beta-amyloid plaque clearing ability of αAβ-Gas6 through administration of αAβ-Gas6 protein in 5XFAD Alzheimer's disease model mice.

FIG. 18 shows the evaluation results for beta-amyloid plaque clearing ability of αAβ-Gas6 through administration of αAβ-Gas6 virus in 5XFAD Alzheimer's disease model mice.

FIG. 19 shows results indicating that beta-amyloid contained in lysosomes were increased by microglia-mediated clearance in 5XFAD Alzheimer's disease model mice upon administration of αAβ-Gas6 protein.

FIG. 20 shows results indicating that beta-amyloid contained in lysosomes were increased by astrocyte-mediated clearance in 5XFAD Alzheimer's disease model mice upon administration of αAβ-Gas6 protein.

FIG. 21 shows results indicating that beta-amyloid contained in lysosomes were increased by microglia-mediated clearance in 5XFAD Alzheimer's disease model mice upon administration of αAβ-Gas6 virus.

FIG. 22 shows results indicating that beta-amyloid contained in lysosomes were increased by astrocyte-mediated clearance in 5XFAD Alzheimer's disease model mice upon administration of αAβ-Gas6 virus.

FIG. 23 shows results indicating that microglia-mediated synapse engulfment that abnormally increased in 5XFAD Alzheimer's disease model mice due to the side effect of aducanumab was significantly restored upon administration of αAβ-Gas6 virus.

FIG. 24 shows results indicating that microglia-mediated synapse engulfment that abnormally increased in 5XFAD Alzheimer's disease model mice due to the side effect of aducanumab was significantly restored upon administration of αAβ-Gas6 virus.

FIG. 25 shows an experimental protocol for evaluating cognitive and memory abilities in 5XFAD Alzheimer's disease model mice upon administration of αAβ-Gas6 virus.

FIG. 26 shows results indicating that cognitive and memory abilities in 5XFAD Alzheimer's disease model mice were more restored upon administration of αAβ-Gas6 virus than administration of aducanumab.

FIG. 27 shows the evaluation results for tau clearing ability of αTau-Gas6 in the HMC3 cell line by in vitro tau engulfment assay.

FIG. 28 shows the evaluation results for alpha-synuclein clearing ability of αTau-Gas6 in the HMC3 cell line by in vitro tau engulfment assay.

FIG. 29 shows the evaluation results for beta-amyloid clearing ability of αAβ-ProS1 in primary-cultured astrocytes by in vitro tau engulfment assay.

FIG. 30 shows the evaluation results for beta-amyloid clearing ability of αAβ(Fab)-Gas6 in the HMC3 cell line by in vitro tau engulfment assay.

FIG. 31 shows the evaluation results for beta-amyloid clearing ability of αAβ(Fab)-Gas6 in the HMC3 cell line by in vitro tau engulfment assay.

### DETAILED DESCRIPTION

Methods and compositions are provided for reducing or suppressing formation of or clearing a target substance of which accumulation is associated with or characteristic of a disorder or disease via a phagocytosis, preventing or treating an individual having a disease or disorder characterized by an aberrant accumulation of a substance, improving symptoms of a disease or disorder characterized by an aberrant accumulation of a substance, and/or a target substance of which accumulation is associated with or characteristic of a disorder or disease via a phagocytosis.

Where a range of values is provided, it is understood that each intervening value, to the tenth of the unit of the lower limit unless the context dearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

### Definitions

As used herein, the singular forms “a,” “an,” and “the,” refer to both the singular as well as plural, unless the context clearly indicates otherwise. Therefore, for example, reference to “a cell” includes a plurality of such cells and reference to “the peptide” includes reference to one or more peptides and equivalents thereof, e.g. polypeptides, known to those skilled in the art, and so forth.

As used herein, the terms “about” and “consisting essentially of” refers to a value or composition that is within an acceptable error range for the particular value or composition as determined by one of ordinary skill in the art, which will depend in part on how the value or composition is measured or determined, i.e., the limitations of the measurement system. For example, “about” or “consisting essentially of” can mean within 1 or more than 1 standard deviation per the practice in the art. Alternatively, “about” or “consisting essentially of” can mean a range of up to 10% (i.e., ±10%). For example, “about 5 mg” can include any number between 4.5 mg and 5.5 mg (for 10%), between 4.75 mg and 6.25 mg (for 5%), between 4.8 mg and 6.2 mg (for 4%), between 4.85 mg and 6.15 mg (for 3%), between 4.9 mg and 6.1 mg (for 2%), or between 4.95 mg and 6.05 mg (for 1%). Furthermore, particularly with respect to biological systems or processes, the terms can mean up to an order of magnitude or up to 5-fold of a value. When particular values or compositions are provided in the application and claims, unless otherwise stated, the meaning of “about” or “consisting essentially of” should be assumed to be within an acceptable error range for that particular value or composition.

As used herein, “administration” or “administering” refers to the introduction of a composition into a subject by a chosen route. For example, if the chosen route is intravenous, the composition is administered by introducing the composition into a vein of the subject. In some examples, the peptides and antibodies disclosed herein are administered to a subject.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

As used herein, “polypeptide,” “oligopeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

As used herein, “polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In embodiments, the mammal is human.

As used herein, “antibody” refers to single chain, two-chain, and multi-chain proteins and glycoproteins belonging to the classes of polyclonal, monoclonal, chimeric and hetero immunoglobulins (monoclonal antibodies being preferred); it also includes synthetic and genetically engineered variants of these immunoglobulins.

As used herein, “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides/epitopes). In some embodiments, the affinity of one molecule for another molecule to which it specifically binds is characterized by a KD (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower KD. As used herein, the “binding” and “specific binding” of the first region to TAM receptor and the second region to a target substance do not require modulating, changing, affecting, or modifying activity of the bound TAM receptor or the target substance.

As used herein, “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

“Fv” is the minimum antibody fragment, which contains a complete antigen-recognition and -binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The “Fab fragment” also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

As used herein, the term “antibody fragment” or “antigen-binding fragment” or “active fragment” is defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules, (2) single chain polypeptides containing only one light chain variable domain, or a fragment thereof that contains the three CDRs of the light chain variable domain, without an associated heavy chain moiety, (3) single chain polypeptides containing only one heavy chain variable region, or a fragment thereof containing the three CDRs of the heavy chain variable region, without an associated light chain moiety, (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s), and (5) an isolated complementarity determining region (CDR).

The terms “phagocytic cells” and “phagocytes” are used interchangeably herein to refer to a cell that is capable of phagocytosis. There are four main categories of phagocytes: macrophages, mononuclear cells (histiocytes and monocytes), polymorphonuclear leukocytes (neutrophils), and dendritic cells.

As used herein “chimeric” refers to a molecule that includes sequences derived from two different molecules.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3, and when the Fc region is employed as a scaffold according to embodiments of the present disclosure, the Fc region may comprise CH2, CH3, or combinations thereof. The Fc region as a scaffold or in a heavy chain of an antibody may contain mutations. For example, heavy chain constant region or Fc region may contain substitutions selected from T250Q/M428L; M252Y/S254T/T256E+H433K/N434F; E233P/L234V/L235A/G236A+A327G/A330S/P331S; E333A; S239D/A330L/1332E; P2571/Q311; K326W/E333S; S239D/1332E/G236A; N297A; L234A/L235A; N297A+M252Y/S254T/T256E; K322A and K444A, wherein the numbering is according to the EU numbering (Edelman, G. M. et al., Proc. Natl. Acad. USA, 63, 78-85 (1969); www.imgt.org/IMGTScientificChart/Numbering/Hu_IGHGnber.html#refs).

As used herein, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof.

A “native sequence Fc region” or “wile-type Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, at least about 91%, at least about 92%, at least about 93%, at least about 94%, at least about 95%, at least about 96%, at least about 97%, at least about 98%, at least about 99% sequence identity therewith.

A polynucleotide or polypeptide having a certain percent “sequence identity” to another polynucleotide or polypeptide, means that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web (www) at ncbi.nlm.nih.gov/BLAST. See, e.g., Altschul et al. (1990), J. Mol. Biol. 215:403-10. Another alignment algorithm is FASTA, available in the Genetics Computing Group (GCG) package. See also at ebi.ac.uk/Tools/sss/fasta/, Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970), doi:10.1016/0022-2836(70)90057-4.

As used herein, an “effective dosage” or “effective amount” drug, compound, or pharmaceutical composition is an amount sufficient to effect beneficial or desired results. For prophylactic use, beneficial or desired results include results such as eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as inhibiting, suppressing or reducing the formation of substance accumulation (non-limiting example may include amyloid plaques), reducing, removing, clearing amyloid plaques, improving cognition, reversing or slowing cognitive decline, sequestering or increasing soluble substance circulating in biological fluids, decreasing one or more symptoms resulting from the disease (biochemical, histological and/or behavioral), including its complications and intermediate pathological phenotypes presenting during development of the disease, increasing the quality of life of those suffering from the disease, decreasing the dose of other medications required to treat the disease, enhancing effect of another medication, delaying the progression of the disease, and/or prolonging survival of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “treatment” or “treating” is an approach for obtaining beneficial or desired results including clinical results. For purposes of this invention, beneficial or desired clinical results include, but are not limited to, one or more of the following: inhibiting, suppressing or reducing the formation of deposit of substance, reducing, removing, or clearing amyloid deposits, improving cognition, reversing or slowing cognitive decline, sequestering soluble substance circulating in biological fluids, reducing a substance (including soluble, oligomeric and deposited) in a tissue, inhibiting, slowing and/or reducing accumulation of substance in the tissue, inhibiting, slowing and/or reducing toxic effects of a substance peptide in a tissue, decreasing symptoms resulting from the disease, increasing the quality of life of those suffering from the disease, decreasing the dose of other medications required to treat the disease, delaying the progression of the disease, and/or prolonging survival of patients. The tissue may include brain of an individual.

The term “development” of a disease means the onset and/or progression of the disease within an individual. A disease development can be detectable using standard clinical techniques as described herein. However, development also refers to disease progression that may be initially undetectable. For purposes of this invention, progression refers to the biological course of the disease state, in this case, as determined by a standard neurological examination, patient interview, or may be determined by more specialized testing. A variety of these diagnostic tests include, but not limited to, neuroimaging, detecting alterations of levels of specific proteins in the serum or cerebrospinal fluid (e.g., amyloid peptides and Tau), computerized tomography (CT), and magnetic resonance imaging (MRI). “Development” includes occurrence, recurrence, and onset. As used herein “onset” or “occurrence” of a disease includes initial onset and/or recurrence.

As used herein, “delaying” development of a disease means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. For example, a method that delays development of a disease is a method that reduces probability of disease development in a given time frame and/or reduces extent of the disease in a given time frame, when compared to not using the method. Such comparisons are typically based on clinical studies, using a statistically significant number of subjects.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well-known conventional methods.

### [TAM Receptor]

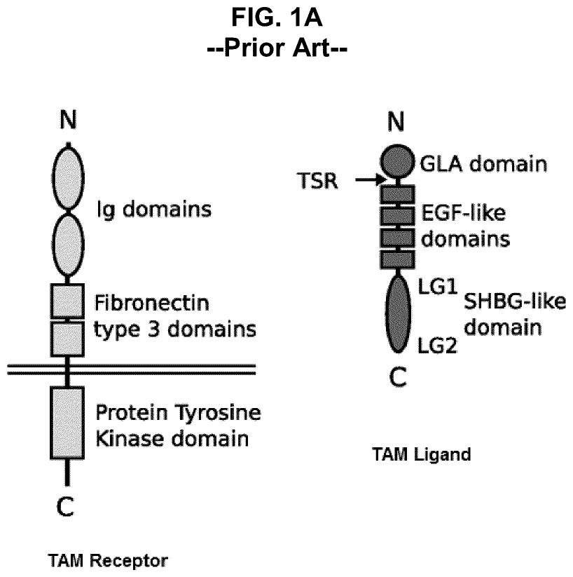

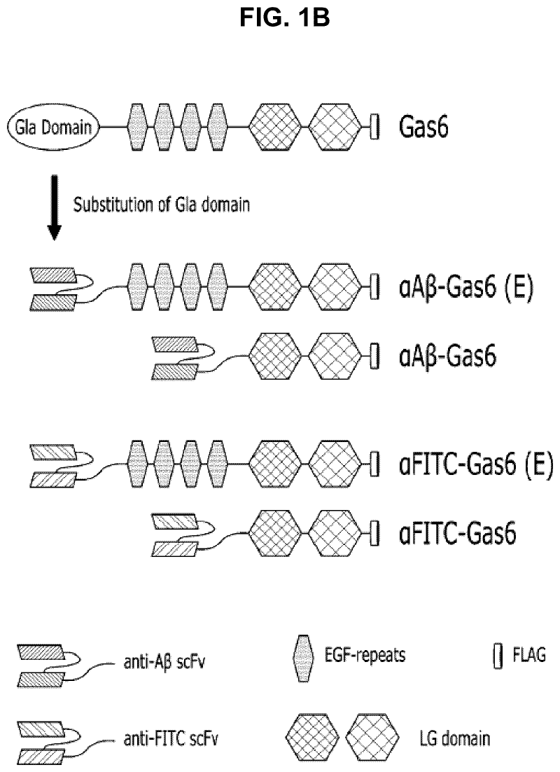

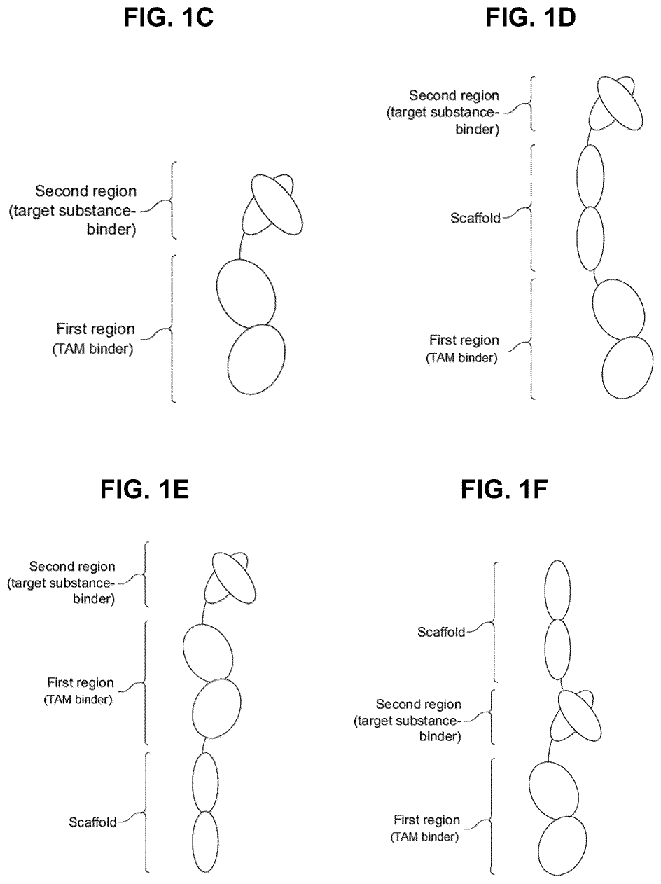

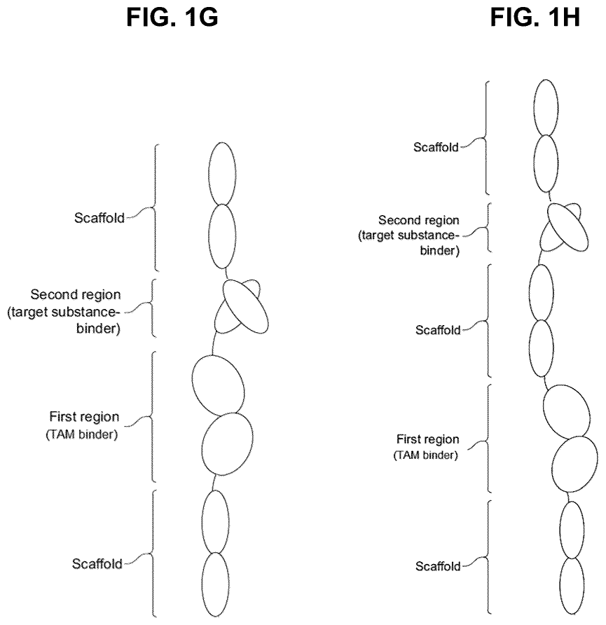

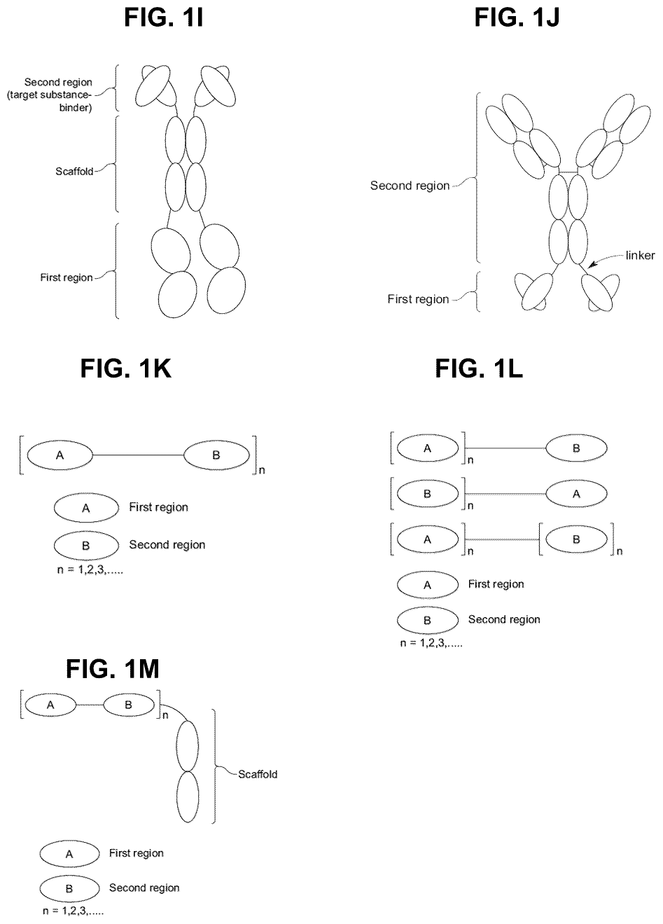

TAM receptors (Tyro3, Axl, and Mer) belong to a family of receptor tyrosine kinases that have important effects on homeostasis and inflammation. Also, they affect cell proliferation, survival, adhesion, and migration. TAM receptors comprise 2 immunoglobulin-like and 2 fibronectin type III repeats in their extracellular domains in tandem. This is connected to a single-pass transmembrane domain and a cytoplasmic protein tyrosine kinase. Left figure of FIG. 1A.

TAM receptors enhance phagocytosis of apoptotic cells, also known as efferocytosis.

The Axl protein contains 894 amino acids with a glycine-rich loop (Gly543-Gly548), a catalytic loop (His670-Asn677), and a DFG motif (Asp690-Phe691-Gly692). Although the molecular weight of the full-length Axl is 104 kDa, post-translational modifications of the extracellular domains give rise to two modified forms with molecular weights 120 and 140 kDa. Potential N-linked glycosylation sites include Asn43, Asn157, Asn198, Asn339, Asn345, and Asn401. In various embodiments of the present disclosure, the term “Axl” or “Axl receptor” or “Axl protein” includes the full-length Axl of 104 kDa, post-translational modified Axl, and glycosylated Axl. In some embodiments, the human Axl polypeptide corresponds to Genbank accession no. NP_068713 or NP_068713.2 (isoform 1 precursor), SEQ ID NO: 114, or UniProt accession no. P30530.4, Q8N5L2, or Q9UD27, or their mature forms. Amino acid residues 1-32 of SEQ ID NO: 114 is reported as signal sequence, and regions ranging amino acid residues 26 to 92 of SEQ ID NO: 114 is reported as interaction with Gas6. In one embodiment, the nucleic acid encoding the human Axl polypeptide corresponds to Genbank accession no. NM_021913, version no. NM_021913.5. Murine Axl refers to the Axl member of the murine TAM family of receptor tyrosine kinases. In some embodiments, the murine Axl polypeptide corresponds to Genbank accession no. AAH46618, version no. AAH46618.1, SEQ ID NO: 115. In one embodiment, the nucleic acid encoding the murine Axl polypeptide corresponds to Genbank accession no. BC046618, version no. BC046618.1. Various natural variants and mutations, and posttranslational variants and mutants of Axl as well as orthologues of Axl have been reported. For example, human Axl proteins under accession nos. NP_001265528.1 (626 amino acid residues), NP_001690.2 (885 amino acid residues, isoform 2 precursor), EAW57022 (885 amino acid residues, isoform CRA_a), EAW57023.1 (894 amino acid residues, isoform CRA_b), AAH32229.1 (894 amino acid residues), AAH32229.1 (885 amino acid residues), and the like are considered as “Axl” or “Axl receptor” or “Axl protein” according to the embodiments of the present disclosure.

The cells expressing the TAM receptor(s) may be at least one type of professional phagocytes, at least one type of non-professional phagocytes, or a combination thereof. Here, the professional phagocytes refer to cells whose main role is to remove dead cells and accumulated debris through phagocytosis, and examples thereof include macrophages, neutrophils, dendritic cells, and mast cells. Macrophages usually stay in each tissue that can become a path of infection, and in many cases, they are called different names for tissues, including, for example, adipose tissue macrophages, bone marrow or blood monocytes, hepatic Kupffer cells, lymph node sinus histiocytes, alveolar macrophages, connective tissue histiocytes or giant cells, microglia of the central nervous system, placental Hofbauer cells, renal intraglomerular mesangial cells, bone osteoclasts, epithelioid cells of granulomas, red pulp macrophages of the spleen, peritoneal macrophage of the peritoneal cavity, LysoMac of Peyer's patch, and the like. On the other hand, the non-professional phagocytes refer to cells that mainly perform functions specific to the tissue in which the phagocytes reside, but can perform phagocytosis when necessary, and examples thereof epithelial cells, endothelial cells, fibroblasts, mesenchymal cells, some tissue-specific cells, for example, astrocytes or oligodendrocyte of the central nervous system, retinal Muller glia, hepatocytes, muscular satellite cells, testicular Sertoli cells, etc., and some lymphocytes such as natural killer cells, large granular lymphocytes, eosinophils, basophils, B cells, etc. The fusion molecule according to the present disclosure is able to induce phagocytosis in phagocytes specific to a tissue in which a target substance to be cleared accumulates. For example, when abnormal proteins accumulated in the brain are to be cleared, the phagocytosis may be induced in astrocytes, microglia, oligodendrocytes, or combinations thereof. It may be induced, for example, by topically administering the fusion molecule according to the present disclosure to this tissue or by manipulating cells in the tissue to express and secrete the fusion molecule.

### [First Region Comprising a Sequence Capable of Binding to Tam Receptor]

TAM receptors can be activated via their ligands, growth arrest specific 6 protein (Gas6) and Protein S (Pros1), which are members of the family of vitamin K-dependent proteins.

In exemplary embodiments, the first region that is capable of binding to TAM receptors may comprise, consist of, or consist essentially of one or more TAM ligands.

A TAM ligand, protein S contains an amino terminal 7 carboxyglutamic acid (GLA) domain, followed by a thrombin-sensitive loop region and 4 epidermal growth factor-like domains ending with the carboxy-terminal (C-terminal), consisting of 2 laminin G repeats that together comprise the sex hormone-binding globulin domain (right figure of FIG. 1A). The C-terminal region is sufficient for TAM receptor binding and phosphorylation. Gas6 is a 75-kDa vitamin K-dependent protein and has high structural homology (˜42%) with protein S and the modular composition is the same as shown in FIG. 1A.

In addition to Gas6 (SEQ ID NO: 7) and ProS1 (SEQ ID NO: 34), tubby (accession nos. P50607, U54644.1, AAB53494.1, U82467.1, AAB53699.1, CH471064.2, EAW68634.1, BC075031.2, AAH75031.1, BC075032.2, AAH75032.1, NP_003311.2, NP_813977.1, 1531_A), tubby-like protein 1 (Tulp1) (accession nos. AAB53700.1, AAH32714.1, AAH65261.1, NP_001276324.1, AAB97966.1, EAX03840.1, EAX03839.1, BAJ84064.1, BAJ84063.1, AKU84911.1, NP_813977.1, NP_003311.2), and galectin-3 (Gal3) (accession nos. NP_002297, NP_002297.1) are reported as TAM receptor ligands. Tubby and Gal-3 specifically bind to Mer, whereas Tulp1 can activate all 3 of the TAM receptors.

Gas6, one of the ligands for TAM receptors, is reported to show the highest affinity for Axl compared to Tyro3 or Mer. Human Gas6 contains 678 amino acids (SEQ ID NO: 7), with gamma-carboxyglutamic acid (Gla) domains, four epidermal growth factor (EGF)-like domains, and two laminin G-like (LG) domains (FIG. 1A, right figure). Various isoforms of GAS6 are reported. For example, S6L, G8R, G8V, R14H, L18Q isoforms have been reported and these isoforms are included in the present disclosure.

In embodiments, the first region that is capable of binding to TAM receptor may comprise, consist of, or consist essentially of Gas6 protein or an active fragment thereof. The term “active fragment” as used herein denotes a fragment that is capable of binding to TAM receptor, in particular, Axl receptor. For example, an active fragment of Gas6 protein may comprise, consist of, or consist essentially of the sequence of SEQ ID NO: 1, 2, 5, 8, 9, 10, 11, 12, 13, 14, 15, 16, 17, 18, 19, 20, 21, 22, 23, 24, 25, 26, 27, 28, 29, 30, 31, 32, 33, 63, 64, 65, 66, 67, 68, 69, 70, 71, 72, 73, 74, 75, 76, 77, 78, 79, 80, 81, 82, 83, 84, 85, 86, or 87. For example, an active fragment of ProS protein may comprise, consist of, or consist essential of the sequence of SEQ ID NO: 3, 4, 6, 35, 36, 37, 38, 39, 40, 41, 42, 43, 44, 45, 46, 47, 48, 49, 50, 51, 52, 53, 54, 55, 56, 57, 58, 59, 60, 61, 62, 88, 89, 90, 91, 92, 93, 94, 95, 96, 97, 98, 99, 100, 101, 102, 103, 104, 105, 106, 107, 108, 109, 110, 111, 112, or 113. The present disclosure encompass the sequences having sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the sequence of any one of SEQ ID NOs. Sequences of SEQ ID NOS: 8-23 show sequence identity of at least 85% to SEQ ID NO: 1 (LG-1 domain of Gas6). Sequences of SEQ ID NOS: 24-33 show sequence identity of at least 85% to SEQ ID NO: 2 (LG-2 domain of Gas6). Sequences of SEQ ID NOS: 35-45 show sequence identity of at least 85% to SEQ ID NO: 3 (LG-1 domain of ProS). Sequences of SEQ ID NOS: 46-62 show sequence identity of at least 85% to SEQ ID NO: 4 (LG-2 domain of ProS). Sequences of SEQ ID NOS: 63-87 show sequence identity of at least 85% to SEQ ID NO: 5 (LG domains of Gas6). Sequences of SEQ ID NOS: 88-113 show sequence identity of at least 84% to SEQ ID NO: 6 (LG domains of ProS).

In other embodiments, the first region may comprise, consist, or consist essentially of variable region or CDRs of an anti-Axl antibody or a full-length anti-Axl antibody of which the effector function, in particular, Fc receptor-binding function is abolished or removed. The antibody or antigen-binding fragments may bind to extracellular domain of Axl, for example expressed on surface of phagocytic cells and induce internalization and phagocytosis without involving inflammatory reaction, in particular Fc-mediated inflammatory reaction. Non-limiting examples of anti-Axl antibody may include those described in, for example, WO2017200493A1, WO2015193430A1, WO2011159980A1, WO2016097370A1, WO2012175691A1, WO2015193428A1, WO2010131733A1, WO2017220695A1, WO2010130751A1, WO2016166302A1, WO2017009258A1, WO2016005593A1, and the like, all of which the contents are incorporated by reference herein in their entireties. According to embodiments of the present disclosure, whole antibody, variable region, CDRs, or scFv, F(ab), or F(ab′) of those anti-Axl antibodies may be employed as the first region of the fusion molecule. In certain embodiments, anti-Axl antibody may be anti-Axl agonistic antibodies or antigen-binding fragments thereof. The antibody or an antigen-binding fragment thereof may be selected from among, for example, i) immunoglobulins such as IgG1, IgG2, IgG3 and IgG4; ii) native antibody fragments such as Fv, Fab, Fab′, F(ab′)2, VHH, VNAR, etc.; and iii) engineered antibodies such as scFv, dsFv, ds-scFv, (scFv)2, diabody, triabody, tetrabody, pentabody, etc. The antibody or antigen-binding fragment thereof may be, for example, a Mab, Fab, or single-chain variable fragment (scFv) based on an antibody that specifically binds to a corresponding target substance, or six complementarity-determining regions (CDRs) derived from the antibody.

In other embodiments, the first region may comprise, consist, or consist essentially of variable region or CDRs of an anti-MerTK (Mer Tyrosine Kinase) antibody or a full-length anti-MerTK antibody of which the effector function, in particular, Fc receptor-binding function is abolished or removed. The antibody or antigen-binding fragments may bind to extracellular domain of MerTK, for example expressed on surface of phagocytic cells and induce internalization and phagocytosis without involving inflammatory reaction, in particular Fc-mediated inflammatory reaction. Non-limiting examples of anti-MerTK antibody may include those described in, for example, WO2016106221A1, WO2020076799A1, WO2020176497A1, and the like, all of which the contents are incorporated by reference herein in their entireties.

According to embodiments of the present disclosure, whole antibody, variable region, CDRs, or scFv, F(ab), or F(ab′) of those anti-MerTK antibodies may be employed as the first region of the fusion molecule. The antibody or an antigen-binding fragment thereof may be selected from among, for example, i) immunoglobulins such as IgG1, IgG2, IgG3 and IgG4; ii) native antibody fragments such as Fv, Fab, Fab′, F(ab′)2, VHH, VNAR, etc.; and iii) engineered antibodies such as scFv, dsFv, ds-scFv, (scFv)2, diabody, triabody, tetrabody, pentabody, etc. The antibody or antigen-binding fragment thereof may be, for example, a Mab, Fab, or single-chain variable fragment (scFv) based on an antibody that specifically binds to a corresponding target substance, or six complementarity-determining regions (CDRs) derived from the antibody.

In other embodiments, the first region may comprise, consist, or consist essentially of variable region or CDRs of an anti-Tyro3 antibody or a full-length anti-Tyro3 antibody of which the effector function, in particular, Fc receptor-binding function is abolished or removed. The antibody or antigen-binding fragments may bind to extracellular domain of Tyro3, for example expressed on surface of phagocytic cells and induce internalization and phagocytosis without involving inflammatory reaction, in particular Fc-mediated inflammatory reaction. Non-limiting examples of anti-Tyro3 antibody may include those described in, for example, WO2016166348A1, and the like, all of which the contents are incorporated by reference herein in their entireties. According to embodiments of the present disclosure, whole antibody, variable region, CDRs, or scFv, F(ab), or F(ab′) of those anti-Tyro3 antibodies may be employed as the first region of the fusion molecule. The antibody or an antigen-binding fragment thereof may be selected from among, for example, i) immunoglobulins such as IgG1, IgG2, IgG3 and IgG4; ii) native antibody fragments such as Fv, Fab, Fab′, F(ab′)2, VHH, VNAR, etc.; and iii) engineered antibodies such as scFv, dsFv, ds-scFv, (scFv)2, diabody, triabody, tetrabody, pentabody, etc. The antibody or antigen-binding fragment thereof may be, for example, a Mab, Fab, or single-chain variable fragment (scFv) based on an antibody that specifically binds to a corresponding target substance, or six complementarity-determining regions (CDRs) derived from the antibody.

The peptide comprising the sequence of any one of SEQ ID Nos above includes not only the amino acid sequence of the peptide but also an amino acid sequence variant thereof. The term “sequence variant” refers to a protein having a sequence in which one or more amino acid residues differ from the amino acid sequence. As long as the activity of the fusion molecule is maintained, any truncation, deletion, insertion, substitution, or a combination thereof in the final structure of the protein is possible. One example of the sequence variant is a form in which amino acid residues at sites not essential for activity are truncated or deleted, or amino acid residues at sites important for autoinhibition are substituted. In some cases, it may also be modified by phosphorylation, glycosylation, methylation, farnesylation, or the like. These sequence variations and modifications are more preferable when the function and/or stability (thermal stability, pH stability, structural stability, etc.) and/or solubility of the protein are increased by mutation in the amino acid sequence.

The method for mutagenesis of the amino acid sequence is based on a method of producing a nucleic acid molecule comprising a nucleotide sequence corresponding to the amino acid sequence to be mutated by mutating a nucleotide sequence encoding the protein, and a method for obtaining the gene encoding the protein may be performed in vivo or in vitro using any mutagenesis technique well known in the art, for example, site-directed mutagenesis (Hutchinson et al., J. Biol. Chem., 253:6551, 1978; Zoller and Smith, DNA, 3:479-488, 1984; Oliphant et al., Gene, 44:177, 1986; Hutchinson et al., Proc. Natl. Acad. Sci. U.S.A., 83:710, 1986), TAB linker (Pharmacia), PCR technique (Higuchi, 1989, “Using PCR to Engineer DNA” in PCR Technology: Principles and Applications for DNA Amplification, H. Erlich, ed., Stockton Press, Chapter 6, pp. 61-70), or the like.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the serum half-life of the antibody.

Examples of modified polypeptides include polypeptides with conservative substitutions of amino acid residues, one or more deletions or additions of amino acids which do not significantly deleteriously change the functional activity, or use of chemical analogs.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in Table 2 under the heading of “conservative substitutions”. If such substitutions result in a change in biological activity, then more substantial changes, denominated “exemplary substitutions” in Table 2, or as further described below in reference to amino acid classes, may be introduced and the products screened.

TABLE 2
Amino acid substitutions

| Original | Conservative | |
|---|---|---|
| Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
Non-polar: Norleucine, Met, Ala, Val, Leu, Ile;
Polar without charge: Cys, Ser, Thr, Asn, Gln;
Acidic (negatively charged): Asp, Glu;
Basic (positively charged): Lys, Arg;
Residues that influence chain orientation: Gly, Pro; and
Aromatic: Trp, Tyr, Phe, His.

Non-conservative substitutions are made by exchanging a member of one of these classes for another class. Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

Amino acid modifications can range from changing or modifying one or more amino acids to complete redesign of a region, such as the variable region. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. In still other embodiments, the CDR domain is CDR H3 and/or CDR L3.

### [Target Substances and Diseases Characterized by Aberrant Accumulation or Deposit of Target Substances]

The target substance may be a substance that accumulates in living tissue, causing a disease. For example, it may be a substance accumulated in an affected (i.e., diseased) tissue of a patient. The substance accumulated in a disease may be protein. That is, the disease may be proteopathy, without being limited thereto. For example, the target substance may be amyloid. That is, the proteopathy may be amyloidosis. The target substance may be selected from abnormally accumulated substances listed in Table 1 above, and in this case, the disease may be a disease in which each abnormally accumulated substance is detected. In some embodiments, the target substance, of which aberrant accumulation is associated with proteopathy and which is desired to be cleared or reduced or suppressed, may be APOE or apoptosis-associated spec-like protein containing a caspase activating recruitment domain (ASC-speck). For example, the proteopathy may be selected from Alzheimer's disease, Parkinson's disease, Huntington's disease, and Prion disease, and in this case, target substances may be abnormally accumulated proteins that cause the diseases. That is, the target substances may be β-amyloid, tau, α-synuclein, huntingtin, and prion proteins, respectively.

Aberrant accumulation of APOE is associated with Alzheimer's disease, cerebral amyloid angiopathy, and/or cardiovascular disease. Aberrant accumulation of apoptosis-associated spec-like protein containing a caspase activating recruitment domain (ASC-speck) is associated with Alzheimer's Disease, Parkinsons's Disease, Huntington's disease, Multiple System Atrophy, Amyotrophic Lateral Sclerosis, Sinocerebellar ataxia. Frontotemporal Dementia, Frontotemporal Lobar Degeneration, Mild Cognitive Impairment, Parkinson-plus syndromes, Pick disease, Progressive isolated aphasia, Grey-matter degeneration [Alpers], Subacute necrotizing encephalopathy, and Lewy body dementia.

### [Second Region of Fusion Molecule]

The second region that specifically binds to the target substance may be selected from among an antibody, an antigen-binding fragment thereof, an antibody-like protein, a peptide, an aptamer, and a soluble receptor, and is not particularly limited as long as it specifically binds to the target substance.

Here, the antibody or an antigen-binding fragment thereof may be selected from among, for example, i) immunoglobulins such as IgG1, IgG2, IgG3 and IgG4; ii) native antibody fragments such as Fv, Fab, Fab′, F(ab′)2, VHH, VNAR, etc.; and iii) engineered antibodies such as scFv, dsFv, ds-scFv, (scFv)2, diabody, triabody, tetrabody, pentabody, etc. The antibody or antigen-binding fragment thereof may be, for example, a Mab, Fab, or single-chain variable fragment (scFv) based on an antibody that specifically binds to a corresponding target substance, or six complementarity-determining regions (CDRs) derived from the antibody. That is, the protein or antigen-binding fragment thereof that specifically binds to the target substance comprises a portion necessary for an activity that specifically binds to the target substance, and the type or range thereof is not particularly limited as long as the protein or antigen-binding fragment thereof is linked to the first region and does not cause an inflammatory response and synaptic damage. For example, the target substance may be beta-amyloid, and in this case, the protein or antigen-binding fragment thereof that specifically binds to the target substance may comprise aducanumab or a single-chain variable fragment thereof. The second region comprise a Mab, Fab, or single-chain variable fragment based on based on six complementarity determining regions (CDRs) derived from commercially available antibodies such as aducanumab, semorinemab, and cinpanemab.

In non-limiting exemplary embodiments, the sequence information of the target substance can be obtained from public database and the target substance-binding sequences can be obtained from the publications or public database. By way of exemplary embodiments, some sequences from the public database are exemplified below. One skilled art should understand that sequences of target substance or a second region capable of binding to the target substance are not limited to the specific sequences exemplified below, but encompass isomers, orthologues, variants, and mutants. For example, when Amyloid precursor protein-derived β-amyloid is a target substance, the target subject may have the sequence available under GenBank accession no. AAB29908.1 or a fragment thereof (e.g., beta-amyloid (29-40) or a Chain A (Accession No. 1BJC_A)), and the second region that is capable of binding to the target substance can have a light chain variable region of SEQ ID NO: SEQ ID NO: 161 and a heavy chain variable region of SEQ ID NO: 162. When the target substance is a-Synuclein, it can have the sequence available under UniProtKB/Swiss-Prot: P37840.1 or a fragment thereof, and the second region capable of binding to the target substance may have light chain variable region of SEQ DI NO: 163 and a heavy chain variable region of SEQ ID NO: 164. When the target substance is Microtubule-associated protein tau, it can have the sequence available under UniProtKB/Swiss-Prot: P10636.5 or a fragment thereof, and the second region capable of binding to the target substance may have light chain variable region of SEQ DI NO: 165 and a heavy chain variable region of SEQ ID NO: 166. When the target substance is PrPsc, it can have the sequence available under GenBank Accession No. NP_001073592.1 or a fragment thereof, and the second region capable of binding to the target substance may be the sequences disclosed in US 2021/0070870 A1. When the target substance is Huntingtin exon 1, it can have the sequence available under GenBank Accession No. NP_001375421.1 or a fragment thereof, and the second region capable of binding to the target substance may be the sequences disclosed in US 2022/0332808 A1. When the target substance is TAR DNA-binding protein 43 (TDP43), it can have the sequence available under UniProtKB/Swiss-Prot: Q13148.1 or a fragment thereof, and the second region capable of binding to the target substance may be the sequences disclosed in U.S. Pat. No. 9,587,014 B2. When the target substance is superoxide dismutase 1 (SOD1), it can have the sequence available under GenBank: CAG46542.1 or a fragment thereof, and the second region capable of binding to the target substance may be the sequences disclosed in U.S. Pat. No. 9,283,271 B2. When the target substance is an immunoglobulin light-chain fragment, it can have the sequence available under PDB: 6Z1O_A or a fragment thereof, and the second region capable of binding to the target substance may be the sequences disclosed in U.S. Pat. No. 8,268,973 B2. When the target substance is an N-terminal fragment of serum amyloid A protein, it can have the sequence available under GenBank: AAB24060.1, GenBank: AAA85338.1, NCBI NP_001372595.1, or NCBI Reference Sequence: NP_110381.2 or a fragment thereof, and the second region capable of binding to the target substance may be the sequences disclosed in U.S. Pat. No. 8,268,973 B2. When the target substance is a transthyretin, it can have the sequence available under UniProtKB/Swiss-Prot: P02766.1 or a fragment thereof, and the second region capable of binding to the target substance may be the sequences disclosed in U.S. Pat. No. 11,267,877 B2. When the target substance is an amylin, IAPP (AIAPP), it can have the sequence available under UniProtKB/Swiss-Prot:P10997 or a fragment thereof, and the second region capable of binding to the target substance may be the sequences disclosed in U.S. Pat. No. 10,882,902. When the target substance is an APOE, it can have the sequence available under UniProtKB/Swiss-Prot:P02649.1 or a fragment thereof, and the second region capable of binding to the target substance may be the sequences disclosed in US 2022/0411485A. When the target substance is an Apoptosis-associated Spec-like protein containing a Caspase Activating Recruitment Domain (ASC-speck), it can have the sequence available under UniProtKB/Swiss-Prot:Q9ULZ3.2 or a fragment thereof (e.g., US 2021/0079075A), and the second region capable of binding to the target substance may be the sequences disclosed in US No. 10,961,3-6 B22021/0079075A.

The antibody or antigen-binding fragment thereof may not comprise an Fc region, and preferably may comprise an Fc region variant that does not bind to an Fc receptor (particularly an Fc receptor). This Fc region variant may serve to improve properties such as purification. Fc variants with a reduced affinity to the human FcγRIIIA and/or FcγRIIA and/or FcγRI compared to a IgG Fc region by way of amino acid substitution are disclosed for example, WO2012130831 and U.S. Pat. No. 8,753,628, of which entire content is incorporated by reference herein. Fc regions may be aglycosylated or deglycosylated.

The antibody-like protein refers to a protein scaffold capable of specifically binding to a target substance, like an antibody. Antibody-like proteins may be designed to have a size of about 2 to 20 kDa, which is smaller than antibodies (about 150 kDa on average), and thus target a binding site that antibodies cannot reach. It is known that antibody-like proteins are more stable at high temperatures than antibodies and are much easier to synthesize using non-mammalian cells such as viruses and yeast or synthesize chemically, compared to antibodies.

As used herein, the term “aptamer” refers to a single-stranded DNA (ssDNA) or RNA having high specificity and affinity for a specific substance. Aptamers have a very high affinity for specific substances, are stable, may be synthesized in a relatively simple way, may be modified in various ways to increase the binding affinity thereof, and can target cells, proteins, and even small organic substances. Thus, the aptamers are characterized by having very high specificity and stability compared to antibodies that have already been developed. In addition, the aptamer may be produced through a known SELEX (Systematic Evolution of Ligands by Exponential enrichment) method. As this aptamer, an aptamer that specifically binds to, for example, beta-amyloid, tau, or alpha-synuclein, may be produced through a known SELEX (Systematic Evolution of Ligands by Exponential enrichment) method and then linked to the first region, thereby producing the fusion molecule according to the present invention.

The aptamer of the present disclosure is not limited as long as it is able to specifically bind to beta-amyloid, tau, or alpha-synuclein, and bases that are used for the aptamer may be selected from among A, G, C, U, and deoxy forms thereof, unless otherwise specified.

In addition, the aptamer may be modified by linkage of at least one, selected from the group consisting of polyethylene glycol (PEG), inverted deoxythymidine (idT), locked nucleic acid (LNA), 2′-methoxy nucleoside, 2′-amino nucleoside, 2′F-nucleoside, amine linker, thiol linker, and cholesterol, at the 5′-end region, intermediate region, 3′-end region, or both ends thereof in order to increase the stability thereof. Inverted deoxythymidine (idT) is a molecule that is generally used to prevent nuclease degradation of an aptamer having weak nuclease resistance. In the case of a nucleic acid unit, the 3′-OH of the previous nucleotide is attached to the 5′-OH of the next nucleotide to form a chain, but in the case of idT, the 3′-OH of the previous nucleotide to attached the 3′-OH of the next unit so that 5′-OH, not 3′-OH, is exposed. Thus, idT is a molecule that has the effect of inhibiting degradation by 3′ exonuclease, a type of nuclease. In exemplary embodiments, aptamers against beta-amyloid include, but are not limited to, those reported in Yan Zheng, Advances in aptamers against Aβ and applications in Aβ detection and regulation for Alzheimer's disease, Theranostics, 2022; 12(5): 2095-2114, of which the content is incorporated by reference herein in its entirety.

The soluble receptor of the present disclosure comprises a domain having an activity capable of binding to a target substance, that is, an endogenous ligand, wherein the domain may be one derived from an endogenous membrane receptor or an intracellular receptor, or a derivative thereof. In this case, the soluble receptor comprised in the second region of the fusion molecule of the present disclosure may preferably be one in which regions having activities other than binding to a target substance have been removed from the endogenous receptor. Exemplary soluble receptors that bind to beta-amyloid are reported by John E. Donahue et al., RAGE, LRP-1, and amyloid-beta protein in Alzheimer's disease, Acta Neuropathol (2006) 112:405-415, of which the content is incorporated by reference herein in its entirety.

In the embodiments, the peptide as a second region, means an entity other than the antibody or an active fragment thereof, antibody-like protein or soluble receptor among polypeptides having amino acids as monomers capable of binding specifically to a target substance. Various peptides that bind to beta amyloid are reported in, for example, Alexander L. Schwarzman, Selection of peptides binding to the amyloid b-protein reveals potential inhibitors of amyloid formation, Amyloid, December 2005; 12(4): 199-209, of which the content is incorporated by reference herein in its entirety.

Since the fusion molecule according to the present disclosure induces phagocytosis through interaction with the TAM receptor(s), the phagocytosis may be induced in cells expressing the TAM receptor(s). Phagocytosis generally means ingestion of cells or particles of 0.5 μm or more in size, and includes a process of tethering, engulfing, and degrading the cells or particles. In this case, phagocytosis forms a phagosome that surrounds the internalized cell or particle, and includes degradation within the phagolysosome by fusion of the phagosome and the lysosome. In phagocytosis, the process of cell death by apoptosis or necrosis is also referred to as efferocytosis.

### [Fusion Molecule or Binding Molecule]

The induction of phagocytosis by the fusion molecule according to the present disclosure may not involve an inflammatory response. This enables clearance of the target substance without inducing an inflammatory response and tissue damage caused by an inflammatory response to be suppressed so that tissue dysfunction caused by accumulation of the target substance can be treated more safely than conventional techniques.

According to the embodiments, the fusion molecule or the binding molecule does not contain the target substance or a fragment thereof, to which the first region binds.

The first region and the second regions, described above, are coupled to each other directly or via a linker, to form a fusion molecule. Embodiments of the fusion molecule according to the present disclosure encompass monomers of a polypeptide comprising the first region and the second region in a single chain as well as multimers composed of two or more polypeptide chains. Multimers encompass various forms of multimers such as homo-multimers and hetero-multimers. Therefore, according to the embodiments, the fusion molecule may have a monovalent first region (i.e., having one binding site to a single TAM receptor) or a multivalent first region(s) (i.e., having multiple first regions or having one single first region capable of binding to two or more TAM receptors). Embodiments of the fusion molecule according to the present disclosure may have a monovalent second region (i.e., having one binding site to a single target substance) or a multivalent second region(s) (i.e., having multiple second regions each binding different target substances or having one single second region capable of binding to two or more target substances). Embodiments of the fusion molecule according to the present disclosure may a monovalent first region and a monovalent second region; a monovalent first region and a multivalent second region; or a multivalent first region and a multivalent second region.

Schematic depiction of non-limiting exemplary embodiments of the fusion molecules are shown in FIGS. 1C through 1M.

FIG. 1C depicts fusion molecules comprising a first region and a second region coupled to each other. The first region of FIG. 1C may be any one of the first region described above, which is capable of binding to a TAM receptor. The first region may be a single TAM ligand or an antibody or antigen-binding fragment thereof. The first region may be of a plurality of same or different TAM binders. Similarly, the second region of FIG. 1C may be any one of the second region described above, which is capable of binding to a target substance. The second region may be a single target substance binder. The second region may be of a plurality of same or different target substance binders. A fusion molecule may comprise a scFv, Fab, nanobody, or antibody as a first region and a scFv, Fab, nanobody, or antibody as a second region, a TAM ligand or a fragment thereof as a first region and a scFv, Fab, nanobody, or antibody as a second region, a scFv, Fab, nanobody, or antibody as a first region and a ligand or receptor or an aptamer as a second region, and the like. For example, a scFv, Fab, or nanobody as a first region or a second region may be a part of an antibody as a second region or a first region, forming a bispecific or multispecific antibody as a fusion molecule. A single or tandem of a scFv, Fab, or nanobody as a first or a second region may be linked to a whole antibody as a second or a first region. When the fusion molecule is a form of bispecific antibody, the first region is not an anti-MerTK antibody or a fragment thereof. The fusion molecule may be of a monomeric structure, a tandem structure where the first region, the second region, or the entire fusion molecule are repeated, or a multimeric structure containing two or more peptide chains. In case of multimeric fusion molecules, individual peptide chain may have a same sequence (homomultimer) or different sequences (heteromultimer), For heteromultimeric fusion molecules, the first region and the second region may present in all of the plural peptide chains forming the multimeric structure, or in different peptide chains, respectively, or a part of the plural peptide chains has both the first and the second region and the other(s) of the plural peptide chains has only either of the first region or the second region. While not shown in FIG. 1C, the fusion molecule may comprise one or more linker to couple the first region and the second region.

FIGS. 1D-1M depict fusion molecules comprising a first region and a second region as well as a scaffold. The first region, second region, and scaffold are as those described in this disclosure. As depicted in FIGS. 1D-1M, the scaffold may be bond to the first region and/or second region at different positions. The first region of FIGS. 1D-1M may be any one of the first region described above, which is capable of binding to a TAM receptor. The first region may be a single TAM ligand or an antibody or antigen-binding fragment thereof. The first region may be of a plurality of same or different TAM binders. Similarly, the second region of FIGS. 1C-1M may be any one of the second region described above, which is capable of binding to a target substance. The second region may be a single target substance binder. The second region may be of a plurality of same or different target substance binders. A fusion molecule may comprise a scFv, Fab, nanobody, or antibody as a first region and a scFv, Fab, nanobody, or antibody as a second region, a TAM ligand or a fragment thereof as a first region and a scFv, Fab, nanobody, or antibody as a second region, a scFv, Fab, nanobody, or antibody as a first region and a ligand or receptor or an aptamer as a second region, and the like. For example, a scFv, Fab, or nanobody as a first region or a second region may be a part of an antibody as a second region or a first region, forming a bispecific or multispecific antibody as a fusion molecule. A single or tandem of a scFv, Fab, or nanobody as a first or a second region may be linked to a whole antibody as a second or a first region. When the fusion molecule is a form of bispecific antibody, the first region is not an anti-MerTK antibody or a fragment thereof. The fusion molecule may be of a monomeric structure, a tandem structure, or a multimeric structure containing two or more peptide chains. In case of multimeric fusion molecules, individual peptide chains may have a same sequence (homomultimer) or different sequences (heteromultimer). In case of heteromultimeric fusion molecules, the first region and the second region may present in all of the plural peptide chains forming the multimeric structure, or in different peptide chains, respectively, or a part of the plural peptide chains has both the first and the second region and the other(s) of the plural peptide chains has only either of the first region or the second region. While not shown in FIGS. 1D-1M, the fusion molecule may comprise one or more linker to couple the first region, the second region, and/or the scaffold. The fusion molecule may further comprise a tag. When such a label is added to the fusion molecule, it may be used to check the purification, expression, action or mechanism of action of the fusion molecule.

Examples of the tag include, but are not limited to, His-tag, T7-tag, S-tag, FLAG-tag, Strep-tag, thioredoxin (Trx)-tag, His-patch thioredoxin-tag, lacZ (L-galactosidase)-tag, chloramphenicol acetyltransferase-tag, trpE-tag, avidin/streptavidin/Strep-tag, T7gene10-tag, staphylococcal protein A-tag, streptococcal protein G-tag, glutathione-S-transferase (GST)-tag, dihydrofolate reductase (DHFR)-tag, cellulose binding domains (CBDs)-tag, maltose binding protein (MBP)-tag, galactose-binding protein-tag, calmodulin binding protein (CBP)-tag, hemagglutinin influenza virus (HAI)-tag, HSV-tag, B-(VP7 protein region of bluetongue virus)-tag, polycysteine-tag, polyphenyalanine-tag, (Ala-Trp-Trp-Pro).-tag, polyaspartic acid-tag, c-myc-tag, lac repressor-tag, and the like. The tag may be located at the N-terminus, C-terminus or internally of the target protein.

The fusion molecule may further comprise a signal peptide or leader sequence at the N-terminus. It is known that a signal peptide is a short peptide present at the N-terminus at the initial stage of protein synthesis toward the secretory pathway, and directs the intracellular localization of the corresponding protein, membrane topology (in the case of a membrane protein), and the like. The signal peptide may be cleaved during expression and extracellular secretion of the fusion molecule.

The above-mentioned first region, second region, tag, signal peptide, or regions having minimal functionality (e.g., LG1 and LG2 regions or scFv heavy chain variable region and light chain variable region) included in the fusion molecule may be linked together directly or by a linker comprising a short oligopeptide or polypeptide. In general, the linker may comprise 2 to 500 amino acid residues. The length or type of the linker is not particularly limited as long as the linker can link the above-described regions together so as to have the intended activity, thereby forming the fusion molecule. An example of the linker may be the commonly used oligopeptide linker (GGGGS)n (SEQ ID NO: 116), that is, a linker in which one or more Gly-Gly-Gly-Gly-Ser (SEQ ID NO: 117) units are repeated. Other examples of the linker include, but are not limited to, (GSSGGS)n (SEQ ID NO: 118), KESGSVSSEQLAQFRSLD (SEQ ID NO: 119), EGKSSGSGSESKST (SEQ ID NO: 120), GSAGSAAGSGEF (SEQ ID NO: 121), (EAAAK)n (SEQ ID NO: 122), CRRRRRREAEAC (SEQ ID NO: 123), A(EAAAK)₄ALEA(EAAAK)₄A (SEQ ID NO: 124), GGGGGGGG (SEQ ID NO: 125), GGGGGG (SEQ ID NO: 126), AEAAAKEAAAAKA (SEQ ID NO: 127), PAPAP (SEQ ID NO: 128), (Ala-Pro)n, VSQTSKLTRAETVFPDV (SEQ ID NO: 129), PLGLWA (SEQ ID NO: 130), TRHRQPRGWE (SEQ ID NO: 131), AGNRVRRSVG (SEQ ID NO: 132), RRRRRRRR (SEQ ID NO: 133), GFLG (SEQ ID NO: 134), and GSSGGSGSSGGSGGGDEADGSRGSQKAGVDE (SEQ ID NO: 135). Other suitable linkers comprise the sequences described in WO2012/088461A, of which the content is incorporated by reference herein in its entirety.

The fusion molecule according to embodiments of the present disclosure may further comprise a scaffold bound to the first region, to the second, or to both of the first region and the second region at different positions of the scaffold, as schematically depicted in FIGS. 1D-1D-1M. The scaffold refers to a protein or peptide, when incorporated into a protein or peptide of interest, improves properties of the total protein such as PK, enhances stability and/or in vivo half-life, or enhances productivity of the protein. In some embodiments, blood-brain barrier (BBB) permeable scaffold may optimize BBB permeability and/or optimizing distribution of the protein of interest (e.g., the fusion molecules according to the present disclosure) in the brain.

The scaffold may include, but not limited thereto, a single chain Fc region with reduced or abolished Fc receptor binding affinity, a multimer Fc region with reduced or abolished Fc receptor binding affinity, an antibody without variable region, or an Fc-hinge region with reduced or abolished Fc receptor binding affinity. In some embodiments, the scaffold may include albumin. The first region may be linked or fused to one position of the scaffold and the second region may be linked or fused to another position of the scaffold. The link or fusion between the first/the second region and the scaffold may be a direct bond or via a linker described above. It is known that heavy chain constant region or Fc region may contain mutations selected among T250Q/M428L; M252Y/S254T/T256E+H433K/N434F; E233P/L234V/L235A/G236A+A327G/A330S/P331S; E333A; S239D/A330L/1332E; P2571/Q311; K326W/E333S; S239D/1332E/G236A; N297A; L234A/L235A; N297A+M252Y/S254T/T256E; K322A and K444A, wherein the numbering is according to the EU numbering (Edelman, G. M. et al., Proc. Natl. Acad. USA, 63, 78-85 (1969); imgt.org/IMGTScientificChart/Numbering/Hu_IGHGnber.html#refs).

The fusion molecules according to aspects of the present disclosure may have a structure as schematically shown in non-limiting exemplary illustrations of, for example, FIG. 1B and FIGS. 1C through 1D-1M.

Structures of non-limiting exemplary fusion molecules (including signal sequences, optional linkers, and tags) are illustrated in Tables 3 and 5-13 and SEQ ID NOs: 136, 138, 140, 142, 150, 152, 154, 156, 158, 162-169. Non-limiting exemplary fusion molecules may comprise, consist of, or consist essentially of the fusion molecule (or binding molecule) of the sequence of amino acid residues 31-871 of SEQ ID NO: 136, amino acid residues 31-687 of SEQ ID NO:138, amino acid residues 31-697 of SEQ ID NO:140, amino acid residues 31-684 of SEQ ID NO: 150, amino acid residues 31-676 of SEQ ID NO: 152, amino acid residues 25-673 of SEQ ID NO: 154, amino acid residues 22-662 of SEQ ID NO: 156, or amino acid residues 22-885 of SEQ ID NO: 158, or a sequence having at least 90% sequence identity thereto, wherein a different linker can be used in place the linkers as shown in Tables 3 and 5-10. Another aspect of the present disclosure provides a nucleic acid molecule encoding the fusion molecule, and an expression vector containing the same.

As described above, the nucleic acid molecule sequence encoding the fusion molecule may be mutated by substitution, deletion, insertion, or a combination thereof, of one or more nucleotide residues, as long as it encodes a protein having an activity equivalent thereto.

The nucleic acid molecule sequence encoding the fusion molecule may be isolated from nature or artificially produced through synthesis or genetic recombination. The nucleic acid molecule sequence encoding the fusion molecule is operatively linked to an expression vector capable of expressing the same.

The term “expression vector” is a vector capable of expressing a protein or RNA of interest by introducing a nucleic acid sequence encoding a gene of interest into a suitable host cell, and refers to a gene construct containing essential regulatory elements operably linked to express the gene insert. Such expression vectors include all vectors such as plasmid vectors, cosmid vectors, bacteriophage vectors, and viral vectors.

A suitable expression vector has expression control elements such as a promoter, a start codon, a stop codon, a polyadenylation signal and an enhancer. The start codon and the stop codon are generally considered to be part of a nucleic acid sequence encoding a protein, and the sequence encoding the protein is designed to be in frame so as to be operable in the vector. The promoter may be constitutive or inducible. In addition, a conventional expression vector contains a selectable marker. Operational linkage with the expression vector can be performed using genetic recombination techniques well known in the art, and site-specific DNA cleavage and ligation can be performed using enzymes generally known in the art.

The expression vector may preferably be configured to express the fusion molecule in a host cell for isolation and purification of the fusion molecule or such that the vector may be introduced into a cell in vivo and the corresponding cell may express and secrete the fusion molecule. For the purpose of introducing into cells in vivo, the vector may preferably be a non-integrating vector, that is, a vector that is not integrated into the genome of a host cell.

Still another aspect of the present disclosure provides a cell expressing the fusion molecule.

The cells may be transformed to contain the nucleic acid molecule or an expression vector containing the same, and the “transformation” may be performed using suitable standard techniques selected depending on the host cell as known in the art, including any method of introducing the nucleic acid molecule into an organism, cell, tissue, or organ. These methods include, but are not limited to, electroporation, protoplast fusion, calcium phosphate (CaPO₄) precipitation, calcium chloride (CaCl₂) precipitation, agitation using silicon carbide fibers, Agrobacterium-mediated transformation, PEG-, dextran sulfate-, lipofectamine-, and desiccation/inhibition-mediated transformation methods.

Examples of the host cells include, but are not limited to, prokaryotic host cells such as Escherichia coli, Bacillus subtilis, Streptomyces, Pseudomonas (e.g., Pseudomonas putida), Proteus mirabilis, or Staphylococcus (e.g., Staphylococcus carnosus). Other examples of the host cell include fungal cells such as Aspergillus, yeast cells, including Pichia pastoris, Saccharomyces cerevisiae, Schizosaccharomyces, and Neurospora crassa, lower eukaryotic cells, or cells derived from higher eukaryotes including insect cells, plant cells, or mammalian cells.

After the fusion molecule is expressed in the cells, it may be isolated and purified using conventional biochemical isolation techniques, such as treatment with a protein precipitating agent (salting out method), centrifugation, sonication, ultrafiltration, dialysis, or various chromatography such as molecular sieve chromatography (gel filtration), adsorption chromatography, ion exchange chromatography, and affinity chromatography, which are generally used in combination in order to isolate proteins with high purity (Sambrook et al., Molecular Cloning: A laboratory Manual, 2nd Ed., Cold Spring Harbor Laboratory Press(1989); Deuscher, M., Guide to Protein Purification Methods Enzymology, Vol. 182. Academic Press. Inc., San Diego, CA (1990)).

### [Pharmaceutical Composition]

Yet another aspect of the present disclosure provides a pharmaceutical composition for preventing or treating a disease caused by accumulation of the target substance in living tissue, the pharmaceutical composition containing the fusion molecule or the expression vector. Here, the composition may be administered topically to a site where the substance that causes the disease, that is, the target substance, accumulates.

A further aspect of the present disclosure provides the use of the fusion molecule for manufacture of a medicament for preventing or treating proteopathy.

The fusion molecule, which is an active ingredient in the pharmaceutical composition, is contained in a “pharmaceutically effective amount”.

The pharmaceutical composition may be administered orally or parenterally, preferably parenterally. More preferably, it may be administered topically to a tissue in which the target substance to be cleared accumulates.

As used herein, the term “parenteral administration” includes subcutaneous injection, intravenous, intramuscular, intrasternal injection or infusion techniques.

When the pharmaceutical composition is prepared as an injectable formulation, it may be prepared as the injectable formulation a conventional method known in the art. The injectable formulation may be in a form dispersed in a sterile medium so that it may be administered directly to a patient or may be in a form that may be administered after being dispersed in distilled water for injection at an appropriate concentration.

When the pharmaceutical composition is formulated for oral administration, it may contain one or more carriers selected from among diluents, lubricants, binders, disintegrants, sweeteners, stabilizers, and preservatives, and may contain one or more additives selected from among flavorings, vitamins, and antioxidants.

Techniques necessary for formulation of the pharmaceutical composition, and pharmaceutically acceptable carriers, additives, etc. are widely known to those skilled in the art (see, for example, the Handbook of Pharmaceutical Excipients, 4^{th} edition, Rowe et al., Eds., American Pharmaceuticals Association (2003); Remington: the Science and Practice of Pharmacy, 20^{th} edition, Gennaro, Ed., Lippincott Williams & Wilkins (2000); Remington's Pharmaceutical Sciences (19^{th} ed., 1995)).

The appropriate dosage of the pharmaceutical composition may vary depending on factors such as formulation method, administration mode, patient's age, weight, sex, medical condition, diet, administration time, administration route, excretion rate, and response sensitivity. The dosage of the pharmaceutical composition of the present disclosure is 0.0001 to 1,000 g/kg body weight for an adult.

### Advantageous Effects

The present disclosure relates to a fusion molecule having phagocytosis-inducing activity, which can solve the problem of tissue damage caused by activation of an inflammatory response, which occurs in the prior art. Accordingly, the fusion molecule is able to effectively clear abnormally accumulated substances such as beta-amyloid, tau, alpha-synuclein, huntingtin or prion protein, and thus may be used to prevent or treat diseases caused by these abnormally accumulated substances, for example, Alzheimer's disease, Parkinson's disease, Huntington's disease, or prion disease. The fusion molecule may be administered to a patient in the form of a purified fusion molecule or a gene therapy vector capable of expressing and secreting the fusion molecule when introduced into a cell.

However, it should be understood that effects of the present disclosure are not limited to the above effects, and include all effects that may be inferred from the configuration of the invention described in the detailed description or claims.

### EXAMPLES

Hereinafter, the present disclosure will be described in more detail with reference to examples and experimental examples. However, the following examples and experimental examples are illustrative only, and the scope of the invention is not limited thereto.

### Preparation Example 1. Preparation of Gas6-Based Fusion Molecule Having Beta-Amyloid Clearance Activity (I): Beta-Amyloid Binding Region in the Form of scFv

To prepare a beta-amyloid (Aβ)-specific chimeric phagocytosis inducer based on Gas6 protein, the Gla domain, which recognizes PS (phosphatidylserine) in apoptotic cells, was first removed, and a single-chain variable fragment (scFv) of aducanumab, an amyloid-specific antibody, was introduced at that position [αAβ-Gas6(E)].

In addition, for the efficiency of protein production, the EGF repeat domain present in the internal residues of the Gas6 protein was also removed and an scFv of aducanumab was introduced at that position, thereby preparing αAβ-Gas6 (FIG. 1B).

In addition, as controls for verifying beta-amyloid-specific binding of the scFv of aducanumab, αFITC-Gas6(E) and αFITC-Gas6, each introduced with an E2 scFv that selectively recognizes FITC, instead of the scFv of aducanumab, were prepared.

Table 3 below shows amino acid sequences related to the preparation of the fusion molecules, and Table 4 below shows nucleotide sequences related to the preparation of the fusion molecules (the underlined sequences are flag tags). In Table 3, information for sequences constituting the final binding molecules are included within parenthesis. The full length sequences contain, from the N-terminal to the C-terminal direction, as an example, signal sequence (SS), first region, linker (when applicable), second region, and flag or his tags, which are linked consecutively. The sequence identifiers are intended for the full length sequences.

TABLE 3
1. αAβ-Gas6(E) (FLAG tag, Gla delete, G-/-) SEQ ID NO: 136
MAPSLSPGPAALRRAPQLLLLLLAAECALA (SS)
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPLTFGGGTKVEIKRGGGGSGGG
GSGGGGSEVQLVESGGGVVQPGRSLRLSCAASGFAFSSYGMHWVRQAPGKGLEWV
AVIWFDGTKKYYTDSVKGRFTISRDNSKNTLYLQMNTLRAEDTAVYYCARDRGIGA
RRGPYYMDVWGKGTTVTVSS (Adu-scFv)
GGGGSGGGGS (Linker)
CINKYGSPYTKNSGFATCVQNLPDQCTPNPCDRKGTQACQDLMGNFFCLCKAGWG
GRLCDKDVNECSQENGGCLQICHNKPGSFHCSCHSGFELSSDGRTCQDIDECADSEA
CGEARCKNLPGSYSCLCDEGFAYSSQEKACRDVDECLQGRCEQVCVNSPGSYTCHC
DGRGGLKLSQDMDTCEDILPCVPFSVAKSVKSLYLGRMFSGTPVIRLRFKRLQPTRL
VAEFDFRTFDPEGILLFAGGHQDSTWIVLALRAGRLELQLRYNGVGRVTSSGPVINH
GMWQTISVEELARNLVIKVNRDAVMKIAVAGDLFQPERGLYHLNLTVGGIPFHEKD
LVQPINPRLDGCMRSWNWLNGEDTTIQETVKVNTRMQCFSVTERGSFYPGSGFAFY
SLDYMRTPLDVGTESTWEVEVVAHIRPAADTGVLFALWAPDLRAVPLSVALVDYH
STKKLKKQLVVLAVEHTALALMEIKVCDGQEHVVTVSLRDGEATLEVDGTRGQSE
VSAAQLQERLAVLERHLRSPVLTFAGGLPDVPVTSAPVTAFYRGCMTLEVNRRLLD
LDEAAYKHSDITAHSCPPVEPAAA(Gas6-Gla deleted)
QGSRADYKDHDGDYKDHDIDYKDDDDK* (FLAG)
2. αFITC-Gas6(E) (FLAG tag, Gla delete, G-/-) SEQ ID NO: 137
MAPSLSPGPAALRRAPQLLLLLLAAECALA(SS)
QVQLVESGGNLVQPGGSLRLSCAASGFTFGSFSMSWVRQAPGGGLEWVAGLSARSS
LTHYADSVKGRFTISRDNAKNSVYLQMNSLRVEDTAVYYCARRSYDSSGYWGHFY
SYMDVWGQGTLVTVSGGGGSGGGGSGGGGSSVLTQPSSVSAAPGQKVTISCSGSTS
NIGNNYVSWYQQHPGKAPKLMIYDVSKRPSGVPDRFSGSKSGNSASLDISGLQSEDE
ADYYCAAWDDSLSEFLFGTGTKLTVLG (aFITC-scFv)
GGGGSGGGGS (Linker)
CINKYGSPYTKNSGFATCVQNLPDQCTPNPCDRKGTQACQDLMGNFFCLCKAGWG
GRLCDKDVNECSQENGGCLQICHNKPGSFHCSCHSGFELSSDGRTCQDIDECADSEA
CGEARCKNLPGSYSCLCDEGFAYSSQEKACRDVDECLQGRCEQVCVNSPGSYTCHC
DGRGGLKLSQDMDTCEDILPCVPFSVAKSVKSLYLGRMFSGTPVIRLRFKRLQPTRL
VAEFDFRTFDPEGILLFAGGHQDSTWIVLALRAGRLELQLRYNGVGRVTSSGPVINH
GMWQTISVEELARNLVIKVNRDAVMKIAVAGDLFQPERGLYHLNLTVGGIPFHEKD
LVQPINPRLDGCMRSWNWLNGEDTTIQETVKVNTRMQCFSVTERGSFYPGSGFAFY
SLDYMRTPLDVGTESTWEVEVVAHIRPAADTGVLFALWAPDLRAVPLSVALVDYH
STKKLKKQLVVLAVEHTALALMEIKVCDGQEHVVTVSLRDGEATLEVDGTRGQSE
VSAAQLQERLAVLERHLRSPVLTFAGGLPDVPVTSAPVTAFYRGCMTLEVNRRLLD
LDEAAYKHSDITAHSCPPVEPAAA(Gas6-Gla deleted)
QGSRADYKDHDGDYKDHDIDYKDDDDK* (FLAG)
3. αAβ-Gas6 (FLAG tag, Gla EGF delete, GE-/-) SEQ ID NO: 138
MAPSLSPGPAALRRAPQLLLLLLAAECALA(SS)
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPLTFGGGTKVEIKRGGGGSGGG
GSGGGGSEVQLVESGGGVVQPGRSLRLSCAASGFAFSSYGMHWVRQAPGKGLEWV
AVIWFDGTKKYYTDSVKGRFTISRDNSKNTLYLQMNTLRAEDTAVYYCARDRGIGA
RRGPYYMDVWGKGTTVTVSS (Adu-scFv)
GGGGSGGGGS (Linker)
DILPCVPFSVAKSVKSLYLGRMFSGTPVIRLRFKRLQPTRLVAEFDFRTFDPEGILLFA
GGHQDSTWIVLALRAGRLELQLRYNGVGRVTSSGPVINHGMWQTISVEELARNLVI
KVNRDAVMKIAVAGDLFQPERGLYHLNLTVGGIPFHEKDLVQPINPRLDGCMRSWN
WLNGEDTTIQETVKVNTRMQCFSVTERGSFYPGSGFAFYSLDYMRTPLDVGTESTW
EVEVVAHIRPAADTGVLFALWAPDLRAVPLSVALVDYHSTKKLKKQLVVLAVEHT
ALALMEIKVCDGQEHVVTVSLRDGEATLEVDGTRGQSEVSAAQLQERLAVLERHLR
SPVLTFAGGLPDVPVTSAPVTAFYRGCMTLEVNRRLLDLDEAAYKHSDITAHSCPPV
EPAAA (Gas6-Gla EGF deleted)
QGSRADYKDHDGDYKDHDIDYKDDDDK* (FLAG)
4. αFITC-Gas6 (FLAG tag, Gla EGF delete, GE-/-) SEQ ID NO: 139
MAPSLSPGPAALRRAPQLLLLLLAAECALA(SS)
QVQLVESGGNLVQPGGSLRLSCAASGFTFGSFSMSWVRQAPGGGLEWVAGLSARSS
LTHYADSVKGRFTISRDNAKNSVYLQMNSLRVEDTAVYYCARRSYDSSGYWGHFY
SYMDVWGQGTLVTVSGGGGSGGGGSGGGGSSVLTQPSSVSAAPGQKVTISCSGSTS
NIGNNYVSWYQQHPGKAPKLMIYDVSKRPSGVPDRFSGSKSGNSASLDISGLQSEDE
ADYYCAAWDDSLSEFLFGTGTKLTVLG (aFITC-scFv)
GGGGSGGGGS (Linker)
DILPCVPFSVAKSVKSLYLGRMFSGTPVIRLRFKRLQPTRLVAEFDFRTFDPEGILLFA
GGHQDSTWIVLALRAGRLELQLRYNGVGRVTSSGPVINHGMWQTISVEELARNLVI
KVNRDAVMKIAVAGDLFQPERGLYHLNLTVGGIPFHEKDLVQPINPRLDGCMRSWN
WLNGEDTTIQETVKVNTRMQCFSVTERGSFYPGSGFAFYSLDYMRTPLDVGTESTW
EVEVVAHIRPAADTGVLFALWAPDLRAVPLSVALVDYHSTKKLKKQLVVLAVEHT
ALALMEIKVCDGQEHVVTVSLRDGEATLEVDGTRGQSEVSAAQLQERLAVLERHLR
SPVLTFAGGLPDVPVTSAPVTAFYRGCMTLEVNRRLLDLDEAAYKHSDITAHSCPPV
EPAAA (Gas6-Gla EGF deleted)
QGSRADYKDHDGDYKDHDIDYKDDDDK* (FLAG)
5. αAβ-Gas6 (HA tag, Gla EGF delete, GE-/-) SEQ ID NO: 140
MAPSLSPGPAALRRAPQLLLLLLAAECALA(SS)
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPLTFGGGTKVEIKRGGGGSGGG
GSGGGGSEVQLVESGGGVVQPGRSLRLSCAASGFAFSSYGMHWVRQAPGKGLEWV
AVIWFDGTKKYYTDSVKGRFTISRDNSKNTLYLQMNTLRAEDTAVYYCARDRGIGA
RRGPYYMDVWGKGTTVTVSS (Adu-scFv)
GGGGSGGGGS (Linker)
DILPCVPFSVAKSVKSLYLGRMFSGTPVIRLRFKRLQPTRLVAEFDFRTFDPEGILLFA
GGHQDSTWIVLALRAGRLELQLRYNGVGRVTSSGPVINHGMWQTISVEELARNLVI
KVNRDAVMKIAVAGDLFQPERGLYHLNLTVGGIPFHEKDLVQPINPRLDGCMRSWN
WLNGEDTTIQETVKVNTRMQCFSVTERGSFYPGSGFAFYSLDYMRTPLDVGTESTW
EVEVVAHIRPAADTGVLFALWAPDLRAVPLSVALVDYHSTKKLKKQLVVLAVEHT
ALALMEIKVCDGQEHVVTVSLRDGEATLEVDGTRGQSEVSAAQLQERLAVLERHLR
SPVLTFAGGLPDVPVTSAPVTAFYRGCMTLEVNRRLLDLDEAAYKHSDITAHSCPPV
EPAAA (Gas6-Gla EGF deleted)
GSGSGSGSGSGSYPYDVPDYA* (HA)
6. Lentiviral Aducanumab IgG_IRES Zsgreen deleted SEQ ID NO: 141
MGWSCIILFLVATATG (SS)
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPLTFGGGTKVEIKRKRTVAAPSV
FIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC (Adu-Light chain)
RRKRGSGEGRGSLLTCGDVEENPGP (T2A)
MGWSCIILFLVATATG (SS)
EVQLVESGGGVVQPGRSLRLSCAASGFAFSSYGMHWVRQAPGKGLEWVAVIWFDG
TKKYYTDSVKGRFTISR
DNSKNTLYLQMNTLRAEDTAVYYCARDRGIGARRGPYYMDVWGKGTTVTVSSA
STKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSSDKTHTSPPCPA
PELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK* (Adu-
Heavy chain)
7. Endogenous full sequence human Gas6 protein SEQ ID NO: 142
MAPSLSPGPAALRRAPQLLLLLLAAECALAALLPAREATQFLRPRQRRAFQVFEEAK
QGHLERECVEELCSREEAREVFENDPETDYFYPRYLDCINKYGSPYTKNSGFATCVQ
NLPDQCTPNPCDRKGTQACQDLMGNFFCLCKAGWGGRLCDKDVNECSQENGGCL
QICHNKPGSFHCSCHSGFELSSDGRTCQDIDECADSEACGEARCKNLPGSYSCLCDE
GFAYSSQEKACRDVDECLQGRCEQVCVNSPGSYTCHCDGRGGLKLSQDMDTCEDIL
PCVPFSVAKSVKSLYLGRMFSGTPVIRLRFKRLQPTRLVAEFDFRTFDPEGILLFAGG
HQDSTWIVLALRAGRLELQLRYNGVGRVTSSGPVINHGMWQTISVEELARNLVIKV
NRDAVMKIAVAGDLFQPERGLYHLNLTVGGIPFHEKDLVQPINPRLDGCMRSWNW
LNGEDTTIQETVKVNTRMQCFSVTERGSFYPGSGFAFYSLDYMRTPLDVGTESTWE
VEVVAHIRPAADTGVLFALWAPDLRAVPLSVALVDYHSTKKLKKQLVVLAVEHTA
LALMEIKVCDGQEHVVTVSLRDGEATLEVDGTRGQSEVSAAQLQERLAVLERHLRS
PVLTFAGGLPDVPVTSAPVTAFYRGCMTLEVNRRLLDLDEAAYKHSDITAHSCPPVE
PAAA (full-length human Gas6)
QGSRADYKDHDGDYKDHDIDYKDDDDK* (FLAG)

TABLE 4
1. αAβ-Gas6(E) (FLAG tag, Gla delete, G-/-) SEQ ID NO: 143
ATGGCCCCTTCGCTCTCGCCCGGGCCCGCCGCCCTGCGCCGCGCGCCGCAGCTGC
TGCTGCTGCTGCTGGCCGCGGAGTGCGCGCTTGCC (SS)
GACATTCAGATGACTCAATCTCCTAGCTCTCTGAGCGCCTCCGTTGGAGATAGAG
TCACTATTACCTGCAGAGCCAGCCAATCCATCAGCTCTTATCTAAATTGGTACCA
ACAGAAGCCCGGCAAAGCGCCAAAGCTGCTCATCTACGCTGCAAGCTCCTTACA
GAGCGGAGTACCCAGCAGATTCTCAGGCAGTGGCAGTGGGACTGACTTCACATT
GACGATTAGCTCTCTGCAGCCTGAAGACTTTGCCACATACTATTGTCAGCAGAGC
TATAGCACCCCGCTGACGTTTGGAGGCGGAACTAAGGTGGAAATCAAGAGAGG
AGGCGGGGGCTCCGGCGGGGGTGGCTCGGGGGGAGGAGGCTCAGAGGTTCAGC
TTGTCGAGTCTGGGGGGGGAGTCGTTCAGCCAGGTAGAAGCCTCAGACTGAGCT
GTGCCGCAAGTGGGTTTGCTTTTTCATCTTACGGTATGCACTGGGTGAGACAGGC
TCCTGGCAAAGGACTCGAGTGGGTCGCTGTAATATGGTTCGATGGTACAAAGAA
ATACTATACCGATAGTGTGAAAGGAAGATTCACCATTTCACGAGACAACAGTAA
AAATACCTTGTACCTTCAGATGAACACCCTGAGAGCAGAAGACACAGCCGTGTA
CTACTGCGCCAGAGATAGAGGTATCGGAGCAAGGCGTGGTCCCTATTATATGGA
TGTGTGGGGGAAGGGAACAACAGTGACTGTGAGCTCT (Adu-scFv)
GGCGGGGGCGGCAGCGGCGGCGGTGGCAGC (Linker)
TGCATCAACAAGTATGGGTCTCCGTACACCAAAAACTCAGGCTTCGCCACCTGC
GTGCAAAACCTGCCTGACCAGTGCACGCCCAACCCCTGCGATAGGAAGGGGACC
CAAGCCTGCCAGGACCTCATGGGCAACTTCTTCTGCCTGTGTAAAGCTGGCTGGG
GGGGCCGGCTCTGCGACAAAGATGTCAACGAATGCAGCCAGGAGAACGGGGGC
TGCCTCCAGATCTGCCACAACAAGCCGGGTAGCTTCCACTGTTCCTGCCACAGCG
GCTTCGAGCTCTCCTCTGATGGCAGGACCTGCCAAGACATAGACGAGTGCGCAG
ACTCGGAGGCCTGCGGGGAGGCGCGCTGCAAGAACCTGCCCGGCTCCTACTCCT
GCCTCTGTGACGAGGGCTTTGCGTACAGCTCCCAGGAGAAGGCTTGCCGAGATG
TGGACGAGTGTCTGCAGGGCCGCTGTGAGCAGGTCTGCGTGAACTCCCCAGGGA
GCTACACCTGCCACTGTGACGGGCGTGGGGGCCTCAAGCTGTCCCAGGACATGG
ACACCTGTGAGGACATCTTGCCGTGCGTGCCCTTCAGCGTGGCCAAGAGTGTGA
AGTCCTTGTACCTGGGCCGGATGTTCAGTGGGACCCCCGTGATCCGACTGCGCTT
CAAGAGGCTGCAGCCCACCAGGCTGGTAGCTGAGTTTGACTTCCGGACCTTTGA
CCCCGAGGGCATCCTCCTCTTTGCCGGAGGCCACCAGGACAGCACCTGGATCGT
GCTGGCCCTGAGAGCCGGCCGGCTGGAGCTGCAGCTGCGCTACAACGGTGTCGG
CCGTGTCACCAGCAGCGGCCCGGTCATCAACCATGGCATGTGGCAGACAATCTC
TGTTGAGGAGCTGGCGCGGAATCTGGTCATCAAGGTCAACAGGGATGCTGTCAT
GAAAATCGCGGTGGCCGGGGACTTGTTCCAACCGGAGCGAGGACTGTATCATCT
GAACCTGACCGTGGGAGGTATTCCCTTCCATGAGAAGGACCTCGTGCAGCCTAT
AAACCCTCGTCTGGATGGCTGCATGAGGAGCTGGAACTGGCTGAACGGAGAAGA
CACCACCATCCAGGAAACGGTGAAAGTGAACACGAGGATGCAGTGCTTCTCGGT
GACGGAGAGAGGCTCTTTCTACCCCGGGAGCGGCTTCGCCTTCTACAGCCTGGA
CTACATGCGGACCCCTCTGGACGTCGGGACTGAATCAACCTGGGAAGTAGAAGT
CGTGGCTCACATCCGCCCAGCCGCAGACACAGGCGTGCTGTTTGCGCTCTGGGC
CCCCGACCTCCGTGCCGTGCCTCTCTCTGTGGCACTGGTAGACTATCACTCCACG
AAGAAACTCAAGAAGCAGCTGGTGGTCCTGGCCGTGGAGCATACGGCCTTGGCC
CTAATGGAGATCAAGGTCTGCGACGGCCAAGAGCACGTGGTCACCGTCTCGCTG
AGGGACGGTGAGGCCACCCTGGAGGTGGACGGCACCAGGGGCCAGAGCGAGGT
GAGCGCCGCGCAGCTGCAGGAGAGGCTGGCCGTGCTCGAGAGGCACCTGCGGA
GCCCCGTGCTCACCTTTGCTGGCGGCCTGCCAGATGTGCCGGTGACTTCAGCGCC
AGTCACCGCGTTCTACCGCGGCTGCATGACACTGGAGGTCAACCGGAGGCTGCT
GGACCTGGACGAGGCGGCGTACAAGCACAGCGACATCACGGCCCACTCCTGCCC
CCCCGTGGAGCCCGCCGCAGCC (Gas6-Gla deleted)
caagGATCCCGGGCTGACTACAAAGACCATGACGGTGATTATAAAGATCATGACA
TCGACTACAAGGATGACGATGACAAGtga (FLAG)
2. αFITC-Gas6(E) (FLAG tag, Gla delete, G-/-) SEQ ID NO: 144
ATGGCCCCTTCGCTCTCGCCCGGGCCCGCCGCCCTGCGCCGCGCGCCGCAGCTGC
TGCTGCTGCTGCTGGCCGCGGAGTGCGCGCTTGCC (SS)
CAGGTTCAGCTGGTTGAGAGCGGAGGCAATCTGGTTCAGCCCGGTGGTAGTCTG
CGTCTGTCTTGTGCGGCGTCAGGGTTCACTTTCGGTAGTTTTTCAATGAGCTGGG
TCCGTCAGGCACCAGGCGGTGGGCTGGAATGGGTGGCAGGTCTGTCTGCACGTA
GCTCCCTGACCCACTATGCAGATAGTGTTAAAGGGCGGTTCACAATTTCACGCG
ACAACGCTAAGAATAGCGTCTACCTGCAAATGAACTCCCTGCGGGTCGAGGATA
CCGCAGTGTATTACTGCGCTCGCCGTTCTTATGACTCTAGTGGATACTGGGGCCA
TTTTTATAGCTACATGGATGTGTGGGGACAGGGCACTCTGGTGACCGTTTCCGGA
GGCGGTGGGTCTGGAGGCGGTGGGAGTGGAGGCGGTGGGTCAAGCGTTCTGACC
CAGCCGTCCTCTGTCAGCGCCGCGCCAGGCCAGAAAGTGACAATTTCCTGTTCTG
GAAGTACTTCAAACATCGGCAACAATTATGTTTCCTGGTATCAGCAGCACCCGG
GCAAAGCGCCCAAGCTGATGATTTATGATGTGTCTAAACGTCCAAGTGGTGTTCC
TGACCGGTTCAGCGGTTCCAAGTCTGGGAATAGTGCCTCACTGGACATCTCAGG
CCTGCAAAGCGAAGATGAGGCGGACTATTACTGCGCAGCTTGGGATGACAGCCT
GTCCGAATTTCTGTTCGGCACCGGGACAAAGCTGACCGTGCTGGGC (aFITC-scFv)
GGCGGGGGCGGCAGCGGCGGCGGTGGCAGC (Linker)
TGCATCAACAAGTATGGGTCTCCGTACACCAAAAACTCAGGCTTCGCCACCTGC
GTGCAAAACCTGCCTGACCAGTGCACGCCCAACCCCTGCGATAGGAAGGGGACC
CAAGCCTGCCAGGACCTCATGGGCAACTTCTTCTGCCTGTGTAAAGCTGGCTGGG
GGGGCCGGCTCTGCGACAAAGATGTCAACGAATGCAGCCAGGAGAACGGGGGC
TGCCTCCAGATCTGCCACAACAAGCCGGGTAGCTTCCACTGTTCCTGCCACAGCG
GCTTCGAGCTCTCCTCTGATGGCAGGACCTGCCAAGACATAGACGAGTGCGCAG
ACTCGGAGGCCTGCGGGGAGGCGCGCTGCAAGAACCTGCCCGGCTCCTACTCCT
GCCTCTGTGACGAGGGCTTTGCGTACAGCTCCCAGGAGAAGGCTTGCCGAGATG
TGGACGAGTGTCTGCAGGGCCGCTGTGAGCAGGTCTGCGTGAACTCCCCAGGGA
GCTACACCTGCCACTGTGACGGGCGTGGGGGCCTCAAGCTGTCCCAGGACATGG
ACACCTGTGAGGACATCTTGCCGTGCGTGCCCTTCAGCGTGGCCAAGAGTGTGA
AGTCCTTGTACCTGGGCCGGATGTTCAGTGGGACCCCCGTGATCCGACTGCGCTT
CAAGAGGCTGCAGCCCACCAGGCTGGTAGCTGAGTTTGACTTCCGGACCTTTGA
CCCCGAGGGCATCCTCCTCTTTGCCGGAGGCCACCAGGACAGCACCTGGATCGT
GCTGGCCCTGAGAGCCGGCCGGCTGGAGCTGCAGCTGCGCTACAACGGTGTCGG
CCGTGTCACCAGCAGCGGCCCGGTCATCAACCATGGCATGTGGCAGACAATCTC
TGTTGAGGAGCTGGCGCGGAATCTGGTCATCAAGGTCAACAGGGATGCTGTCAT
GAAAATCGCGGTGGCCGGGGACTTGTTCCAACCGGAGCGAGGACTGTATCATCT
GAACCTGACCGTGGGAGGTATTCCCTTCCATGAGAAGGACCTCGTGCAGCCTAT
AAACCCTCGTCTGGATGGCTGCATGAGGAGCTGGAACTGGCTGAACGGAGAAGA
CACCACCATCCAGGAAACGGTGAAAGTGAACACGAGGATGCAGTGCTTCTCGGT
GACGGAGAGAGGCTCTTTCTACCCCGGGAGCGGCTTCGCCTTCTACAGCCTGGA
CTACATGCGGACCCCTCTGGACGTCGGGACTGAATCAACCTGGGAAGTAGAAGT
CGTGGCTCACATCCGCCCAGCCGCAGACACAGGCGTGCTGTTTGCGCTCTGGGC
CCCCGACCTCCGTGCCGTGCCTCTCTCTGTGGCACTGGTAGACTATCACTCCACG
AAGAAACTCAAGAAGCAGCTGGTGGTCCTGGCCGTGGAGCATACGGCCTTGGCC
CTAATGGAGATCAAGGTCTGCGACGGCCAAGAGCACGTGGTCACCGTCTCGCTG
AGGGACGGTGAGGCCACCCTGGAGGTGGACGGCACCAGGGGCCAGAGCGAGGT
GAGCGCCGCGCAGCTGCAGGAGAGGCTGGCCGTGCTCGAGAGGCACCTGCGGA
GCCCCGTGCTCACCTTTGCTGGCGGCCTGCCAGATGTGCCGGTGACTTCAGCGCC
AGTCACCGCGTTCTACCGCGGCTGCATGACACTGGAGGTCAACCGGAGGCTGCT
GGACCTGGACGAGGCGGCGTACAAGCACAGCGACATCACGGCCCACTCCTGCCC
CCCCGTGGAGCCCGCCGCAGCC (Gas6-Gla deleted)
caagGATCCCGGGCTGACTACAAAGACCATGACGGTGATTATAAAGATCATGACA
TCGACTACAAGGATGACGATGACAAGtga (FLAG)
3. αAβ-Gas6 (FLAG tag, Gla EGF delete, GE-/-) SEQ ID NO: 145
ATGGCCCCTTCGCTCTCGCCCGGGCCCGCCGCCCTGCGCCGCGCGCCGCAGCTGC
TGCTGCTGCTGCTGGCCGCGGAGTGCGCGCTTGCC (SS)
GACATTCAGATGACTCAATCTCCTAGCTCTCTGAGCGCCTCCGTTGGAGATAGAG
TCACTATTACCTGCAGAGCCAGCCAATCCATCAGCTCTTATCTAAATTGGTACCA
ACAGAAGCCCGGCAAAGCGCCAAAGCTGCTCATCTACGCTGCAAGCTCCTTACA
GAGCGGAGTACCCAGCAGATTCTCAGGCAGTGGCAGTGGGACTGACTTCACATT
GACGATTAGCTCTCTGCAGCCTGAAGACTTTGCCACATACTATTGTCAGCAGAGC
TATAGCACCCCGCTGACGTTTGGAGGCGGAACTAAGGTGGAAATCAAGAGAGG
AGGCGGGGGCTCCGGCGGGGGTGGCTCGGGGGGAGGAGGCTCAGAGGTTCAGC
TTGTCGAGTCTGGGGGGGGAGTCGTTCAGCCAGGTAGAAGCCTCAGACTGAGCT
GTGCCGCAAGTGGGTTTGCTTTTTCATCTTACGGTATGCACTGGGTGAGACAGGC
TCCTGGCAAAGGACTCGAGTGGGTCGCTGTAATATGGTTCGATGGTACAAAGAA
ATACTATACCGATAGTGTGAAAGGAAGATTCACCATTTCACGAGACAACAGTAA
AAATACCTTGTACCTTCAGATGAACACCCTGAGAGCAGAAGACACAGCCGTGTA
CTACTGCGCCAGAGATAGAGGTATCGGAGCAAGGCGTGGTCCCTATTATATGGA
TGTGTGGGGGAAGGGAACAACAGTGACTGTGAGCTCT (Adu-scFv)
GGCGGGGGCGGCAGCGGCGGCGGTGGCAGC (Linker)
GACATCTTGCCGTGCGTGCCCTTCAGCGTGGCCAAGAGTGTGAAGTCCTTGTACC
TGGGCCGGATGTTCAGTGGGACCCCCGTGATCCGACTGCGCTTCAAGAGGCTGC
AGCCCACCAGGCTGGTAGCTGAGTTTGACTTCCGGACCTTTGACCCCGAGGGCA
TCCTCCTCTTTGCCGGAGGCCACCAGGACAGCACCTGGATCGTGCTGGCCCTGAG
AGCCGGCCGGCTGGAGCTGCAGCTGCGCTACAACGGTGTCGGCCGTGTCACCAG
CAGCGGCCCGGTCATCAACCATGGCATGTGGCAGACAATCTCTGTTGAGGAGCT
GGCGCGGAATCTGGTCATCAAGGTCAACAGGGATGCTGTCATGAAAATCGCGGT
GGCCGGGGACTTGTTCCAACCGGAGCGAGGACTGTATCATCTGAACCTGACCGT
GGGAGGTATTCCCTTCCATGAGAAGGACCTCGTGCAGCCTATAAACCCTCGTCTG
GATGGCTGCATGAGGAGCTGGAACTGGCTGAACGGAGAAGACACCACCATCCA
GGAAACGGTGAAAGTGAACACGAGGATGCAGTGCTTCTCGGTGACGGAGAGAG
GCTCTTTCTACCCCGGGAGCGGCTTCGCCTTCTACAGCCTGGACTACATGCGGAC
CCCTCTGGACGTCGGGACTGAATCAACCTGGGAAGTAGAAGTCGTGGCTCACAT
CCGCCCAGCCGCAGACACAGGCGTGCTGTTTGCGCTCTGGGCCCCCGACCTCCGT
GCCGTGCCTCTCTCTGTGGCACTGGTAGACTATCACTCCACGAAGAAACTCAAG
AAGCAGCTGGTGGTCCTGGCCGTGGAGCATACGGCCTTGGCCCTAATGGAGATC
AAGGTCTGCGACGGCCAAGAGCACGTGGTCACCGTCTCGCTGAGGGACGGTGAG
GCCACCCTGGAGGTGGACGGCACCAGGGGCCAGAGCGAGGTGAGCGCCGCGCA
GCTGCAGGAGAGGCTGGCCGTGCTCGAGAGGCACCTGCGGAGCCCCGTGCTCAC
CTTTGCTGGCGGCCTGCCAGATGTGCCGGTGACTTCAGCGCCAGTCACCGCGTTC
TACCGCGGCTGCATGACACTGGAGGTCAACCGGAGGCTGCTGGACCTGGACGAG
GCGGCGTACAAGCACAGCGACATCACGGCCCACTCCTGCCCCCCCGTGGAGCCC
GCCGCAGCC (Gas6-Gla EGF deleted)
caaGGATCCCGGGCTGACTACAAAGACCATGACGGTGATTATAAAGATCATGACA
TCGACTACAAGGATGACGATGACAAGTGA (FLAG)
4. αFITC-Gas6 (FLAG tag, Gla EGF delete, GE-/-) SEQ ID NO: 146
ATGGCCCCTTCGCTCTCGCCCGGGCCCGCCGCCCTGCGCCGCGCGCCGCAGCTGC
TGCTGCTGCTGCTGGCCGCGGAGTGCGCGCTTGCC (SS)
CAGGTTCAGCTGGTTGAGAGCGGAGGCAATCTGGTTCAGCCCGGTGGTAGTCTG
CGTCTGTCTTGTGCGGCGTCAGGGTTCACTTTCGGTAGTTTTTCAATGAGCTGGG
TCCGTCAGGCACCAGGCGGTGGGCTGGAATGGGTGGCAGGTCTGTCTGCACGTA
GCTCCCTGACCCACTATGCAGATAGTGTTAAAGGGCGGTTCACAATTTCACGCG
ACAACGCTAAGAATAGCGTCTACCTGCAAATGAACTCCCTGCGGGTCGAGGATA
CCGCAGTGTATTACTGCGCTCGCCGTTCTTATGACTCTAGTGGATACTGGGGCCA
TTTTTATAGCTACATGGATGTGTGGGGACAGGGCACTCTGGTGACCGTTTCCGGA
GGCGGTGGGTCTGGAGGCGGTGGGAGTGGAGGCGGTGGGTCAAGCGTTCTGACC
CAGCCGTCCTCTGTCAGCGCCGCGCCAGGCCAGAAAGTGACAATTTCCTGTTCTG
GAAGTACTTCAAACATCGGCAACAATTATGTTTCCTGGTATCAGCAGCACCCGG
GCAAAGCGCCCAAGCTGATGATTTATGATGTGTCTAAACGTCCAAGTGGTGTTCC
TGACCGGTTCAGCGGTTCCAAGTCTGGGAATAGTGCCTCACTGGACATCTCAGG
CCTGCAAAGCGAAGATGAGGCGGACTATTACTGCGCAGCTTGGGATGACAGCCT
GTCCGAATTTCTGTTCGGCACCGGGACAAAGCTGACCGTGCTGGGC (aFITC-scFv)
GGCGGGGGCGGCAGCGGCGGCGGTGGCAGC (Linker)
GACATCTTGCCGTGCGTGCCCTTCAGCGTGGCCAAGAGTGTGAAGTCCTTGTACC
TGGGCCGGATGTTCAGTGGGACCCCCGTGATCCGACTGCGCTTCAAGAGGCTGC
AGCCCACCAGGCTGGTAGCTGAGTTTGACTTCCGGACCTTTGACCCCGAGGGCA
TCCTCCTCTTTGCCGGAGGCCACCAGGACAGCACCTGGATCGTGCTGGCCCTGAG
AGCCGGCCGGCTGGAGCTGCAGCTGCGCTACAACGGTGTCGGCCGTGTCACCAG
CAGCGGCCCGGTCATCAACCATGGCATGTGGCAGACAATCTCTGTTGAGGAGCT
GGCGCGGAATCTGGTCATCAAGGTCAACAGGGATGCTGTCATGAAAATCGCGGT
GGCCGGGGACTTGTTCCAACCGGAGCGAGGACTGTATCATCTGAACCTCACCGT
GGGAGGTATTCCCTTCCATGAGAAGGACCTCGTGCAGCCTATAAACCCTCGTCTG
GATGGCTGTATGAGGAGCTGGAACTGGCTGAACGGAGAAGACACCACCATCCA
GGAAACGGTGAAAGTGAACACGAGGATGCAGTGCTTCTCGGTGACGGAGAGAG
GCTCTTTCTACCCCGGGAGCGGCTTCGCCTTCTACAGCCTGGACTACATGCGGAC
CCCTCTGGACGTCGGGACTGAATCAACCTGGGAAGTAGAAGTCGTGGCTCACAT
CCGCCCAGCCGCAGACACAGGCGTGCTGTTTGCGCTCTGGGCCCCCGACCTCCGT
GCCGTGCCTCTCTCTGTGGCACTGGTAGACTATCACTCCACGAAGAAACTCAAG
AAGCAGCTGGTGGTCCTGGCCGTGGAGCATACGGCCTTGGCCCTAATGGAGATC
AAGGTCTGCGACGGCCAAGAGCACGTGGTCACCGTCTCGCTGAGGGACGGTGAG
GCCACCCTGGAGGTGGACGGCACCAGGGGCCAGAGCGAGGTGAGCGCCGCGCA
GCTGCAGGAGAGGCTGGCCGTGCTCGAGAGGCACCTGCGGAGCCCCGTGCTCAC
CTTTGCCGGCGGCCTGCCAGATGTGCCGGTGACTTCAGCGCCAGTCACCGCGTTC
TACCGCGGCTGCATGACACTGGAGGTCAACCGGAGGCTGCTGGACCTGGACGAG
GCGGCGTACAAGCACAGCGACATCACGGCCCACTCCTGCCCCCCCGTGGAGCCC
GCCGCAGCC (Gas6-Gla EGF deleted)
caaGGATCCCGGGCTGACTACAAAGACCATGACGGTGATTATAAAGATCATGACA
TCGACTACAAGGATGACGATGACAAGtga (FLAG)
5. αAβ-Gas6 HA tag (Gla EGF delete, GE-/-) SEQ ID NO: 147
ATGGCCCCTTCGCTCTCGCCCGGGCCCGCCGCCCTGCGCCGCGCGCCGCAGCTGC
TGCTGCTGCTGCTGGCCGCGGAGTGCGCGCTTGCC (SS)
GACATTCAGATGACTCAATCTCCTAGCTCTCTGAGCGCCTCCGTTGGAGATAGAG
TCACTATTACCTGCAGAGCCAGCCAATCCATCAGCTCTTATCTAAATTGGTACCA
ACAGAAGCCCGGCAAAGCGCCAAAGCTGCTCATCTACGCTGCAAGCTCCTTACA
GAGCGGAGTACCCAGCAGATTCTCAGGCAGTGGCAGTGGGACTGACTTCACATT
GACGATTAGCTCTCTGCAGCCTGAAGACTTTGCCACATACTATTGTCAGCAGAGC
TATAGCACCCCGCTGACGTTTGGAGGCGGAACTAAGGTGGAAATCAAGAGAGG
AGGCGGGGGCTCCGGCGGGGGTGGCTCGGGGGGAGGAGGCTCAGAGGTTCAGC
TTGTCGAGTCTGGGGGGGGAGTCGTTCAGCCAGGTAGAAGCCTCAGACTGAGCT
GTGCCGCAAGTGGGTTTGCTTTTTCATCTTACGGTATGCACTGGGTGAGACAGGC
TCCTGGCAAAGGACTCGAGTGGGTCGCTGTAATATGGTTCGATGGTACAAAGAA
ATACTATACCGATAGTGTGAAAGGAAGATTCACCATTTCACGAGACAACAGTAA
AAATACCTTGTACCTTCAGATGAACACCCTGAGAGCAGAAGACACAGCCGTGTA
CTACTGCGCCAGAGATAGAGGTATCGGAGCAAGGCGTGGTCCCTATTATATGGA
TGTGTGGGGGAAGGGAACAACAGTGACTGTGAGCTCT (Adu-scFv)
GGCGGGGGCGGCAGCGGCGGCGGTGGCAGC (Linker)
GACATCTTGCCGTGCGTGCCCTTCAGCGTGGCCAAGAGTGTGAAGTCCTTGTACC
TGGGCCGGATGTTCAGTGGGACCCCCGTGATCCGACTGCGCTTCAAGAGGCTGC
AGCCCACCAGGCTGGTAGCTGAGTTTGACTTCCGGACCTTTGACCCCGAGGGCA
TCCTCCTCTTTGCCGGAGGCCACCAGGACAGCACCTGGATCGTGCTGGCCCTGAG
AGCCGGCCGGCTGGAGCTGCAGCTGCGCTACAACGGTGTCGGCCGTGTCACCAG
CAGCGGCCCGGTCATCAACCATGGCATGTGGCAGACAATCTCTGTTGAGGAGCT
GGCGCGGAATCTGGTCATCAAGGTCAACAGGGATGCTGTCATGAAAATCGCGGT
GGCCGGGGACTTGTTCCAACCGGAGCGAGGACTGTATCATCTGAACCTGACCGT
GGGAGGTATTCCCTTCCATGAGAAGGACCTCGTGCAGCCTATAAACCCTCGTCTG
GATGGCTGCATGAGGAGCTGGAACTGGCTGAACGGAGAAGACACCACCATCCA
GGAAACGGTGAAAGTGAACACGAGGATGCAGTGCTTCTCGGTGACGGAGAGAG
GCTCTTTCTACCCCGGGAGCGGCTTCGCCTTCTACAGCCTGGACTACATGCGGAC
CCCTCTGGACGTCGGGACTGAATCAACCTGGGAAGTAGAAGTCGTGGCTCACAT
CCGCCCAGCCGCAGACACAGGCGTGCTGTTTGCGCTCTGGGCCCCCGACCTCCGT
GCCGTGCCTCTCTCTGTGGCACTGGTAGACTATCACTCCACGAAGAAACTCAAG
AAGCAGCTGGTGGTCCTGGCCGTGGAGCATACGGCCTTGGCCCTAATGGAGATC
AAGGTCTGCGACGGCCAAGAGCACGTGGTCACCGTCTCGCTGAGGGACGGTGAG
GCCACCCTGGAGGTGGACGGCACCAGGGGCCAGAGCGAGGTGAGCGCCGCGCA
GCTGCAGGAGAGGCTGGCCGTGCTCGAGAGGCACCTGCGGAGCCCCGTGCTCAC
CTTTGCTGGCGGCCTGCCAGATGTGCCGGTGACTTCAGCGCCAGTCACCGCGTTC
TACCGCGGCTGCATGACACTGGAGGTCAACCGGAGGCTGCTGGACCTGGACGAG
GCGGCGTACAAGCACAGCGACATCACGGCCCACTCCTGCCCCCCCGTGGAGCCC
GCCGCAGCC (Gas6-Gla EGF deleted)
GGCAGCGGCAGCGGCAGCGGCAGCGGCAGCGGCAGCTACCCATACGATGTTCC
AGATTACGCTTGA (HA)
6. Lentiviral Aducanumab IgG_IRES Zsgreen deleted SEQ ID NO: 148
GGATCCATGGGCTGGTCCTGCATCATCCTGTTCCTGGTGGCCACCGCCACCGGC
(SS)
GACATTCAGATGACTCAATCTCCTAGCTCTCTGAGCGCCTCCGTTGGAGATAGAG
TCACTATTACCTGCAGAGCCAGCCAATCCATCAGCTCTTATCTAAATTGGTACCA
ACAGAAGCCCGGCAAAGCGCCAAAGCTGCTCATCTACGCTGCAAGCTCCTTACA
GAGCGGAGTACCCAGCAGATTCTCAGGCAGTGGCAGTGGGACTGACTTCACATT
GACGATTAGCTCTCTGCAGCCTGAAGACTTTGCCACATACTATTGTCAGCAGAGC
TATAGCACCCCGCTGACGTTTGGAGGCGGAACTAAGGTGGAAATCAAGAGAAA
ACGAACTGTGGCTGCACCATCTGTCTTCATCTTCCCGCCATCTGATGAGCAGTTG
AAATCTGGAACTGCCTCTGTCGTGTGCCTGCTGAATAACTTCTATCCCAGAGAGG
CCAAAGTACAGTGGAAGGTGGATAACGCCCTCCAATCGGGTAACTCCCAGGAGA
GTGTCACAGAGCAGGACAGCAAGGACAGCACCTACAGCCTCAGCAGCACCCTG
ACGCTGAGCAAAGCAGACTACGAGAAACACAAAGTCTACGCCTGCGAAGTCAC
CCATCAGGGCCTGTCCTCGCCCGTCACAAAGAGCTTCAACAGGGGAGAGTGT
(Adu-Light chain)
CGCAGAAAACGCGGAAGCGGAGAGGGCAGAGGAAGTCTTCTAACATGCGGTGA
CGTGGAGGAGAATCCCGGCCCT (T2A)
ATGGGCTGGTCCTGCATCATCCTGTTCCTGGTGGCCACCGCCACCGGC (SS)
GAGGTTCAGCTTGTCGAGTCTGGGGGGGGAGTCGTTCAGCCAGGTAGAAGCCTC
AGACTGAGCTGTGCCGCAAGTGGGTTTGCTTTTTCATCTTACGGTATGCACTGGG
TGAGACAGGCTCCTGGCAAAGGACTCGAGTGGGTCGCTGTAATATGGTTCGATG
GTACAAAGAAATACTATACCGATAGTGTGAAAGGAAGATTCACCATTTCACGAG
ACAACAGTAAAAATACCTTGTACCTTCAGATGAACACCCTGAGAGCAGAAGACA
CAGCCGTGTACTACTGCGCCAGAGATAGAGGTATCGGAGCAAGGCGTGGTCCCT
ATTATATGGATGTGTGGGGGAAGGGAACAACAGTGACTGTGAGCTCTGCCTCCA
CCAAGGGCCCATCGGTCTTCCCCCTGGCACCCTCCTCCAAGAGCACCTCTGGGGG
CACAGCGGCCCTGGGCTGCCTGGTCAAGGACTACTTCCCCGAACCGGTGACGGT
GTCGTGGAACTCAGGCGCCCTGACCAGCGGCGTGCACACCTTCCCGGCTGTCCT
ACAGTCCTCAGGACTCTACTCCCTCAGCAGCGTGGTGACTGTGCCCTCTAGCAGC
TTGGGCACCCAGACCTACATCTGCAACGTGAATCACAAGCCCAGCAACACCAAG
GTGGACAAGAAAGTTGAGCCCAAATCTAGCGACAAAACTCACACAAGCCCACC
GTGCCCAGCACCTGAACTCCTGGGGGGACCGTCAGTCTTCCTCTTCCCCCCAAAA
CCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTG
GAGGTGCATAATGCCAAGACAAAGCCGCGGGAGGAGCAGTACAACAGCACGTA
CCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCAT
CTCCAAAGCCAAAGGGCAGCCCCGAGAACCACAGGTGTACACCCTGCCCCCATC
CCGGGATGAGCTGACCAAGAACCAGGTCAGCCTGACCTGCCTGGTCAAAGGCTT
CTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTACAG
CAAGCTCACCGTGGACAAGAGCAGGTGGCAGCAGGGGAACGTCTTCTCATGCTC
CGTGATGCATGAGGCTCTGCACAACCACTACACGCAGAAGAGCCTCTCCCTGTC
CCCGGGTAAAtga (Adu-Heavy chain)
7. Endogenous full sequence human Gas6 protein SEQ ID NO: 149
ATGGCCCCTTCGCTCTCGCCCGGGCCCGCCGCCCTGCGCCGCGCGCCGCAGCTGC
TGCTGCTGCTGCTGGCCGCGGAGTGCGCGCTTGCCGCGCTGTTGCCGGCGCGCG
AGGCCACGCAGTTCCTGCGGCCCAGGCAGCGCCGCGCCTTTCAGGTCTTCGAGG
AGGCCAAGCAGGGCCACCTGGAGAGGGAGTGCGTGGAGGAGCTGTGCAGCCGC
GAGGAGGCGCGGGAGGTGTTCGAGAACGACCCCGAGACGGATTATTTTTACCCA
AGATACTTAGACTGCATCAACAAGTATGGGTCTCCGTACACCAAAAACTCAGGC
TTCGCCACCTGCGTGCAAAACCTGCCTGACCAGTGCACGCCCAACCCCTGCGAT
AGGAAGGGGACCCAAGCCTGCCAGGACCTCATGGGCAACTTCTTCTGCCTGTGT
AAAGCTGGCTGGGGGGGCCGGCTCTGCGACAAAGATGTCAACGAATGCAGCCA
GGAGAACGGGGGCTGCCTCCAGATCTGCCACAACAAGCCGGGTAGCTTCCACTG
TTCCTGCCACAGCGGCTTCGAGCTCTCCTCTGATGGCAGGACCTGCCAAGACATA
GACGAGTGCGCAGACTCGGAGGCCTGCGGGGAGGCGCGCTGCAAGAACCTGCC
CGGCTCCTACTCCTGCCTCTGTGACGAGGGCTTTGCGTACAGCTCCCAGGAGAAG
GCTTGCCGAGATGTGGACGAGTGTCTGCAGGGCCGCTGTGAGCAGGTCTGCGTG
AACTCCCCAGGGAGCTACACCTGCCACTGTGACGGGCGTGGGGGCCTCAAGCTG
TCCCAGGACATGGACACCTGTGAGGACATCTTGCCGTGCGTGCCCTTCAGCGTG
GCCAAGAGTGTGAAGTCCTTGTACCTGGGCCGGATGTTCAGTGGGACCCCCGTG
ATCCGACTGCGCTTCAAGAGGCTGCAGCCCACCAGGCTGGTAGCTGAGTTTGAC
TTCCGGACCTTTGACCCCGAGGGCATCCTCCTCTTTGCCGGAGGCCACCAGGACA
GCACCTGGATCGTGCTGGCCCTGAGAGCCGGCCGGCTGGAGCTGCAGCTGCGCT
ACAACGGTGTCGGCCGTGTCACCAGCAGCGGCCCGGTCATCAACCATGGCATGT
GGCAGACAATCTCTGTTGAGGAGCTGGCGCGGAATCTGGTCATCAAGGTCAACA
GGGATGCTGTCATGAAAATCGCGGTGGCCGGGGACTTGTTCCAACCGGAGCGAG
GACTGTATCATCTGAACCTGACCGTGGGAGGTATTCCCTTCCATGAGAAGGACCT
CGTGCAGCCTATAAACCCTCGTCTGGATGGCTGCATGAGGAGCTGGAACTGGCT
GAACGGAGAAGACACCACCATCCAGGAAACGGTGAAAGTGAACACGAGGATGC
AGTGCTTCTCGGTGACGGAGAGAGGCTCTTTCTACCCCGGGAGCGGCTTCGCCTT
CTACAGCCTGGACTACATGCGGACCCCTCTGGACGTCGGGACTGAATCAACCTG
GGAAGTAGAAGTCGTGGCTCACATCCGCCCAGCCGCAGACACAGGCGTGCTGTT
TGCGCTCTGGGCCCCCGACCTCCGTGCCGTGCCTCTCTCTGTGGCACTGGTAGAC
TATCACTCCACGAAGAAACTCAAGAAGCAGCTGGTGGTCCTGGCCGTGGAGCAT
ACGGCCTTGGCCCTAATGGAGATCAAGGTCTGCGACGGCCAAGAGCACGTGGTC
ACCGTCTCGCTGAGGGACGGTGAGGCCACCCTGGAGGTGGACGGCACCAGGGG
CCAGAGCGAGGTGAGCGCCGCGCAGCTGCAGGAGAGGCTGGCCGTGCTCGAGA
GGCACCTGCGGAGCCCCGTGCTCACCTTTGCTGGCGGCCTGCCAGATGTGCCGGT
GACTTCAGCGCCAGTCACCGCGTTCTACCGCGGCTGCATGACACTGGAGGTCAA
CCGGAGGCTGCTGGACCTGGACGAGGCGGCGTACAAGCACAGCGACATCACGG
CCCACTCCTGCCCCCCCGTGGAGCCCGCCGCAGCC (full-length human Gas6)
caaGGATCCCGGGCTGACTACAAAGACCATGACGGTGATTATAAAGATCATGACA
TCGACTACAAGGATGACGATGACAAGTGA (FLAG)

### Preparation Example 2. Gas6-Based Fusion Molecule Targeting Tau

To prepare a tan-specific chimeric phagocytosis inducer based on Gas6 protein, the Gla domain and the EGF repeat domain were first removed, and a single-chain variable fragment (scFv) of semorinemab, a tau-specific antibody fragment; scFv), was introduced at that position (αTau-Gas6). Table 5 below shows the amino acid sequence and nucleotide sequence of the chimeric phagocytosis inducer.

TABLE 5
1. αTau-Gas6 (Tau-VL-G4Sx3-VH-LG-HA-T2A-EGFP, amino acid
sequence) SEQ ID NO: 150
MAPSLSPGPAALRRAPQLLLLLLAAECALA (SS)
DDVLTQTPLSLPVTPGQPASISCRSSQSIVHSNGNTYLEWYLQKPGQSPQLLIYKVSN
RFSGVPDRFSGSGSGTDFTLKISRVEAEDVGVYYCFQGSLVPWTFGQGTKVEIKGGG
GSGGGGSGGGGSEVQLVESGGGLVQPGGSLRLSCAASGLIFRSYGMSWVRQAPGKG
LEWVATINSGGTYTYYPDSVKGRFTISRDNSKNTLYLQMNSLRAEDTAVYYCANSYS
GAMDYWGQGTLVTVSS (aTau-scFv)
GGGGSGGGGS (Linker)
DILPCVPFSVAKSVKSLYLGRMFSGTPVIRLRFKRLQPTRLVAEFDFRTFDPEGILLFAG
GHQDSTWIVLALRAGRLELQLRYNGVGRVTSSGPVINHGMWQTISVEELARNLVIK
VNRDAVMKIAVAGDLFQPERGLYHLNLTVGGIPFHEKDLVQPINPRLDGCMRSWNW
LNGEDTTIQETVKVNTRMQCFSVTERGSFYPGSGFAFYSLDYMRTPLDVGTESTWEV
EVVAHIRPAADTGVLFALWAPDLRAVPLSVALVDYHSTKKLKKQLVVLAVEHTALAL
MEIKVCDGQEHVVTVSLRDGEATLEVDGTRGQSEVSAAQLQERLAVLERHLRSPVL
TFAGGLPDVPVTSAPVTAFYRGCMTLEVNRRLLDLDEAAYKHSDITAHSCPPVEPAA
A (Gas6-Gla EGF deleted)
GSGSGSGSGSGSYPYDVPDYA (HA)
EGRGSLLTCGDVEENPGP (T2A)
VSKGEELFTGVVPILVELDGDVNGHKFSVSGEGEGDATYGKLTLKFICTTGKLPVPW
PTLVTTLTYGVQCFSRYPDHMKQHDFFKSAMPEGYVQERTIFFKDDGNYKTRAEVK
FEGDTLVNRIELKGIDFKEDGNILGHKLEYNYNSHNVYIMADKQKNGIKVNFKIRHN
IEDGSVQLADHYQQNTPIGDGPVLLPDNHYLSTQSALSKDPNEKRDHMVLLEFVTA
AGITLGMDELYK*(EGFP)
2. αTau-Gas6 (Tau-VL-G4Sx3-VH-LG-HA-T2A-EGFP, nucleotide
sequence) SEQ ID NO: 151
ATGGCCCCTTCGCTCTCGCCCGGGCCCGCCGCCCTGCGCCGCGCGCCGCAGCTGC
TGCTGCTGCTGCTGGCCGCGGAGTGCGCGCTTGCC (SS)
GACGATGTATTAACACAAACTCCCCTATCATTGCCGGTGACCCCGGGCCAACCA
GCTTCGATCAGCTGCCGTAGCTCTCAGAGCATCGTGCACAGCAACGGTAATACC
TACCTGGAATGGTATTTGCAAAAACCGGGTCAATCCCCGCAGTTGCTGATTTATA
AAGTTTCGAATCGTTTCAGCGGTGTTCCGGATCGTTTCAGCGGCTCTGGCTCCGG
CACCGATTTTACGCTGAAGATCAGTCGCGTGGAAGCGGAGGACGTGGGTGTCTA
CTACTGCTTTCAGGGTAGTTTGGTGCCGTGGACCTTTGGTCAGGGTACTAAGGTG
GAAATTAAGGGTGGTGGGGGATCAGGTGGCGGCGGCAGCGGCGGTGGCGGGAG
CGAGGTACAACTAGTTGAATCAGGTGGAGGGTTGGTTCAGCCAGGTGGTTCGCT
GCGTCTGAGTTGTGCGGCAAGCGGTTTGATCTTTCGCAGCTATGGTATGAGCTGG
GTTCGTCAGGCGCCGGGCAAGGGTCTGGAGTGGGTGGCGACCATTAACTCTGGC
GGCACGTACACCTACTATCCCGACTCCGTGAAAGGCCGTTTCACCATCTCCCGCG
ACAATAGCAAAAACACCCTGTATTTGCAGATGAACTCGCTCCGCGCAGAGGACA
CCGCTGTGTACTACTGCGCCAATTCCTACAGCGGTGCTATGGATTATTGGGGTCA
GGGCACATTGGTGACTGTAAGCAGC (aTau-scFv)
GGCGGGGGCGGCAGCGGCGGCGGTGGCAGC (Linker)
GACATCTTGCCGTGCGTGCCCTTCAGCGTGGCCAAGAGTGTGAAGTCCTTGTACC
TGGGCCGGATGTTCAGTGGGACCCCCGTGATCCGACTGCGCTTCAAGAGGCTGC
AGCCCACCAGGCTGGTAGCTGAGTTTGACTTCCGGACCTTTGACCCCGAGGGCA
TCCTCCTCTTTGCCGGAGGCCACCAGGACAGCACCTGGATCGTGCTGGCCCTGAG
AGCCGGCCGGCTGGAGCTGCAGCTGCGCTACAACGGTGTCGGCCGTGTCACCAG
CAGCGGCCCGGTCATCAACCATGGCATGTGGCAGACAATCTCTGTTGAGGAGCT
GGCGCGGAATCTGGTCATCAAGGTCAACAGGGATGCTGTCATGAAAATCGCGGT
GGCCGGGGACTTGTTCCAACCGGAGCGAGGACTGTATCATCTGAACCTGACCGT
GGGAGGTATTCCCTTCCATGAGAAGGACCTCGTGCAGCCTATAAACCCTCGTCTG
GATGGCTGCATGAGGAGCTGGAACTGGCTGAACGGAGAAGACACCACCATCCA
GGAAACGGTGAAAGTGAACACGAGGATGCAGTGCTTCTCGGTGACGGAGAGAG
GCTCTTTCTACCCCGGGAGCGGCTTCGCCTTCTACAGCCTGGACTACATGCGGAC
CCCTCTGGACGTCGGGACTGAATCAACCTGGGAAGTAGAAGTCGTGGCTCACAT
CCGCCCAGCCGCAGACACAGGCGTGCTGTTTGCGCTCTGGGCCCCCGACCTCCGT
GCCGTGCCTCTCTCTGTGGCACTGGTAGACTATCACTCCACGAAGAAACTCAAG
AAGCAGCTGGTGGTCCTGGCCGTGGAGCATACGGCCTTGGCCCTAATGGAGATC
AAGGTCTGCGACGGCCAAGAGCACGTGGTCACCGTCTCGCTGAGGGACGGTGAG
GCCACCCTGGAGGTGGACGGCACCAGGGGCCAGAGCGAGGTGAGCGCCGCGCA
GCTGCAGGAGAGGCTGGCCGTGCTCGAGAGGCACCTGCGGAGCCCCGTGCTCAC
CTTTGCTGGCGGCCTGCCAGATGTGCCGGTGACTTCAGCGCCAGTCACCGCGTTC
TACCGCGGCTGCATGACACTGGAGGTCAACCGGAGGCTGCTGGACCTGGACGAG
GCGGCGTACAAGCACAGCGACATCACGGCCCACTCCTGCCCCCCCGTGGAGCCC
GCCGCAGCC (Gas6-Gla EGF deleted)
GGCAGCGGCAGCGGCAGCGGCAGCGGCAGCGGCAGCtacccatacgatgttcca
gattacgct (HA)
GAGGGCAGAGGAAGTCTGCTAACATGCGGTGACGTCGAGGAGAATCCTGGCCC
A (T2A)
GTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGA
TGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGC
CGCTACCCCGACCACATGAAGCAGCACGACTTCTTCAAGTCCGCCATGCCCGAA
GGCTACGTCCAGGAGCGCACCATCTTCTTCAAGGACGACGGCAACTACAAGACC
CGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAA
CTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGA
CCACTACCAGCAGAACACCCCCATCGGCGACGGCCCCGTGCTGCTGCCCGACAA
CCACTACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCATGGAC
GAGCTGTACAAGtaa (EGFP)

### Preparation Example 3. Gas6-Based Fusion Molecule Targeting Alpha-Synuclein

To prepare an alpha-synuclein-specific chimera phagocytosis inducer based on Gas6 protein, the Gla domain and the EGF repeat domain were first removed, and a single-chain variable region (scFv) of cinpanemab, an alpha-synuclein-specific antibody, was introduced at that position (ααSyn-Gas6). Table 6 below shows the amino acid sequence and nucleotide sequence of the chimeric phagocytosis inducer.

TABLE 6
1. ααSyn-Gas6 (Cinpanemab (aSyn)_VL-G4Sx3-VH-LG-HA-T2A-EGFP,
amino acid sequence) SEQ ID NO: 152
MAPSLSPGPAALRRAPQLLLLLLAAECALA (SS)
SYELTQPPSVSVSPGQTARITCSGEALPMQFAHWYQQRPGKAPVIVVYKDSERPSGV
PERFSGSSSGTTATLTITGVQAEDEADYYCQSPDSTNTYEVFGGGTKLTVLGGGGSG
GGGSGGGGSEVQLVESGGGLVEPGGSLRLSCAVSGFDFEKAWMSWVRQAPGQGLQ
WVARIKSTADGGTTSYAAPVEGRFIISRDDSRNMLYLQMNSLKTEDTAVYYCTSAH
WGQGTLVTVSS (aaSyn-scFv)
GGGGSGGGGS (Linker)
DILPCVPFSVAKSVKSLYLGRMFSGTPVIRLRFKRLQPTRLVAEFDFRTFDPEGILLFA
GGHQDSTWIVLALRAGRLELQLRYNGVGRVTSSGPVINHGMWQTISVEELARNLVI
KVNRDAVMKIAVAGDLFQPERGLYHLNLTVGGIPFHEKDLVQPINPRLDGCMRSWN
WLNGEDTTIQETVKVNTRMQCFSVTERGSFYPGSGFAFYSLDYMRTPLDVGTESTW
EVEVVAHIRPAADTGVLFALWAPDLRAVPLSVALVDYHSTKKLKKQLVVLAVEHT
ALALMEIKVCDGQEHVVTVSLRDGEATLEVDGTRGQSEVSAAQLQERLAVLERHLR
SPVLTFAGGLPDVPVTSAPVTAFYRGCMTLEVNRRLLDLDEAAYKHSDITAHSCPPV
EPAAA (Gas6-Gla EGF deleted)
GSGSGSGSGSGSYPYDVPDYA (HA)
EGRGSLLTCGDVEENPGP (T2A)
VSKGEELFTGVVPILVELDGDVNGHKFSVSGEGEGDATYGKLTLKFICTTGKLPVPW
PTLVTTLTYGVQCFSRYPDHMKQHDFFKSAMPEGYVQERTIFFKDDGNYKTRAEVK
FEGDTLVNRIELKGIDFKEDGNILGHKLEYNYNSHNVYIMADKQKNGIKVNFKIRHN
IEDGSVQLADHYQQNTPIGDGPVLLPDNHYLSTQSALSKDPNEKRDHMVLLEFVTA
AGITLGMDELYK*
2. ααSyn-Gas6 (Cinpanemab (aSyn)_VL-G4Sx3-VH-LG-HA-T2A-EGFP,
nucleotide sequence) SEQ ID NO: 153
ATGGCCCCTTCGCTCTCGCCCGGGCCCGCCGCCCTGCGCCGCGCGCCGCAGCTGC
TGCTGCTGCTGCTGGCCGCGGAGTGCGCGCTTGCC (SS)
TCCTATGAGCTGACTCAGCCACCCTCGGTGTCAGTGTCCCCAGGACAGACGGCC
AGGATCACCTGCTCTGGAGAAGCATTGCCAATGCAATTTGCTCATTGGTACCAAC
AGAGGCCAGGCAAGGCCCCAGTGATAGTGGTGTACAAAGACAGTGAGAGACCG
TCAGGTGTCCCTGAGCGATTCTCTGGCTCCAGCTCAGGGACAACAGCCACGTTG
ACCATCACTGGAGTCCAGGCAGAAGATGAGGCTGACTATTACTGCCAGTCGCCA
GACAGCACTAACACTTATGAAGTCTTCGGCGGAGGGACCAAGCTGACCGTCCTA
GGTGGTGGGGGATCAGGTGGCGGCGGCAGCGGCGGTGGCGGGAGCGAGGTGCA
GCTGGTGGAGTCTGGGGGAGGTCTGGTCGAGCCGGGGGGGTCCCTAAGACTCTC
CTGTGCAGTCTCCGGATTCGATTTCGAAAAAGCCTGGATGAGTTGGGTCCGCCA
GGCTCCAGGGCAGGGGCTACAGTGGGTTGCCCGTATCAAGAGCACAGCTGATGG
TGGGACAACAAGCTACGCCGCCCCCGTGGAAGGCAGGTTCATCATCTCAAGAGA
TGATTCGAGAAACATGCTTTATCTGCAAATGAACAGTCTGAAAACTGAAGACAC
AGCCGTCTATTATTGTACATCAGCCCACTGGGGCCAGGGAACCCTGGTCACCGTC
TCCTCG (aaSyn-scFv)
GGCGGGGGCGGCAGCGGCGGCGGTGGCAGC (Linker)
GACATCTTGCCGTGCGTGCCCTTCAGCGTGGCCAAGAGTGTGAAGTCCTTGTACC
TGGGCCGGATGTTCAGTGGGACCCCCGTGATCCGACTGCGCTTCAAGAGGCTGC
AGCCCACCAGGCTGGTAGCTGAGTTTGACTTCCGGACCTTTGACCCCGAGGGCA
TCCTCCTCTTTGCCGGAGGCCACCAGGACAGCACCTGGATCGTGCTGGCCCTGAG
AGCCGGCCGGCTGGAGCTGCAGCTGCGCTACAACGGTGTCGGCCGTGTCACCAG
CAGCGGCCCGGTCATCAACCATGGCATGTGGCAGACAATCTCTGTTGAGGAGCT
GGCGCGGAATCTGGTCATCAAGGTCAACAGGGATGCTGTCATGAAAATCGCGGT
GGCCGGGGACTTGTTCCAACCGGAGCGAGGACTGTATCATCTGAACCTGACCGT
GGGAGGTATTCCCTTCCATGAGAAGGACCTCGTGCAGCCTATAAACCCTCGTCTG
GATGGCTGCATGAGGAGCTGGAACTGGCTGAACGGAGAAGACACCACCATCCA
GGAAACGGTGAAAGTGAACACGAGGATGCAGTGCTTCTCGGTGACGGAGAGAG
GCTCTTTCTACCCCGGGAGCGGCTTCGCCTTCTACAGCCTGGACTACATGCGGAC
CCCTCTGGACGTCGGGACTGAATCAACCTGGGAAGTAGAAGTCGTGGCTCACAT
CCGCCCAGCCGCAGACACAGGCGTGCTGTTTGCGCTCTGGGCCCCCGACCTCCGT
GCCGTGCCTCTCTCTGTGGCACTGGTAGACTATCACTCCACGAAGAAACTCAAG
AAGCAGCTGGTGGTCCTGGCCGTGGAGCATACGGCCTTGGCCCTAATGGAGATC
AAGGTCTGCGACGGCCAAGAGCACGTGGTCACCGTCTCGCTGAGGGACGGTGAG
GCCACCCTGGAGGTGGACGGCACCAGGGGCCAGAGCGAGGTGAGCGCCGCGCA
GCTGCAGGAGAGGCTGGCCGTGCTCGAGAGGCACCTGCGGAGCCCCGTGCTCAC
CTTTGCTGGCGGCCTGCCAGATGTGCCGGTGACTTCAGCGCCAGTCACCGCGTTC
TACCGCGGCTGCATGACACTGGAGGTCAACCGGAGGCTGCTGGACCTGGACGAG
GCGGCGTACAAGCACAGCGACATCACGGCCCACTCCTGCCCCCCCGTGGAGCCC
GCCGCAGCC (Gas6-Gla EGF deleted)
GGCAGCGGCAGCGGCAGCGGCAGCGGCAGCGGCAGCTACCCATACGATGTTCC
AGATTACGCT (HA)
GAGGGCAGAGGAAGTCTGCTAACATGCGGTGACGTCGAGGAGAATCCTGGCCC
A (T2A)
GTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGA
TGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGC
CGCTACCCCGACCACATGAAGCAGCACGACTTCTTCAAGTCCGCCATGCCCGAA
GGCTACGTCCAGGAGCGCACCATCTTCTTCAAGGACGACGGCAACTACAAGACC
CGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAA
CTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGA
CCACTACCAGCAGAACACCCCCATCGGCGACGGCCCCGTGCTGCTGCCCGACAA
CCACTACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCATGGAC
GAGCTGTACAAGTAA (EGFP)

### Preparation Example 4. ProS1-Based Fusion Molecule Targeting Beta-Amyloid

To prepare a beta-amyloid (Aβ3)-specific chimeric phagocytosis inducer based on ProS1 protein, the Gla domain and the EGF repeat domain were first removed, and a single-chain variable fragment (scFv) of aducanumab, a beta-amyloid-specific antibody, was introduced at that position (αAβ-ProS1). Table 7 below shows the amino acid sequence and nucleotide sequence of the chimeric phagocytosis inducer.

TABLE 7
1. αAβ-ProS1 (αAβ-ProS1(GE-)-FLAG, amino acid sequence)
SEQ ID NO: 154
MRVLGGRCGALLACLLLVLPVSEA (SS)
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPLTFGGGTKVEIKRGGGGSGGG
GSGGGGSEVQLVESGGGVVQPGRSLRLSCAASGFAFSSYGMHWVRQAPGKGLEWV
AVIWFDGTKKYYTDSVKGRFTISRDNSKNTLYLQMNTLRAEDTAVYYCARDRGIGA
RRGPYYMDVWGKGTTVTVSS (Adu-scFv)
GGGGSGGGGS (Linker)
VVSVCLPLNLDTKYELLYLAEQFAGVVLYLKFRLPEISRFSAEFDFRTYDSEGVILYA
ESIDHSAWLLIALRGGKIEVQLKNEHTSKITTGGDVINNGLWNMVSVEELEHSISIKIA
KEAVMDINKPGPLFKPENGLLETKVYFAGFPRKVESELIKPINPRLDGCIRSWNLMK
QGASGIKEIIQEKQNKHCLVTVEKGSYYPGSGIAQFHIDYNNVSSAEGWHVNVTLNI
RPSTGTGVMLALVSGNNTVPFAVSLVDSTSEKSQDILLSVENTVIYRIQALSLCSDQQ
SHLEFRVNRNNLELSTPLKIETISHEDLQRQLAVLDKAMKAKVATYLGGLPDVPFSA
TPVNAFYNGCMEVNINGVQLDLDEAISKHNDIRAHSCPSVWKKTKNS (ProS1(GE-))
QGSRADYKDHDGDYKDHDIDYKDDDDK* (FLAG)
2. αAβ-ProS1 (αAβ-ProS1(GE-)-FLAG, nucleotide sequence)
SEQ ID NO: 155
ATGAGGGTCCTGGGTGGGCGCTGCGGGGCGCTGCTGGCGTGTCTCCTCCTAGTG
CTTCCCGTCTCAGAGGCA (SS)
GACATTCAGATGACTCAATCTCCTAGCTCTCTGAGCGCCTCCGTTGGAGATAGAG
TCACTATTACCTGCAGAGCCAGCCAATCCATCAGCTCTTATCTAAATTGGTACCA
ACAGAAGCCCGGCAAAGCGCCAAAGCTGCTCATCTACGCTGCAAGCTCCTTACA
GAGCGGAGTACCCAGCAGATTCTCAGGCAGTGGCAGTGGGACTGACTTCACATT
GACGATTAGCTCTCTGCAGCCTGAAGACTTTGCCACATACTATTGTCAGCAGAGC
TATAGCACCCCGCTGACGTTTGGAGGCGGAACTAAGGTGGAAATCAAGAGAGG
AGGCGGGGGCTCCGGCGGGGGTGGCTCGGGGGGAGGAGGCTCAGAGGTTCAGC
TTGTCGAGTCTGGGGGGGGAGTCGTTCAGCCAGGTAGAAGCCTCAGACTGAGCT
GTGCCGCAAGTGGGTTTGCTTTTTCATCTTACGGTATGCACTGGGTGAGACAGGC
TCCTGGCAAAGGACTCGAGTGGGTCGCTGTAATATGGTTCGATGGTACAAAGAA
ATACTATACCGATAGTGTGAAAGGAAGATTCACCATTTCACGAGACAACAGTAA
AAATACCTTGTACCTTCAGATGAACACCCTGAGAGCAGAAGACACAGCCGTGTA
CTACTGCGCCAGAGATAGAGGTATCGGAGCAAGGCGTGGTCCCTATTATATGGA
TGTGTGGGGGAAGGGAACAACAGTGACTGTGAGCTCT (Adu-scFv)
GGCGGGGGCGGCAGCGGCGGCGGTGGCAGC (Linker)
GTTGTTTCAGTGTGCCTTCCCTTGAACCTTGACACAAAGTATGAATTACTTTACTT
GGCGGAGCAGTTTGCAGGGGTTGTTTTATATTTAAAATTTCGTTTGCCAGAAATC
AGCAGATTTTCAGCAGAATTTGATTTCCGGACATATGATTCAGAAGGCGTGATA
CTGTACGCAGAATCTATCGATCACTCAGCGTGGCTCCTGATTGCACTTCGTGGTG
GAAAGATTGAAGTTCAGCTTAAGAATGAACATACATCCAAAATCACAACTGGAG
GTGATGTTATTAATAATGGTCTATGGAATATGGTGTCTGTGGAAGAATTAGAAC
ATAGTATTAGCATTAAAATAGCTAAAGAAGCTGTGATGGATATAAATAAACCTG
GACCCCTTTTTAAGCCGGAAAATGGATTGCTGGAAACCAAAGTATACTTTGCAG
GATTCCCTCGGAAAGTGGAAAGTGAACTCATTAAACCGATTAACCCTCGTCTAG
ATGGATGTATACGAAGCTGGAATTTGATGAAGCAAGGAGCTTCTGGAATAAAGG
AAATTATTCAAGAAAAACAAAATAAGCATTGCCTGGTTACTGTGGAGAAGGGCT
CCTACTATCCTGGTTCTGGAATTGCTCAATTTCACATAGATTATAATAATGTATC
CAGTGCTGAGGGTTGGCATGTAAATGTGACCTTGAATATTCGTCCATCCACGGGC
ACTGGTGTTATGCTTGCCTTGGTTTCTGGTAACAACACAGTGCCCTTTGCTGTGTC
CTTGGTGGACTCCACCTCTGAAAAATCACAGGATATTCTGTTATCTGTTGAAAAT
ACTGTAATATATCGGATACAGGCCCTAAGTCTATGTTCCGATCAACAATCTCATC
TGGAATTTAGAGTCAACAGAAACAATCTGGAGTTGTCGACACCACTTAAAATAG
AAACCATCTCCCATGAAGACCTTCAAAGACAACTTGCCGTCTTGGACAAAGCAA
TGAAAGCAAAAGTGGCCACATACCTGGGTGGCCTTCCAGATGTTCCATTCAGTG
CCACACCAGTGAATGCCTTTTATAATGGCTGCATGGAAGTGAATATTAATGGTGT
ACAGTTGGATCTGGATGAAGCCATTTCTAAACATAATGATATTAGAGCTCACTCA
TGTCCATCAGTTTGGAAAAAGACAAAGAATTCT (ProS1(GE-))
CAAGGATCCCGGGCTGACTACAAAGACCATGACGGTGATTATAAAGATCATGAC
ATCGACTACAAGGATGACGATGACAAGtga (FLAG)

### Preparation Example 5. Gas6-Based Fusion Molecules Targeting Beta-Amyloid (II): Beta-Amyloid-Binding Regions in the Form of Fab or Mab

To prepare gas6 protein-based beta-amyloid Ah)-specific chimera phagocytosis inducer, the Gla domain, which recognizes PS (1phosphatidylserine) in apoptotic cells, was first removed, and an antigen-binding fragment (Fab) or monoclonal antibody (Mab) of the beta-amyloid-specific antibody aducanumab was introduces at that position (αAβ[Fab]-Gas6, and αAβ[Mab]-Gas6). Tables 8-10 below show the amino acid sequences and nucleotide sequences of the two chimeric phagocytosis inducers.

TABLE 8
Second Region Light Chain (SEQ ID NO: 161) (the light
chain which is capable of specifically binding to beta-
amyloid can form a dimer with the peptide of sequence of
SEQ ID NO: 156 or SEQ ID NO: 158 to form Fab Adu-Gas6
or Mab Adu-Gas6, respectively):
MGWSCIILFLVATATG (SS)
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPLTFGGGTKVEIKRKRTVAAPSV
FIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC*(Adu Light Chain)
1. αAβ[Fab]-Gas6 (Aducanumab VH-CH1 (Fab)-Gas6-FLAG, amino
acid sequence) SEQ ID NO: 156
METDTLLLWVLLLWVPGSTGD (SS)
EVQLVESGGGVVQPGRSLRLSCAASGFAFSSYGMHWVRQAPGKGLEWVAVIWFDG
TKKYYTDSVKGRFTISRDNSKNTLYLQMNTLRAEDTAVYYCARDRGIGARRGPYY
MDVWGKGTTVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVE
PKSCDKTH (Adu-VH-CH1)
GGGGSGGGGS (Linker)
DILPCVPFSVAKSVKSLYLGRMFSGTPVIRLRFKRLQPTRLVAEFDFRTFDPEGILLFA
GGHQDSTWIVLALRAGRLELQLRYNGVGRVTSSGPVINHGMWQTISVEELARNLVI
KVNRDAVMKIAVAGDLFQPERGLYHLNLTVGGIPFHEKDLVQPINPRLDGCMRSWN
WLNGEDTTIQETVKVNTRMQCFSVTERGSFYPGSGFAFYSLDYMRTPLDVGTESTW
EVEVVAHIRPAADTGVLFALWAPDLRAVPLSVALVDYHSTKKLKKQLVVLAVEHT
ALALMEIKVCDGQEHVVTVSLRDGEATLEVDGTRGQSEVSAAQLQERLAVLERHLR
SPVLTFAGGLPDVPVTSAPVTAFYRGCMTLEVNRRLLDLDEAAYKHSDITAHSCPPV
EPAAA (Gas6-Gla EGF deleted)
DYKDHDGDYKDHDIDYKDDDDK* (FLAG)
2 αAβ[Fab]-Gas6 (Aducanumab(Fab)-Gas6-FLAG, nucleotide
sequence) SEQ ID NO: 157
ATGGAGACAGACACACTCCTGCTATGGGTACTGCTGCTCTGGGTTCCAGGTTCCA
CTGGTGAC (SS)
GAGGTTCAGCTTGTCGAGTCTGGGGGGGGAGTCGTTCAGCCAGGTAGAAGCCTC
AGACTGAGCTGTGCCGCAAGTGGGTTTGCTTTTTCATCTTACGGTATGCACTGGG
TGAGACAGGCTCCTGGCAAAGGACTCGAGTGGGTCGCTGTAATATGGTTCGATG
GTACAAAGAAATACTATACCGATAGTGTGAAAGGAAGATTCACCATTTCACGAG
ACAACAGTAAAAATACCTTGTACCTTCAGATGAACACCCTGAGAGCAGAAGACA
CAGCCGTGTACTACTGCGCCAGAGATAGAGGTATCGGAGCAAGGCGTGGTCCCT
ATTATATGGATGTGTGGGGGAAGGGAACAACAGTGACTGTGAGCTCTGCCTCCA
CCAAGGGCCCATCGGTCTTCCCCCTGGCACCCTCCTCCAAGAGCACCTCTGGGGG
CACAGCGGCCCTGGGCTGCCTGGTCAAGGACTACTTCCCCGAACCGGTGACGGT
GTCGTGGAACTCAGGCGCCCTGACCAGCGGCGTGCACACCTTCCCGGCTGTCCT
ACAGTCCTCAGGACTCTACTCCCTCAGCAGCGTGGTGACTGTGCCCTCTAGCAGC
TTGGGCACCCAGACCTACATCTGCAACGTGAATCACAAGCCCAGCAACACCAAG
GTGGACAAGAAAGTTGAGCCCAAATCTTGTGACAAAACTCAC (Adu-VH-CH1)
GGCGGAGGTGGAAGCGGAGGCGGTGGAAGC (Linker)
GACATCTTGCCGTGCGTGCCCTTCAGCGTGGCCAAGAGTGTGAAGTCCTTGTACC
TGGGCCGGATGTTCAGTGGGACCCCCGTGATCCGACTGCGCTTCAAGAGGCTGC
AGCCCACCAGGCTGGTAGCTGAGTTTGACTTCCGGACCTTTGACCCCGAGGGCA
TCCTCCTCTTTGCCGGAGGCCACCAGGACAGCACCTGGATCGTGCTGGCCCTGAG
AGCCGGCCGGCTGGAGCTGCAGCTGCGCTACAACGGTGTCGGCCGTGTCACCAG
CAGCGGCCCGGTCATCAACCATGGCATGTGGCAGACAATCTCTGTTGAGGAGCT
GGCGCGGAATCTGGTCATCAAGGTCAACAGGGATGCTGTCATGAAAATCGCGGT
GGCCGGGGACTTGTTCCAACCGGAGCGAGGACTGTATCATCTGAACCTCACCGT
GGGAGGTATTCCCTTCCATGAGAAGGACCTCGTGCAGCCTATAAACCCTCGTCTG
GATGGCTGTATGAGGAGCTGGAACTGGCTGAACGGAGAAGACACCACCATCCA
GGAAACGGTGAAAGTGAACACGAGGATGCAGTGCTTCTCGGTGACGGAGAGAG
GCTCTTTCTACCCCGGGAGCGGCTTCGCCTTCTACAGCCTGGACTACATGCGGAC
CCCTCTGGACGTCGGGACTGAATCAACCTGGGAAGTAGAAGTCGTGGCTCACAT
CCGCCCAGCCGCAGACACAGGCGTGCTGTTTGCGCTCTGGGCCCCCGACCTCCGT
GCCGTGCCTCTCTCTGTGGCACTGGTAGACTATCACTCCACGAAGAAACTCAAG
AAGCAGCTGGTGGTCCTGGCCGTGGAGCATACGGCCTTGGCCCTAATGGAGATC
AAGGTCTGCGACGGCCAAGAGCACGTGGTCACCGTCTCGCTGAGGGACGGTGAG
GCCACCCTGGAGGTGGACGGCACCAGGGGCCAGAGCGAGGTGAGCGCCGCGCA
GCTGCAGGAGAGGCTGGCCGTGCTCGAGAGGCACCTGCGGAGCCCCGTGCTCAC
CTTTGCCGGCGGCCTGCCAGATGTGCCGGTGACTTCAGCGCCAGTCACCGCGTTC
TACCGCGGCTGCATGACACTGGAGGTCAACCGGAGGCTGCTGGACCTGGACGAG
GCGGCGTACAAGCACAGCGACATCACGGCCCACTCCTGCCCCCCCGTGGAGCCC
GCCGCAGCC (Gas6-Gla EGF deleted)
GACTACAAAGACCATGACGGTGATTATAAAGATCATGACATCGACTACAAGGAT
GACGATGACAAGtga (FLAG)

TABLE 9
1. αAβ[Mab]-Gas6 (Aducanumab heavy chain (Mab)-Gas6-FLAG,
amino acid sequence) SEQ ID NO: 158
METDTLLLWVLLLWVPGSTGD (SS)
EVQLVESGGGVVQPGRSLRLSCAASGFAFSSYGMHWVRQAPGKGLEWVAVIWFDG
TKKYYTDSVKGRFTISRDNSKNTLYLQMNTLRAEDTAVYYCARDRGIGARRGPYY
MDVWGKGTTVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLS
LSPGK (Adu-Heavy chain)
GGGGSGGGGS (Linker)
DILPCVPFSVAKSVKSLYLGRMFSGTPVIRLRFKRLQPTRLVAEFDFRTFDPEGILLFA
GGHQDSTWIVLALRAGRLELQLRYNGVGRVTSSGPVINHGMWQTISVEELARNLVI
KVNRDAVMKIAVAGDLFQPERGLYHLNLTVGGIPFHEKDLVQPINPRLDGCMRSWN
WLNGEDTTIQETVKVNTRMQCFSVTERGSFYPGSGFAFYSLDYMRTPLDVGTESTW
EVEVVAHIRPAADTGVLFALWAPDLRAVPLSVALVDYHSTKKLKKQLVVLAVEHT
ALALMEIKVCDGQEHVVTVSLRDGEATLEVDGTRGQSEVSAAQLQERLAVLERHLR
SPVLTFAGGLPDVPVTSAPVTAFYRGCMTLEVNRRLLDLDEAAYKHSDITAHSCPPV
EPAAA (Gas6-Gla EGF deleted)
DYKDHDGDYKDHDIDYKDDDDK* (FLAG)
2. αAβ[Mab]-Gas6 (Aducanumab(Mab)-Gas6-FLAG, nucleotide
sequence) SEQ ID NO: 159
ATGGAGACAGACACACTCCTGCTATGGGTACTGCTGCTCTGGGTTCCAGGTTCCA
CTGGTGAC (SS)
GAGGTTCAGCTTGTCGAGTCTGGGGGGGGAGTCGTTCAGCCAGGTAGAAGCCTC
AGACTGAGCTGTGCCGCAAGTGGGTTTGCTTTTTCATCTTACGGTATGCACTGGG
TGAGACAGGCTCCTGGCAAAGGACTCGAGTGGGTCGCTGTAATATGGTTCGATG
GTACAAAGAAATACTATACCGATAGTGTGAAAGGAAGATTCACCATTTCACGAG
ACAACAGTAAAAATACCTTGTACCTTCAGATGAACACCCTGAGAGCAGAAGACA
CAGCCGTGTACTACTGCGCCAGAGATAGAGGTATCGGAGCAAGGCGTGGTCCCT
ATTATATGGATGTGTGGGGGAAGGGAACAACAGTGACTGTGAGCTCTGCCTCCA
CCAAGGGCCCATCGGTCTTCCCCCTGGCACCCTCCTCCAAGAGCACCTCTGGGGG
CACAGCGGCCCTGGGCTGCCTGGTCAAGGACTACTTCCCCGAACCGGTGACGGT
GTCGTGGAACTCAGGCGCCCTGACCAGCGGCGTGCACACCTTCCCGGCTGTCCT
ACAGTCCTCAGGACTCTACTCCCTCAGCAGCGTGGTGACTGTGCCCTCTAGCAGC
TTGGGCACCCAGACCTACATCTGCAACGTGAATCACAAGCCCAGCAACACCAAG
GTGGACAAGAAAGTTGAGCCCAAATCTTGTGACAAAACTCACACATGCCCACCG
TGCCCAGCACCTGAACTCCTGGGGGGACCGTCAGTCTTCCTCTTCCCCCCAAAAC
CCAAGGACACCCTCATGATCTCCCGGACCCCTGAGGTCACATGCGTGGTGGTGG
ACGTGAGCCACGAAGACCCTGAGGTCAAGTTCAACTGGTACGTGGACGGCGTGG
AGGTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAGGAG
TACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCC
CGGGATGAGCTGACCAAGAACCAGGTCAGCCTGACCTGCCTGGTCAAAGGCTTC
TATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTACAGC
AAGCTCACCGTGGACAAGAGCAGGTGGCAGCAGGGGAACGTCTTCTCATGCTCC
GTGATGCATGAGGCTCTGCACAACCACTACACGCAGAAGAGCCTCTCCCTGTCC
CCGGGTAAA (Adu-Heavy chain)
GGCGGAGGTGGAAGCGGAGGCGGTGGAAGC (Linker)
GACATCTTGCCGTGCGTGCCCTTCAGCGTGGCCAAGAGTGTGAAGTCCTTGTACC
TGGGCCGGATGTTCAGTGGGACCCCCGTGATCCGACTGCGCTTCAAGAGGCTGC
AGCCCACCAGGCTGGTAGCTGAGTTTGACTTCCGGACCTTTGACCCCGAGGGCA
TCCTCCTCTTTGCCGGAGGCCACCAGGACAGCACCTGGATCGTGCTGGCCCTGAG
AGCCGGCCGGCTGGAGCTGCAGCTGCGCTACAACGGTGTCGGCCGTGTCACCAG
CAGCGGCCCGGTCATCAACCATGGCATGTGGCAGACAATCTCTGTTGAGGAGCT
GGCGCGGAATCTGGTCATCAAGGTCAACAGGGATGCTGTCATGAAAATCGCGGT
GGCCGGGGACTTGTTCCAACCGGAGCGAGGACTGTATCATCTGAACCTCACCGT
GGGAGGTATTCCCTTCCATGAGAAGGACCTCGTGCAGCCTATAAACCCTCGTCTG
GATGGCTGTATGAGGAGCTGGAACTGGCTGAACGGAGAAGACACCACCATCCA
GGAAACGGTGAAAGTGAACACGAGGATGCAGTGCTTCTCGGTGACGGAGAGAG
GCTCTTTCTACCCCGGGAGCGGCTTCGCCTTCTACAGCCTGGACTACATGCGGAC
CCCTCTGGACGTCGGGACTGAATCAACCTGGGAAGTAGAAGTCGTGGCTCACAT
CCGCCCAGCCGCAGACACAGGCGTGCTGTTTGCGCTCTGGGCCCCCGACCTCCGT
GCCGTGCCTCTCTCTGTGGCACTGGTAGACTATCACTCCACGAAGAAACTCAAG
AAGCAGCTGGTGGTCCTGGCCGTGGAGCATACGGCCTTGGCCCTAATGGAGATC
AAGGTCTGCGACGGCCAAGAGCACGTGGTCACCGTCTCGCTGAGGGACGGTGAG
GCCACCCTGGAGGTGGACGGCACCAGGGGCCAGAGCGAGGTGAGCGCCGCGCA
GCTGCAGGAGAGGCTGGCCGTGCTCGAGAGGCACCTGCGGAGCCCCGTGCTCAC
CTTTGCCGGCGGCCTGCCAGATGTGCCGGTGACTTCAGCGCCAGTCACCGCGTTC
TACCGCGGCTGCATGACACTGGAGGTCAACCGGAGGCTGCTGGACCTGGACGAG
GCGGCGTACAAGCACAGCGACATCACGGCCCACTCCTGCCCCCCCGTGGAGCCC
GCCGCAGCC (Gas6-Gla EGF deleted)
GACTACAAAGACCATGACGGTGATTATAAAGATCATGACATCGACTACAAGGAT
GACGATGACAAG (FLAG)
TGA

### Preparation Examples 6-8

As non-limiting exemplary embodiment of the binding molecule containing a scaffold protein between the first region and the second region, wherein the Gas6 and anti-amyloid antibody scFv (in this example, aducanumab scFv) are employed as the first region and the second region, respectively, a single chain Fc region with reduced or abolished Fc receptor binding affinity are manufactured. The sequences employed in the construction are shown in Table 10.

As another non-limiting exemplary embodiment of the binding molecule containing a scaffold protein between the first region and the second region, wherein the Gas6 and anti-amyloid antibody scFv (in this example, aducanumab scFv) are employed as the first region and the second region, respectively, a heterodimeric binding molecule is manufactured. The first polypeptide of the heterodimeric binding molecule comprises anti-amyloid antibody scFv, Fc region (DD), and Gas6, and the second polypeptide of the heterodimeric binding molecule comprises anti-amyloid antibody scFv region and Fc region (KK). Still another non-limiting exemplary embodiment of the binding molecule containing a scaffold protein between the first region and the second region, a homodimer comprising two polypeptides which each comprise anti-amyloid antibody scFv (as second region), Fc region (scaffold), and Gas6 is manufactured. The peptide sequences are shown in Table 10

TABLE 10
Preparation Example 6: Single polypeptide fusion molecule:
anti-amyloid antibody-MFc-Gas6-His (SEQ ID NO: 162)
METDTLLLWVLLLWVPGSTGD (SS)
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPLTFGGGTKVEIKGGGGSGGGG
SGGGGSEVQLVESGGGVVQPGRSLRLSCAASGFAFSSYGMHWVRQAPGKGLEWVA
VIWFDGTKKYYTDSVKGRFTISRDNSKNTLYLQMNTLRAEDTAVYYCARDRGIGAR
RGPYYMDVWGKGTTVTVSS (anti-amyloid antibody-scFv)
GGGGSGGGGS (linker)
APEFLGGPSVFLFPPKPKDTLYITREPEVTCVVVDVSQEDPEVQFNWYVDGVEVHNA
KTKPREEQFNSTYRVVSVLTVLHQDWLNGKEYKCKVSNKGLPSSIEKTISKAKGQPR
EPQVYTFPPEQEEMTKNQVSLRCLVKGFYPSDIAVEWESNGQPENN
YKTTKPVLDSDGSFRLESRLTVDKSRWQEGNVESCSVMHEACSWHLCKSLSLSLGK
(Monomeric Fc with reduced or abolished Fc gamma receptor
binding affinity)
GGGGSGGGGSGGGGS (linker)
DILPCVPFSVAKSVKSLYLGRMESGTPVIRLRFKRLQPTRLVAEFDFRTFDPEGILLFA
GGHQDSTWIVLALRAGRLELQLRYNGVGRVTSSGPVINHGMWQTISVEELARNLVI
KVNRDAVMKIAVAGDLFQPERGLYHLNLTVGGIPFHEKDLVQPINPRLDGCMRSWN
WLNGEDTTIQETVKVNTRMQCFSVTERGSFYPGSGFAFYSLDYMRTPLDVGTESTW
EVEVVAHIRPAADTGVLFALWAPDLRAVPLSVALVDYHSTKKLKKQLVVLAVEHT
ALALMEIKVCDGQEHVVTVSLRDGEATLEVDGTRGQSEVSAAQLQERLAVLERHLR
SPVLTFAGGLPDVPVTSAPVTAFYRGCMTLEVNRRLLDLDEAAYKHSDITAHSCPPV
EPAAA (Gas6)
HHHHHH (His)
Preparation Example 7: Heterodimeric fusion molecule
comprising monovalent first region and monovalent second
region: First polypeptide comprising anti-amyloid
antibody-Fc(DD)-Gas6-His (SEQ ID NO: 163)
METDTLLLWVLLLWVPGSTGD (SS)
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPLTFGGGTKVEIKGGGGSGGGG
SGGGGSEVQLVESGGGVVQPGRSLRLSCAASGFAFSSYGMHWVRQAPGKGLEWVA
VIWFDGTKKYYTDSVKGRFTISRDNSKNTLYLQMNTLRAEDTAVYYCARDRGIGAR
RGPYYMDVWGKGTTVTVSS (anti-amyloid antibody-scFv)
GGGGSGGGGS (linker)
DKTHTCPPCPAPELLGGPSVFLFPPKPKDTLYITREPEVTCVVVDVSHEDPEVKFNW
YVDGVEVHNAKTKPREEQYASTYRVVSVLTVLHQDWLNGKEYKCKVSNKALPAPI
EKTISKAKGQPREPQVYTLPPSREEMTKNQVSLTCLVKGFYPSDIAVEWESNGQPEN
NYDTTPPVLDSDGSFFLYSDLTVDKSRWQQGNVFSCSVMHEALHNHYTQKSLSLSP
GK (Fc(DD))
GGGGSGGGGSGGGGS (linker)
DILPCVPFSVAKSVKSLYLGRMFSGTPVIRLRFKRLQPTRLVAEFDFRTFDPEGILLFA
GGHQDSTWIVLALRAGRLELQLRYNGVGRVTSSGPVINHGMWQTISVEELARNLVI
KVNRDAVMKIAVAGDLFQPERGLYHLNLTVGGIPFHEKDLVQPINPRLDGCMRSWN
WLNGEDTTIQETVKVNTRMQCFSVTERGSFYPGSGFAFYSLDYMRTPLDVGTESTW
EVEVVAHIRPAADTGVLFALWAPDLRAVPLSVALVDYHSTKKLKKQLVVLAVEHT
ALALMEIKVCDGQEHVVTVSLRDGEATLEVDGTRGQSEVSAAQLQERLAVLERHLR
SPVLTFAGGLPDVPVTSAPVTAFYRGCMTLEVNRRLLDLDEAAYKHSDITAHSCPPV
EPAAA (Gas6)
HHHHHH (His)
Second polypeptide comprising anti-amyloid antibody-
Fc(KK)-Gas6-His (SEQ ID NO: 164)
METDTLLLWVLLLWVPGSTGD (SS)
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPLTFGGGTKVEIKGGGGSGGGG
SGGGGSEVQLVESGGGVVQPGRSLRLSCAASGFAFSSYGMHWVRQAPGKGLEWVA
VIWFDGTKKYYTDSVKGRFTISRDNSKNTLYLQMNTLRAEDTAVYYCARDRGIGAR
RGPYYMDVWGKGTTVTVSS (anti-amyloid antibody-scFv)
GGGGSGGGGS (linker)
DKTHTCPPCPAPELLGGPSVFLFPPKPKDTLYITREPEVTCVVVDVSHEDPEVKFNW
YVDGVEVHNAKTKPREEQYASTYRVVSVLTVLHQDWLNGKEYKCKVSNKALPAPI
EKTISKAKGQPREPQVYTLPPSRKEMTKNQVSLTCLVKGFYPSDIAVEWESNGQPEN
NYKTTPPVLKSDGSFFLYSKLTVDKSRWQQGNVFSCSVMHEALHNHYTQKSLSLSP
GK (Fc(KK))
Preparation Example 8: Single polypeptide fusion molecule
comprising monovalent first region, scaffold, and monovalent
second region: Anti-amyloid antibody-Fc-Gas6-His
(SEQ ID NO: 165)
METDTLLLWVLLLWVPGSTGD (SS)
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPLTFGGGTKVEIKGGGGSGGGG
SGGGGSEVQLVESGGGVVQPGRSLRLSCAASGFAFSSYGMHWVRQAPGKGLEWVA
VIWFDGTKKYYTDSVKGRFTISRDNSKNTLYLQMNTLRAEDTAVYYCARDRGIGAR
RGPYYMDVWGKGTTVTVSS (anti-amyloid antibody-scFv)
GGGGSGGGGS (linker)
DKTHTCPPCPAPELLGGPSVFLFPPKPKDTLYITREPEVTCVVVDVSHEDPEVKFNW
YVDGVEVHNAKTKPREEQYASTYRVVSVLTVLHQDWLNGKEYKCKVSNKALPAPI
EKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSP
GK (Fc region with reduced or abolished Fc gamma receptor
binding affinity)
GGGGSGGGGSGGGGS (linker)
DILPCVPFSVAKSVKSLYLGRMFSGTPVIRLRFKRLQPTRLVAEFDFRTFDPEGILLFA
GGHQDSTWIVLALRAGRLELQLRYNGVGRVTSSGPVINHGMWQTISVEELARNLVI
KVNRDAVMKIAVAGDLFQPERGLYHLNLTVGGIPFHEKDLVQPINPRLDGCMRSWN
WLNGEDTTIQETVKVNTRMQCFSVTERGSFYPGSGFAFYSLDYMRTPLDVGTESTW
EVEVVAHIRPAADTGVLFALWAPDLRAVPLSVALVDYHSTKKLKKQLVVLAVEHT
ALALMEIKVCDGQEHVVTVSLRDGEATLEVDGTRGQSEVSAAQLQERLAVLERHLR
SPVLTFAGGLPDVPVTSAPVTAFYRGCMTLEVNRRLLDLDEAAYKHSDITAHSCPPV
EPAAA (Gas6)
HHHHHH (His)

### Preparation Example 9

As non-limiting exemplary embodiment of the binding molecule containing a scaffold protein between the first region and the second region, a bispecific antibody, wherein a scFv of anti-Axl antibody and anti-amyloid antibody are employed as the first region and the second region, respectively, is manufactured. See, FIG. 1J. A heavy chain of the bispecific antibody has the following sequence of Table 11 and the light chain of anti-amyloid antibody light chain has the sequence of SEQ ID NO: 161. The Fc region of the heavy chain contains NA mutation to reduced or abolish Fc gamma receptor binding affinity.

TABLE 11
Bispecific Antibody fusion molecule: Anti-amyloid
Ab-anti-Axl ScFv (first polypeptide)
(SEQ ID NO: 166)
METDTLLLWVLLLWVPGSTGD (SS)
EVQLVESGGGVVQPGRSLRLSCAASGFAFSSYGMHWVRQAPGKGLEWVA
VIWFDGTKKYYTDSVKGRFTISRDNSKNTLYLQMNTLRAEDTAVYYCAR
DRGIGARRGPYYMDVWGKGTTVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNA
KTKPREEQYASTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHN
HYTQKSLS
LSPGK (anti-amyloid-antibody heavy chain)
GGGGSGGGGS (Linker)
EVKLVESGGDLVKPGGSLKLSCAASGFTFSSYGMSWVRQTPDKRLEWVA
TISSGGSYTYYPDSVKGRFTISRDNAKNTLYLQMSSLKSEDTAMYYCAR
HPIYYTYDDTMDYWGQGTSVTVSSGGGGSGGGGSGGGGSDIVLTQSPAI
MAASPGEKVTMTCSASSSVSSGNFHWYQQKPGTSPKLWIYRTSNLASGV
PARFSGSGSGTSYSLTISSMEAEDAATYYCQQWSGYPWTFGGGTKLEIK
(anti-Axl scFv)

### Preparation Example 10

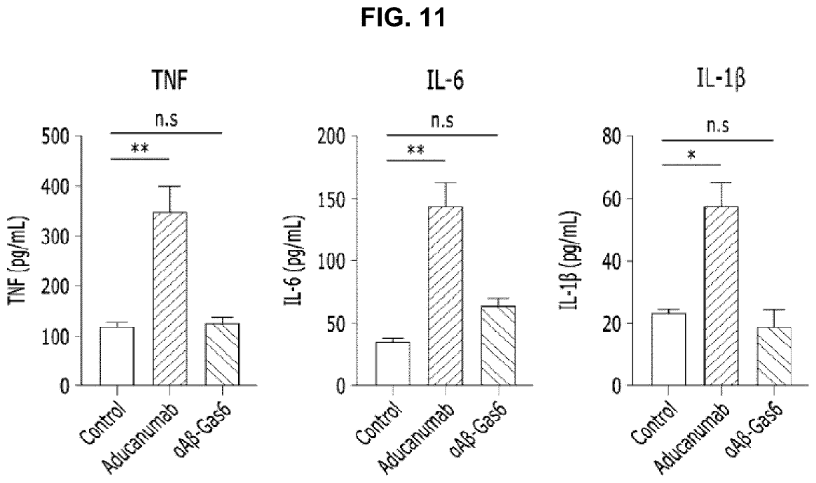

As non-limiting exemplary embodiment of the binding molecule containing a scaffold protein between the first region and the second region, a homodimeric bispecific antibody, wherein a scFv of anti-Axl antibody and a scFv region of an anti-amyloid antibody are employed as the first region and the second region, respectively, is manufactured. See FIG. 11. The bispecific antibody comprises a first polypeptide and a second polypeptide, which are identical to each other and each comprises the sequence of SEQ ID NO: 167. The structure of the first/second polypeptide is illustrated in Table 12, and the Fc region scaffold contains N-A mutation to reduced or abolish Fc gamma receptor binding affinity.

TABLE 12
Bispecific homodimeric comprising two polypeptides:
anti-amyloid Ab-anti-Axl ScFv (SEQ ID NO: 167)
METDTLLLWVLLLWVPGSTGD (SS)
EVQLVESGGGVVQPGRSLRLSCAASGFAFSSYGMHWVRQAPGKGLEWVAVI
WFDGTKKYYTDSVKGRFTISRDNSKNTLYLQMNTLRAEDTAVYYCARDRGI
GARRGPYYMDVWGKGTTVTVSSASTKGPSVFPLAPSSKSTSGGTAALGCLV
KDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
(anti-amyloid-antibody heavy chain)
GGGGSGGGGS (Linker)
EVKLVESGGDLVKPGGSLKLSCAASGFTFSSYGMSWVRQTPDKRLEWVATI
SSGGSYTYYPDSVKGRFTISRDNAKNTLYLQMSSLKSEDTAMYYCARHPIY
YTYDDTMDYWGQGTSVTVSSGGGGSGGGGSGGGGSDIVLTQSPAIMAASPG
EKVTMTCSASSSVSSGNFHWYQQKPGTSPKLWIYRTSNLASGVPARFSGSG
SGTSYSLTISSMEAEDAATYYCQQWSGYPWTFGGGTKLEIK (anti-Axl
scFv)

### Preparation Example 11

As another non-limiting exemplary embodiment of the binding molecule containing a scaffold protein between the first region and the second region, a heterodimeric bispecific antibody, wherein a scFv of anti-Axl antibody and an anti-amyloid antibody are employed as the first region and the second region, respectively, is manufactured. The first polypeptide of a heavy chain of the bispecific antibody has the following sequence of SEQ ID NO: 168 of Table 13 below, the second polypeptide of a heavy chain of the bispecific antibody comprises the sequence of SEQ ID NO: 169 of Table 13 below, and the light chain of anti-amyloid antibody has the sequence of SEQ ID NO: 161. The Fe region contains NA mutation to reduce or abolish Fc gamma receptor binding affinity and the polypeptides of the Fc region form a hetero dimer (DD-KK).

TABLE 13
Bispecific heterodimeric comprising bivalent second
region and bivalent first region: first polypeptide
of heavy chain of anti-amyloid Ab-anti-Axl ScFv
(with Fc region DD) (SEQ ID NO: 168)
METDTLLLWVLLLWVPGSTGD (SS)
EVQLVESGGGVVQPGRSLRLSCAASGFAFSSYGMHWVRQAPGKGLEWVAVI
WFDGTKKYYTDSVKGRFTISRDNSKNTLYLQMNTLRAEDTAVYYCARDRGI
GARRGPYYMDVWGKGTTVTVSSASTKGPSVFPLAPSSKSTSGGTAALGCLV
KDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVYT
LPPSRDELTKNQVSLTCLVKGFYPSDIAVEWESNGQPENNYDTTPPVLDSD
GSFFLYSDLTVDKSRWQQGNVFSCSVMHEALHNHYTQKSLSLSPGK
(anti-amyloid antibody heavy chain (DD))
GGGGSGGGGS (Linker)
EVKLVESGGDLVKPGGSLKLSCAASGFTFSSYGMSWVRQTPDKRLEWVATI
SSGGSYTYYPDSVKGRFTISRDNAKNTLYLQMSSLKSEDTAMYYCARHPIY
YTYDDTMDYWGQGTSVTVSSGGGGSGGGGSGGGGSDIVLTQSPAIMAASPG
EKVTMTCSASSSVSSGNFHWYQQKPGTSPKLWIYRTSNLASGVPARFSGSG
SGTSYSLTISSMEAEDAATYYCQQWSGYPWTFGGGTKLEIK (anti-Axl
scFv)
Second polypeptide of heavy chain of anti-amyloid
Ab (with Fc region KK) (SEQ ID NO: 169)
METDTLLLWVLLLWVPGSTGD (SS)
EVQLVESGGGVVQPGRSLRLSCAASGFAFSSYGMHWVRQAPGKGLEWVAVI
WFDGTKKYYTDSVKGRFTISRDNSKNTLYLQMNTLRAEDTAVYYCARDRGI
GARRGPYYMDVWGKGTTVTVSSASTKGPSVFPLAPSSKSTSGGTAALGCLV
KDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVYT
LPPSRKELTKNQVSLTCLVKGFYPSDIAVEWESNGQPENNYKTTPPVLKSD
GSFFLYSKLTVDKSRWQQGNVFSCSVMHEALHNHYTQKSLSLSPGK
(anti-amyloid antibody heavy chain (KK))

### Experimental Example 1. Gas6-Based Fusion Molecule Targeting Beta-Amyloid (I): Beta-Amyloid Binding Domain in scFv Form

### 1-1. Analysis of Expression of Fusion Molecule in Transfected Cells

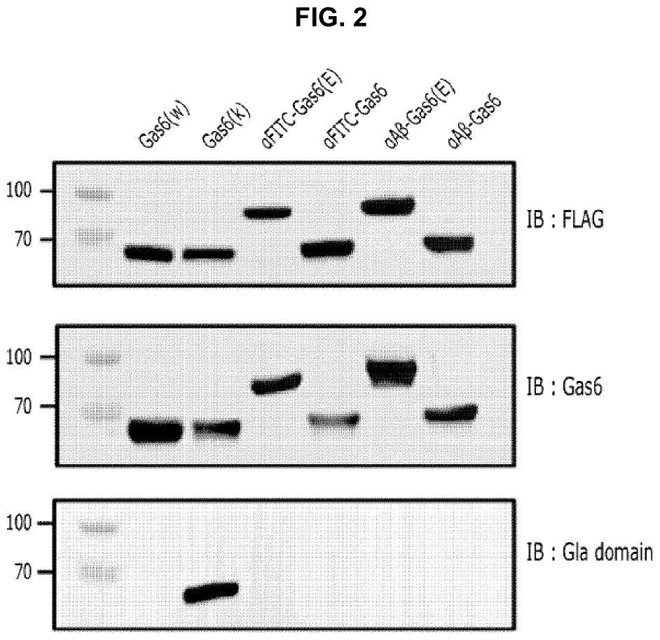

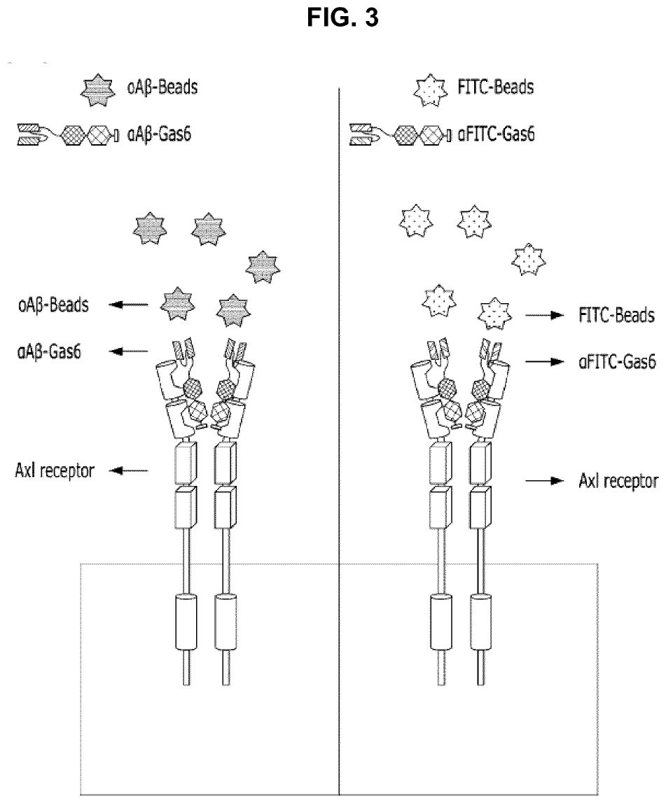

After plasmid transfection into HEK293 cells, the expression of the fusion molecule containing the Flag tag according to Preparation Example 1 analyzed by Western blot analysis using the Flag tag, and the results are shown in FIG. 2.

### 1-2. Analysis of Beta-Amyloid Specific Binding Affinity of Prepared Fusion Molecules

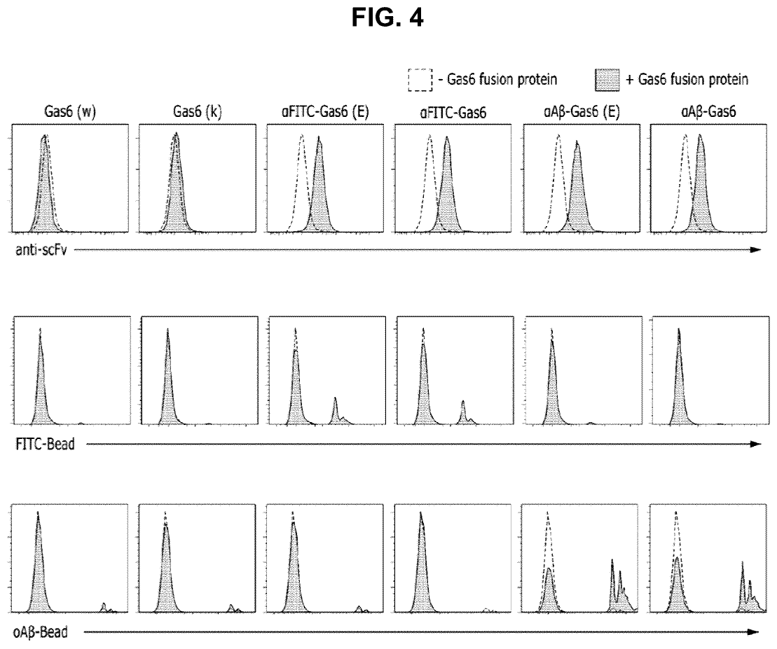

To verify whether each of αAβ-Gas6(E), αAβ-Gas6, αFITC-Gas6(E), and αFITC-Gas6 can selectively recognize beta-amyloid and FITC, the culture broth secreted from the HEK293 transfected with each plasmid was collected and subjected to an experiment using beta-amyloid oligomer and FITC-conjugated beads. The results showed that αAβ-Gas6 (E) and αAβ-Gas6 recognized only beta-amyloid oligomer beads, and αFITC-Gas6 (E) and αFITC-Gas6 recognized only FITC beads, thereby inducing phagocytosis, as shown in FIG. 4.

Although αAβ-Gas6 (E) and αAβ-Gas6 were shown to exhibit similar activities, it was found that αAβ-Gas6 obtained by additionally removing the EGF domain of Gas6 could be obtained in high yield without aggregation in the protein purification process. Thus, αAβ-Gas6 was used in subsequent experiments.

### 1-3. Analysis of Mechanism of Action of Prepared Fusion Molecule

### (1) Analysis Using Cell Line

An in vitro Aβ engulfment assay was developed, in which beta-amyloid oligomers are conjugated with a pH indicator and hence can emit red fluorescence in intracellular lysosomes when they are uptaken by phagocytosis.

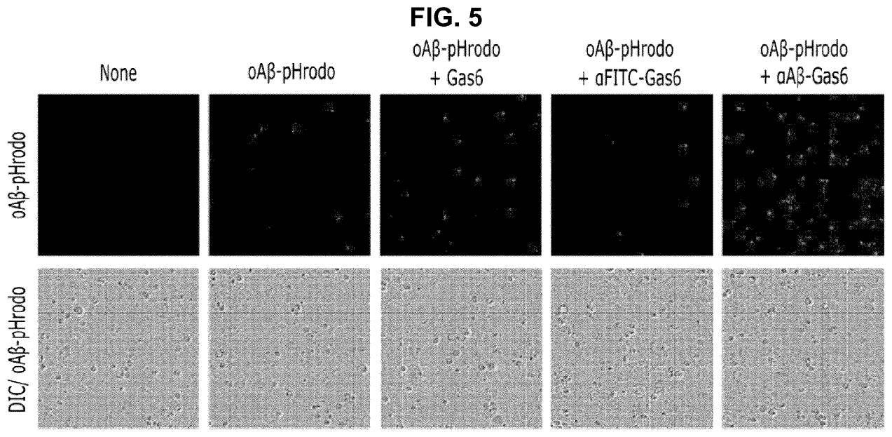

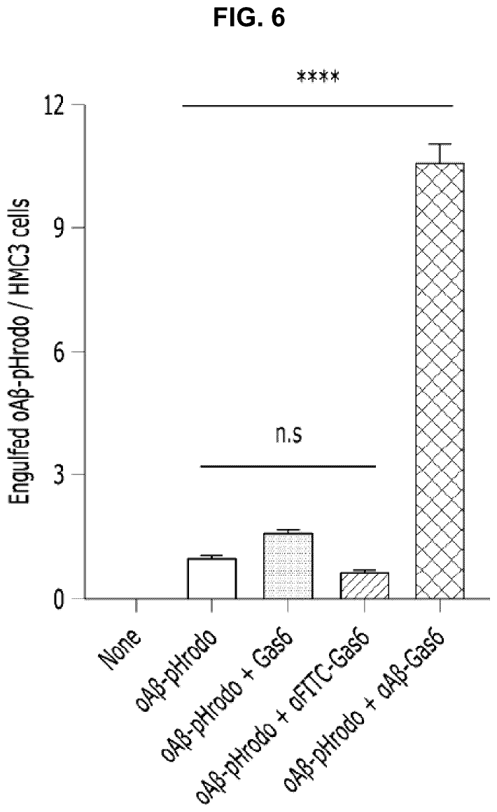

As a result of performing the in vitro Aβ engulfment assay with HMC3 cells, a human microglial cell line expressing TAM receptors, it was shown that beta-amyloid oligomers were selectively cleared by αAβ-Gas6 (FIGS. 5 and 6).

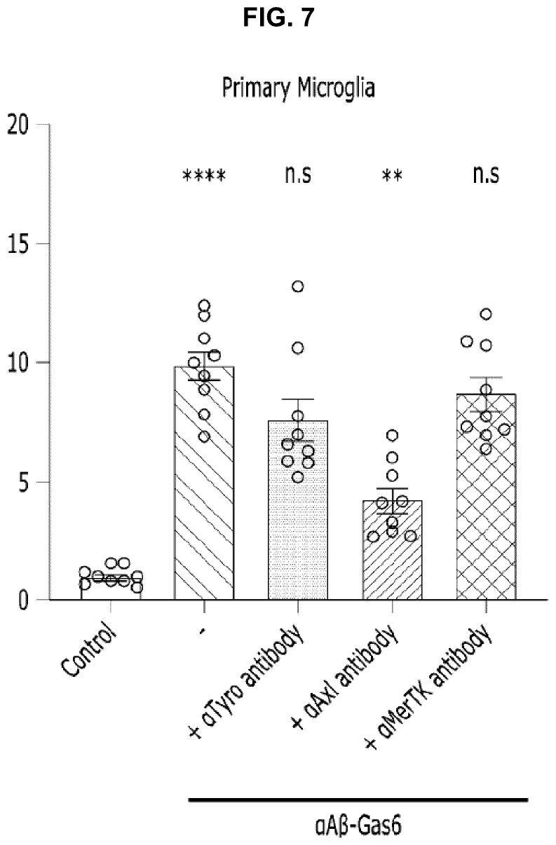

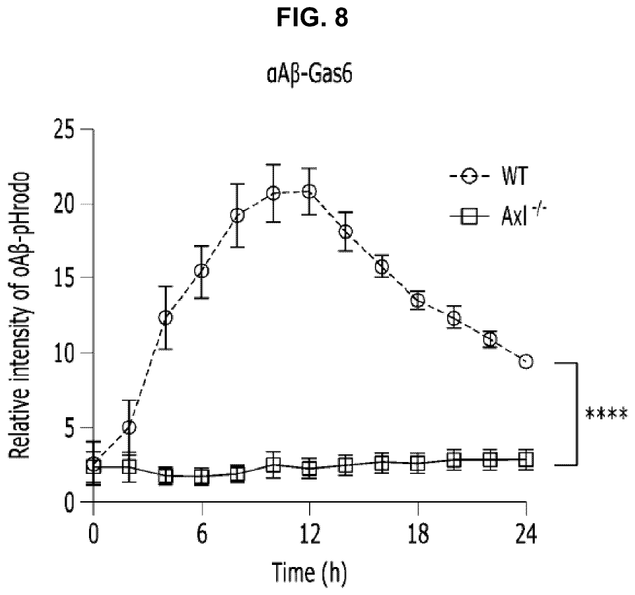

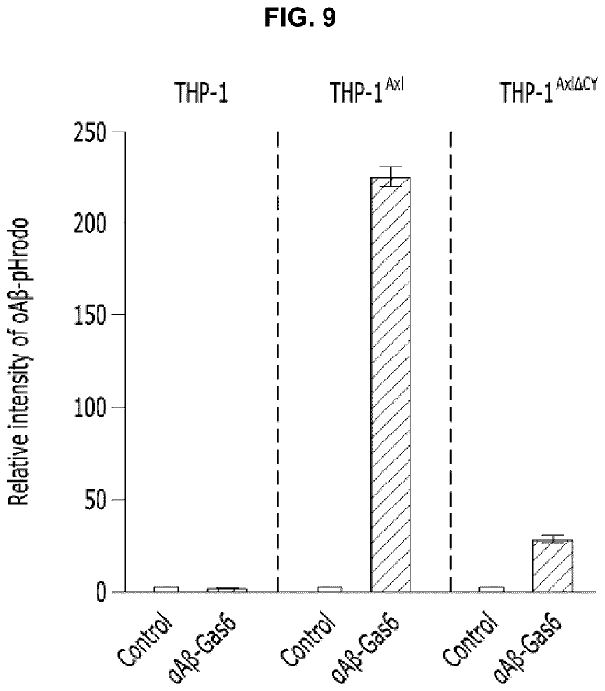

In particular, in an experiment where cells were treated additionally with an antibody that interferes with the function of TAM receptors, it was confirmed that αAβ-Gas6 cleared beta-amyloid oligomers mainly through Axl among Tyro3, MerTK, and Axl (FIGS. 7 to 9). In fact, when Axl was removed from HMC3 cells, the activity of αAβ-Gas6 significantly decreased. In addition, THP-1, which is a human monocyte cell line that does not express TAM receptors, did not show an increase in beta-amyloid clearance by αAβ-Gas6, while THP-Axl cells overexpressing Axl exhibited a significantly increased ability to clear beta-amyloid fibrils in a manner dependent on αAβ-Gas6.

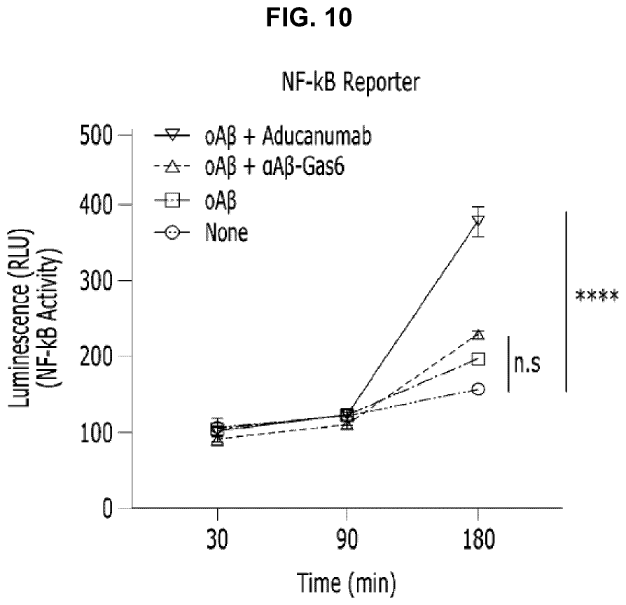

Next, since THP-Axl cells express both Axl and Fc receptors, the degree of inflammatory response induced upon beta-amyloid uptake by each of αAβ-Gas6 and aducanumab was analyzed in those cells. To this end, the NF-kB reporter was first expressed in THP-Axl cells, and each of a control, αAβ-Gas6 and aducanumab was added to the cells together with beta-amyloid oligomers. As a result, it was confirmed that, when aducanumab was added, the expression of the NF-kB reporter significantly increased, but when αAβ-Gas6 was added, the NF-kB reporter was expressed at or below the control level (FIG. 10). In addition, as a result of measuring the secreted protein levels of IL-1b, IL-6 and TNF, which are the three most representative inflammatory cytokines, it was shown that, when THP-Axl cells were treated with aducanumab, the protein levels of the inflammatory cytokines in the treated cells significantly increased compared to those in the control group (FIG. 11). In contrast, importantly, the levels of these inflammatory cytokines in the cells treated with αAβ-Gas6 did not increase compared to those in the control group. This is a key result that, as our hypothesis suggests, the αAβ-Gas6 fusion phagocytosis-inducing protein does not induce an inflammatory response when phagocytosing a target substance through a TAM receptor, which is similar to recognition and efferocytosis of naturally apoptotic cells.

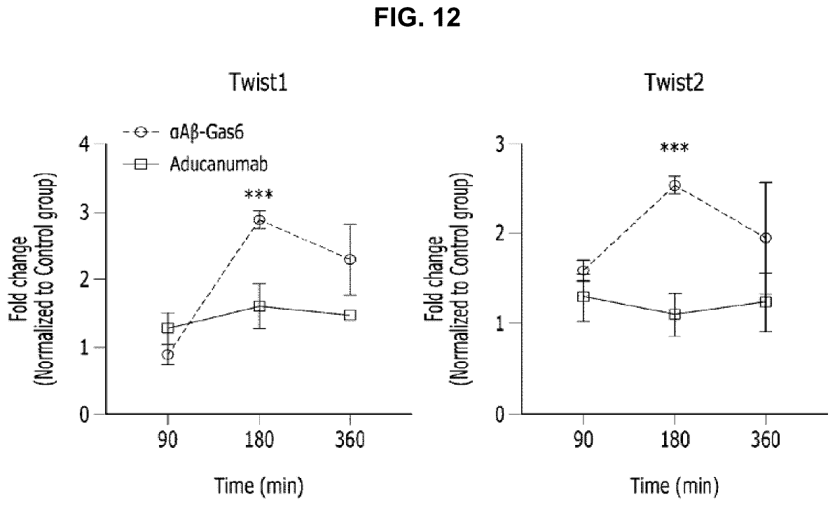

In addition, unlike aducanumab, αAβ-Gas6 increased the expression of Twist1/2 gene, which is known as a mechanism of suppressing inflammatory responses (FIG. 12).

### (2) Analysis Using Astrocytes and Microglia

To examine whether astrocytes and microglia, which are cells expressing TAM receptors in the brain, can clear beta-amyloid through αAβ-Gas6, primary astrocytes and microglia obtained from mouse brains were separately purified and then cultured. Then, each of purified αAβ-Gas6 and aducanumab was added to the cells together with beta-amyloid fibrils, and the degree of clearance of beta-amyloid fibrils was measured in real time.

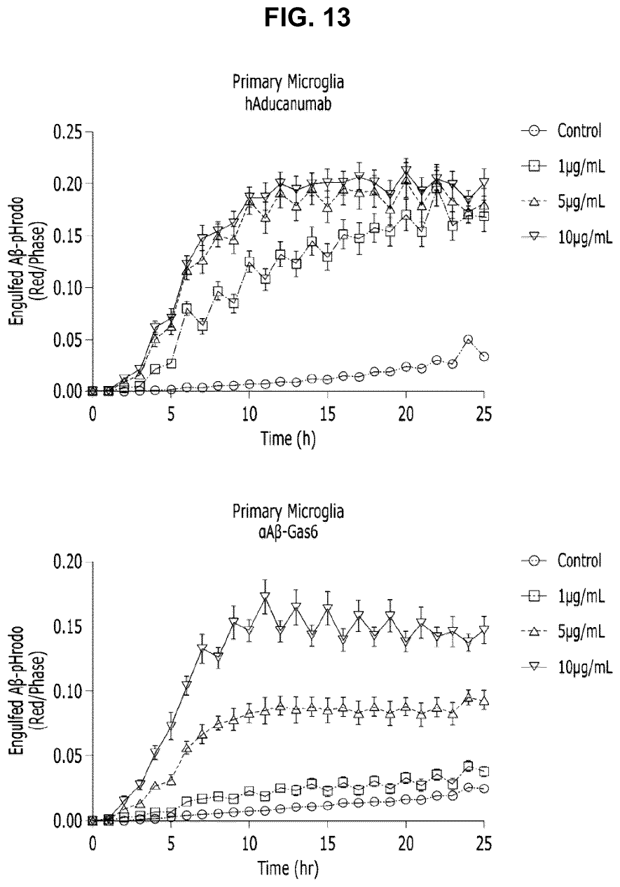

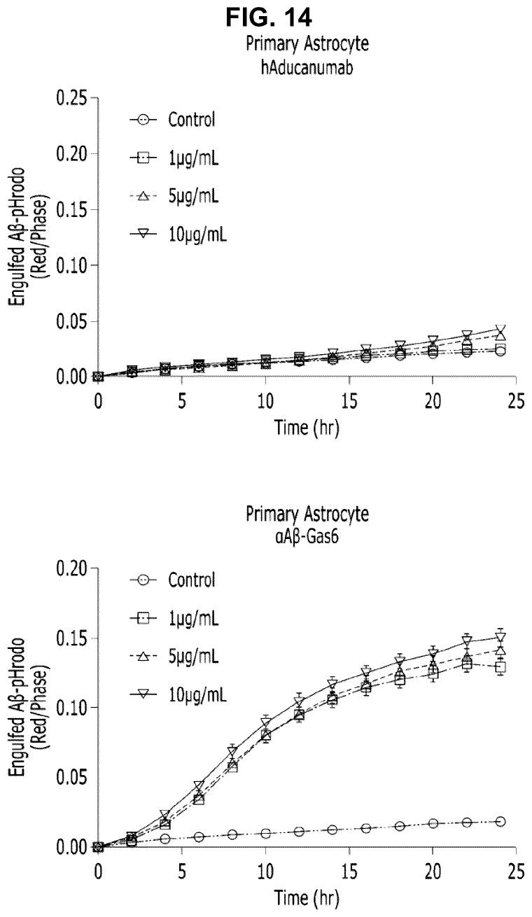

The results showed that αAβ-Gas6 increased the beta-amyloid clearing ability of microglia in a concentration-dependent manner, which is similar to the results obtained in HMC3 which is a cell line expressing Axl (FIG. 13). Importantly, it was shown that, when aducanumab was added, the beta-amyloid clearing ability of astrocytes did not change at all, but when αAβ-Gas6 was added, the beta-amyloid clearing ability of astrocytes significantly increased in a concentration-dependent manner (FIG. 14). This suggests that αAβ-Gas6 significantly enhances the beta-amyloid clearing ability of astrocytes, which was previously insignificant, because astrocytes do not express Fc receptors but express TAM receptors.

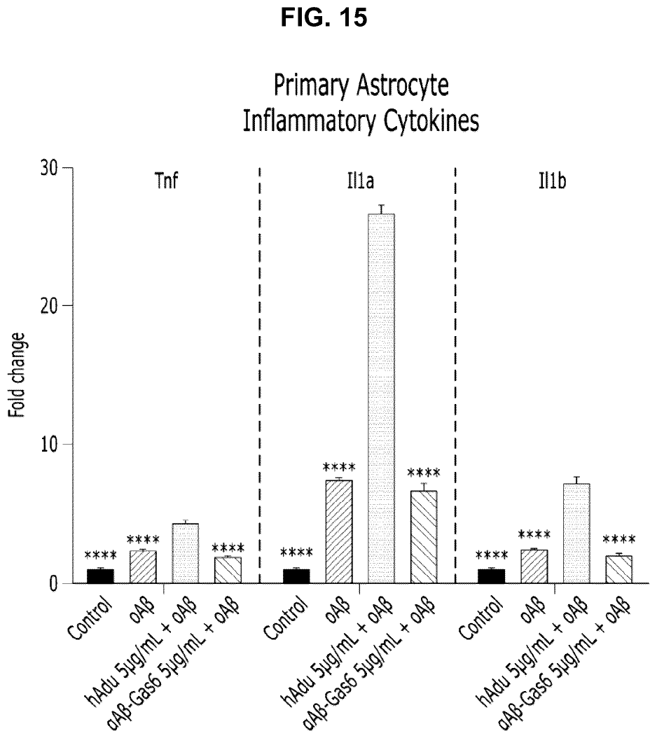

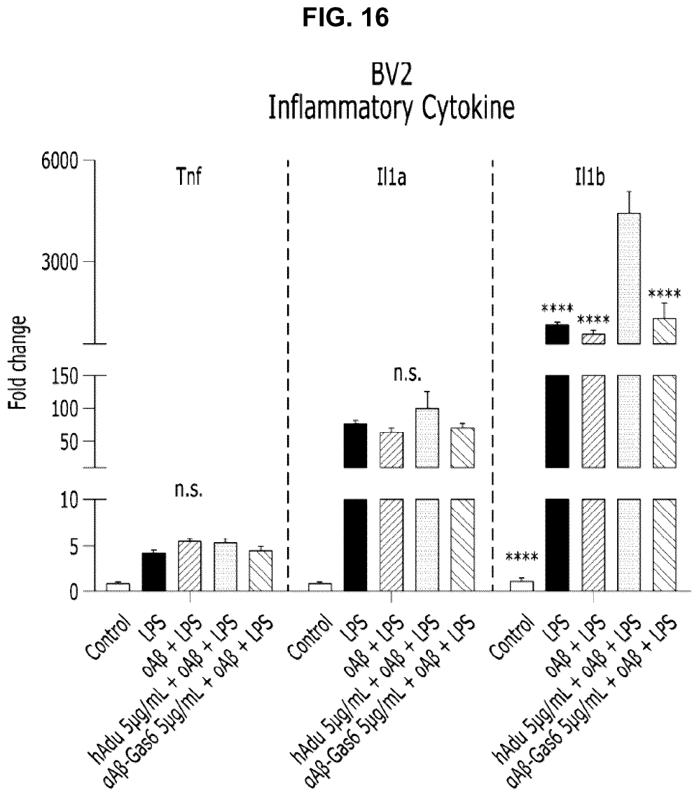

Each of αAβ-Gas6 and aducanumab was added to astrocytes and the microglia cell line BV2 together with beta-amyloid fibrils to increase beta-amyloid uptake, and then the mRNA levels of TNF, IL-1a and IL-1b in each cell line were measured to determine the degree of inflammatory responses (FIGS. 15 and 16). As a result, similar to the results obtained in the cell lines, it was shown that, when the cells were treated with aducanumab, the levels of transcripts and proteins of the above inflammatory cytokines in the astrocytes and BV2 cells significantly increased compared to those in the control group, but when the cells were treated with αAβ-Gas6, the levels of these inflammatory cytokines in the cells did not increase compared to those in the control group.

As described above, it has been found that the use of the αAβ-Gas6 fusion phagocytosis inducer may be a groundbreaking method of effectively clearing beta-amyloid plaques accumulated in the patient's brain, through astrocytes and microglia without causing an inflammatory response, which is a serious side effect of existing monoclonal antibody therapeutics. This could be a very encouraging result that can significantly improve current treatment strategies.

### 1-4. Evaluation of In Vivo Efficacy

### (1) Efficacy According to Introduction of Fusion Molecule or Expression Vector Containing the Same

5XFAD mice were used as Alzheimer's disease model mice. Since 5XFAD mice simultaneously express 5 genes with mutations, the onset at which beta-amyloid plaques are generated in the mice is early, and pathological symptoms caused by beta-amyloid plaques can be studied from 3 to 4 months of age regardless of aging.

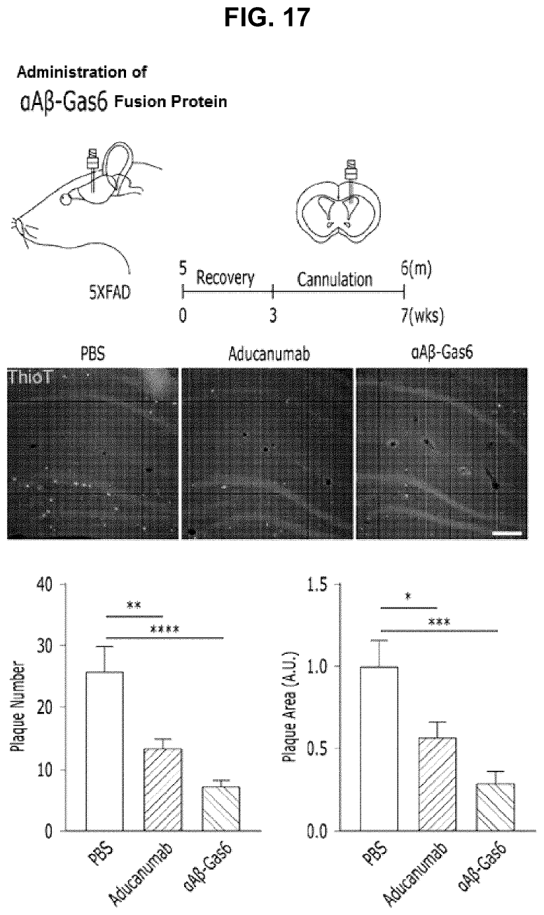

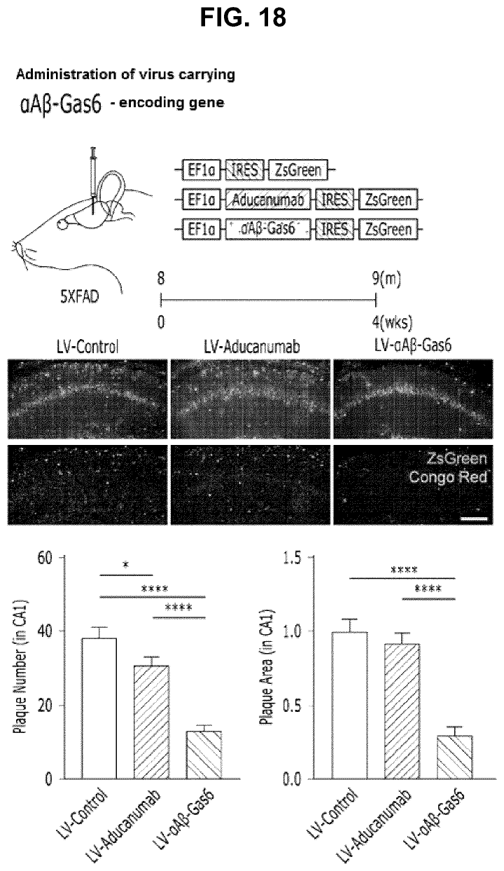

To verify the effect of αAβ-Gas6 in vivo through the 5XFAD model, αAβ-Gas6 was delivered to the brain in two different ways. Through previous studies, it is known that aducanumab is not delivered well to the brain by intravascular injection or intraperitoneal injection even in Alzheimer's disease model mice. Thus, to accurately compare and analyze the effects of αAβ-Gas6 with aducanumab, 1) direct cannulation was performed in the mouse brain, and each of purified αAβ-Gas6 and aducanumab was injected once a day into the ventricle of the brain for 3 weeks, and 2) each of αAβ-Gas6 and aducanumab was made in lentiviral form to be expressed in the hippocampus of the mouse through stereotaxic injection. Importantly, it was found that the number of beta-amyloid plaques significantly decreased both when the purified αAβ-Gas6 protein was added and when the gene was expressed in lentiviral form (FIGS. 17 and 18).

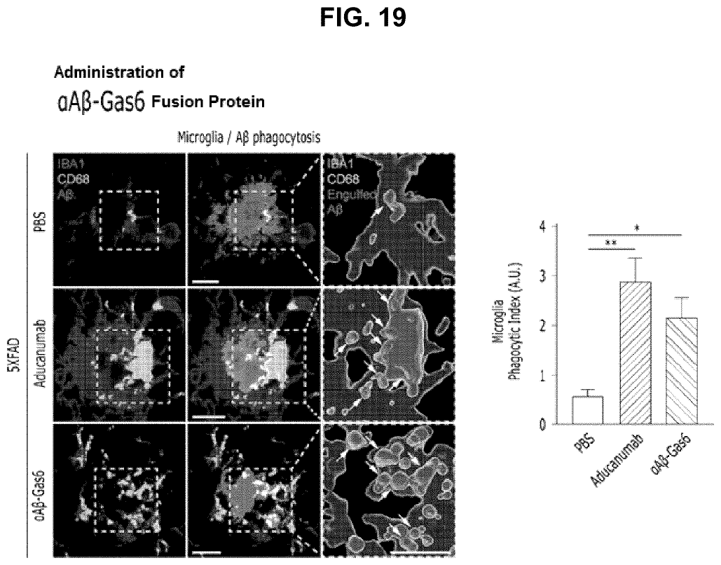

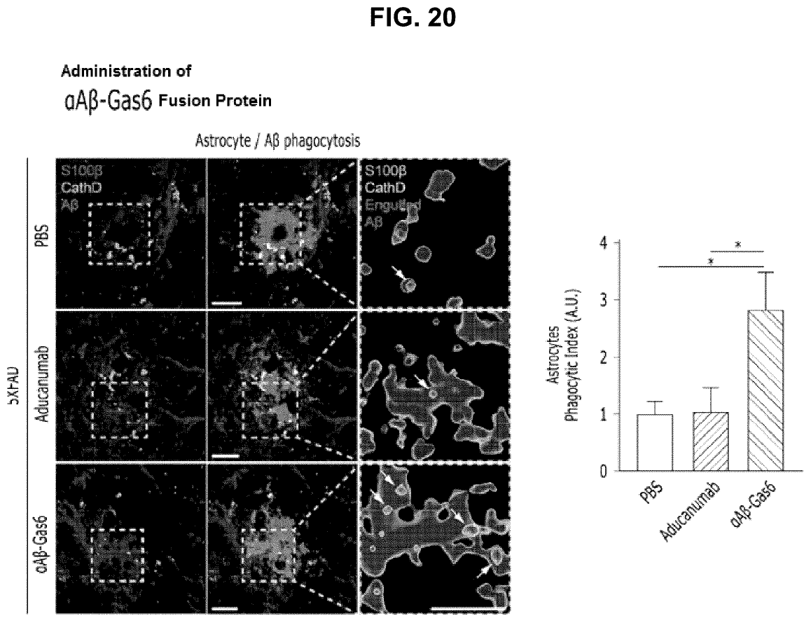

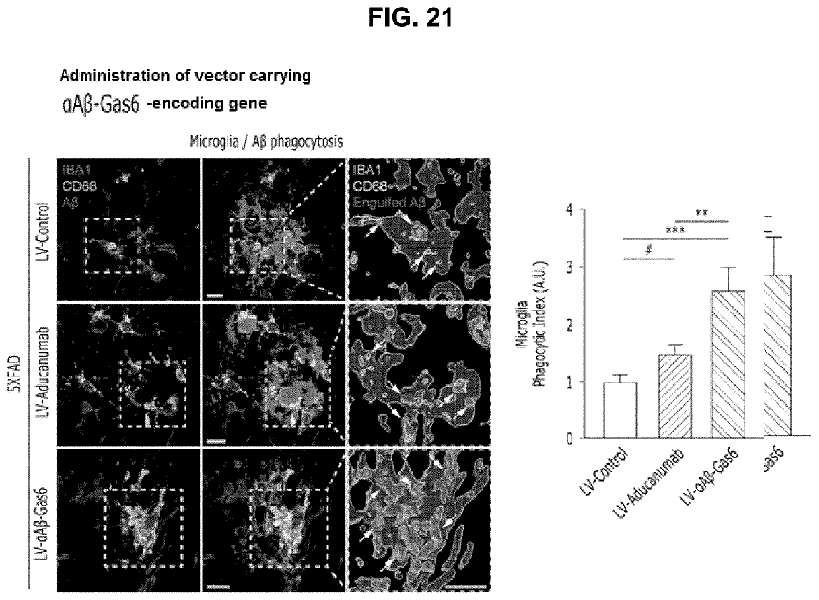

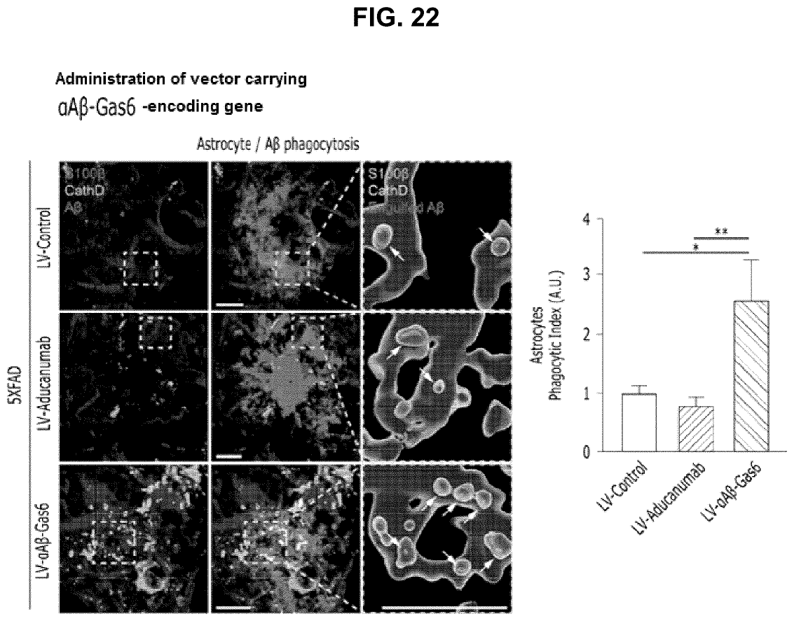

In addition, by quantifying the levels of beta-amyloid in lysosomes of microglia and astrocytes after αAβ-Gas6 was delivered to the brain in the form of protein or virus, it was shown that the ability to clear beta-amyloid significantly increased in both types of cells (FIGS. 19 to 22).

This suggests that, since TAM receptors are expressed in both microglia and astrocytes, microglia and astrocytes can recognize and clear beta-amyloid when αAβ-Gas6 is introduced therein, which is similar to the results of the in vitro studies.

### (2) Comparison of Effects of Antibody Therapeutics and Fusion Molecule of the Present Invention

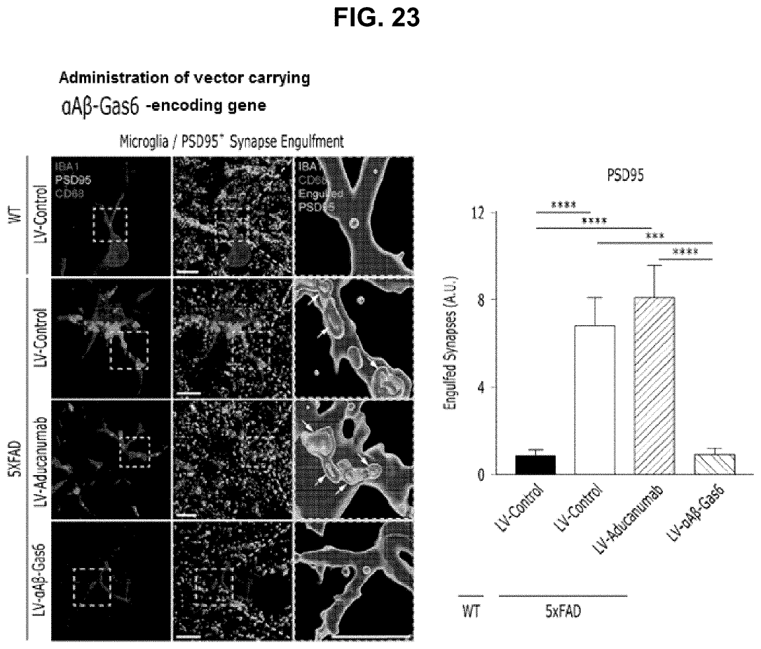

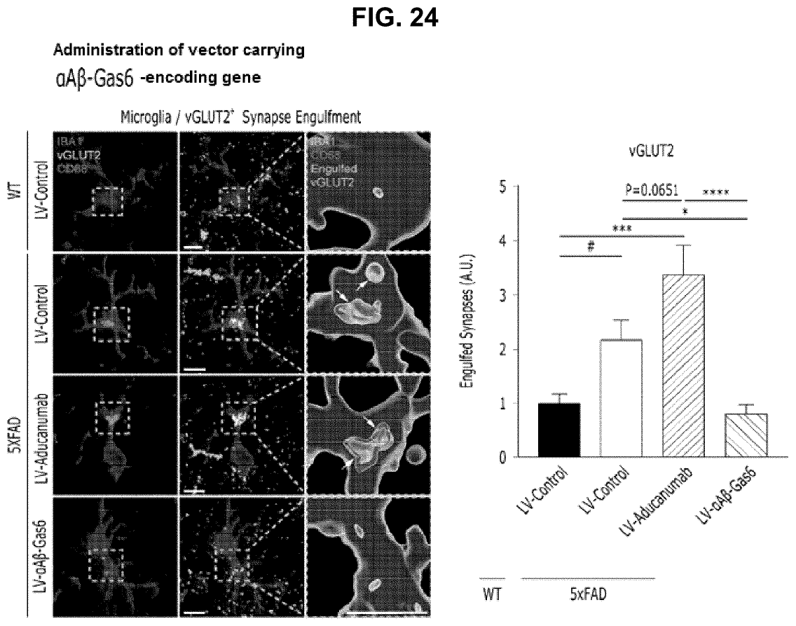

It is known that, in Alzheimer's disease, synapses are indiscriminately removed by microglia, resulting in a decrease in the number of synapses. Surprisingly, this phenomenon was aggravated when aducanumab was delivered to Alzheimer's model mice, but when αAβ-Gas6 was expressed in a viral form, abnormal removal of synapses by microglia was restored to a normal level (FIGS. 23 and 24).

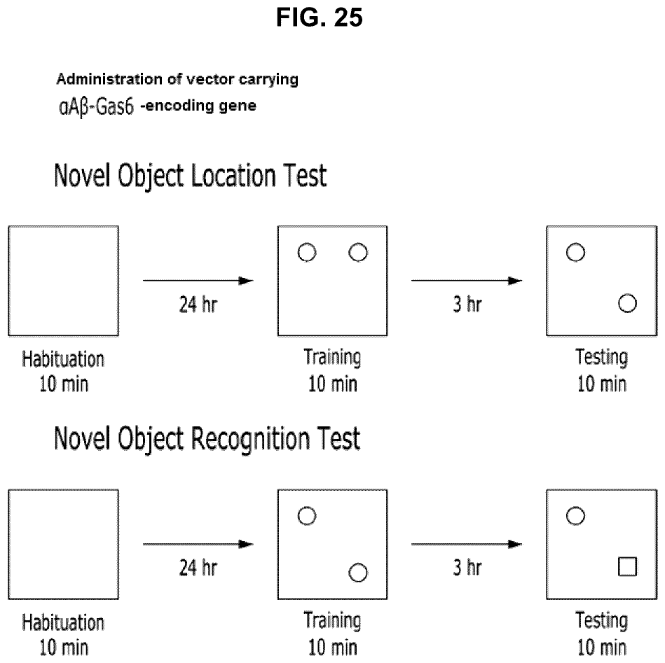

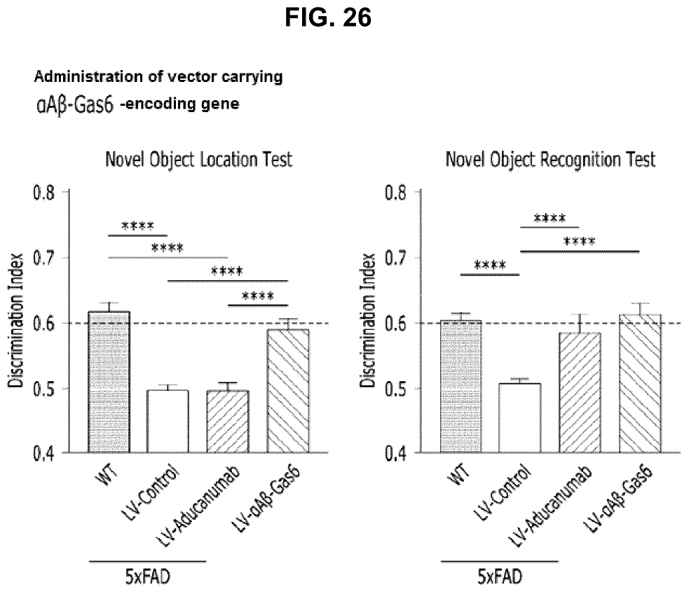

In addition, as in the results from a cognitive and memory test for remembering the shape or location of a new object in Alzheimer's model mice according to the protocol shown in FIG. 25, it was confirmed that the expression of αAβ-Gas6 exhibited significantly superior cognitive and memory recovery effects compared to aducanumab (FIG. 26).

In addition, to verify whether the chimeric phagocytic protein of the present disclosure is effective in clearing various target substances, phagocytosis-inducing proteins specific for tau and alpha-synuclein (αSyn) in addition to beta-amyloid were prepared as described in Preparation Examples 2 and 3, and the target substance clearing effects thereof were tested following protocols in Experimental Examples 2 and 3.

### 1-5. Assay of Fusion Molecules of Preparation Examples 6-11

By following the procedure of Example 1-1 through 1-4, the properties and in vivo efficacy of clearing amyloids are evaluated for the fusion molecules of Preparation Examples 6-11.

### Experimental Example 2. Gas6-Based Fusion Molecule Targeting Tau

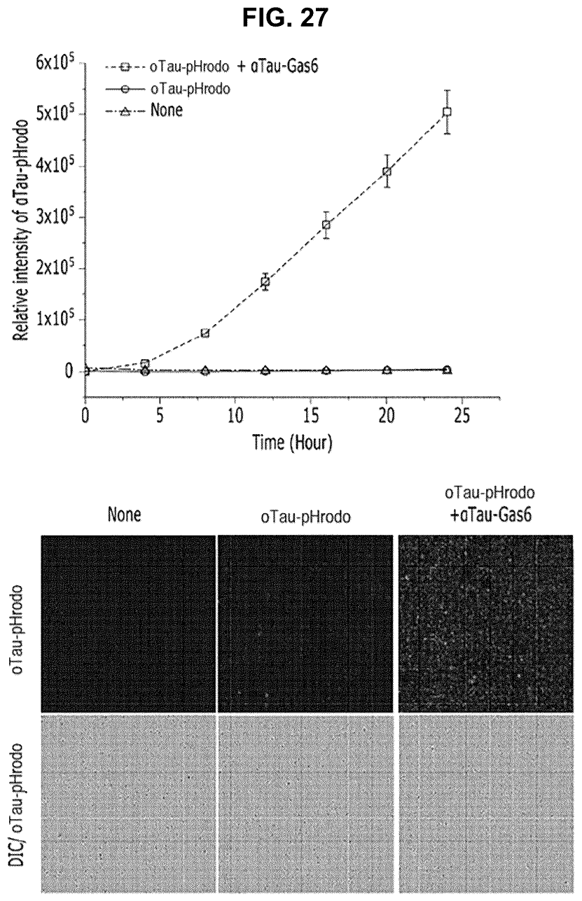

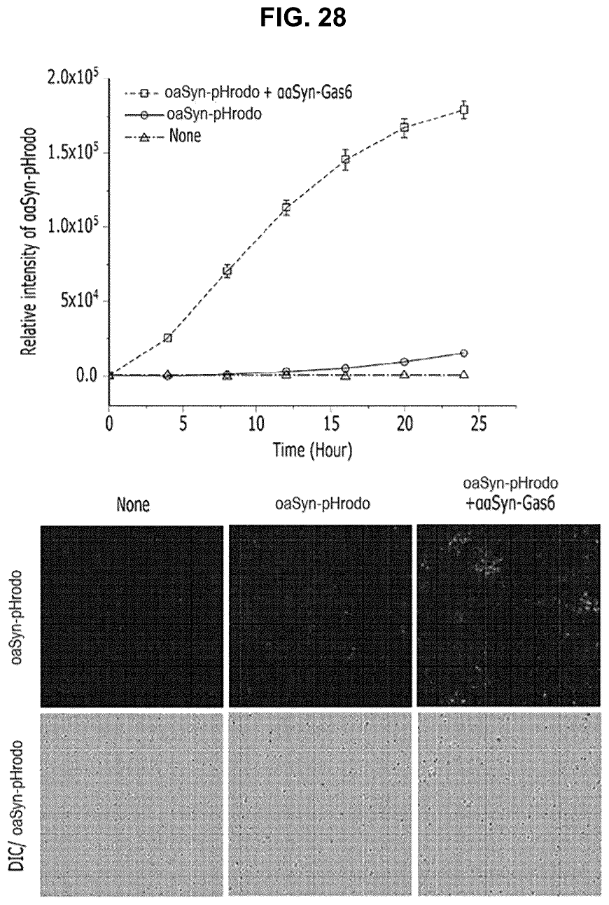

An in vitro tau engulfment assay was developed, in which tau oligomers are conjugated with a pH indicator and hence can emit red fluorescence in intracellular lysosomes when they are uptaken by phagocytosis. HMC3 cells, a human microglial cell line expressing TAM receptors, were treated with a culture medium expressing the phagocytosis-inducing protein [αTau-Gas6]according to Preparation Example 2, and in vitro tau engulfment assay was performed. As the result shown in FIG. 27, it was confirmed that tau oligomers were selectively cleared by αTau-Gas6.

### Experimental Example 3. Gas6-Based Fusion Molecule Targeting Alpha-Synuclein

An in vitro αSyn engulfment assay was developed, in which alpha-synuclein (αSyn) oligomers are conjugated with a pH indicator and hence can emit red fluorescence in intracellular lysosomes when they are uptaken by phagocytosis. HMC3 cells, a human microglial cell line expressing TAM receptors, were treated with a culture medium expressing the phagocytosis-inducing protein [ααSyn-Gas6]according to Preparation Example 3, and in vitro tau engulfment assay was performed. As the result shown in FIG. 27, it was confirmed that αSyn oligomers were selectively cleared by ααSyn-Gas6.

### Experimental Example 4. ProS1-Based Fusion Molecule Targeting Beta-Amyloid

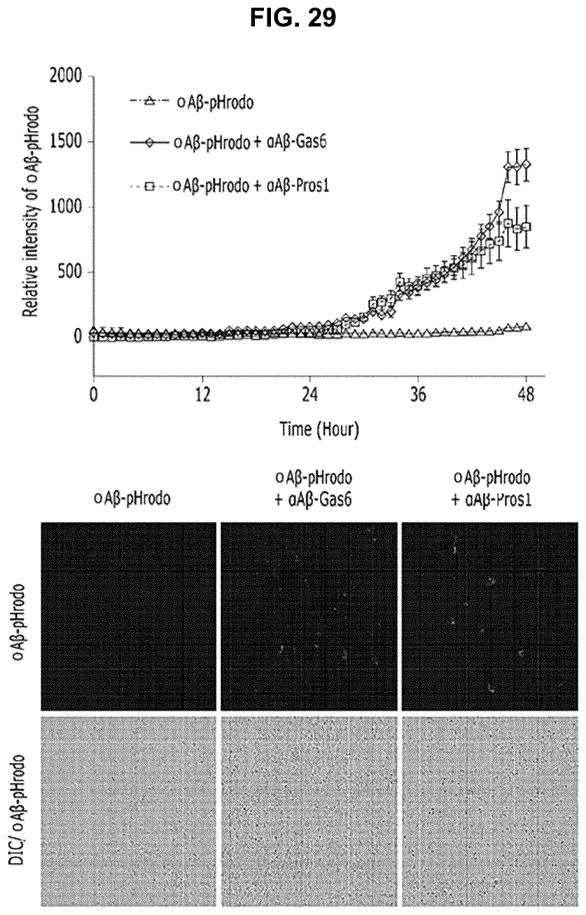

Next, to verify whether the chimeric phagocytosis-inducing protein prepared using a ligand for TAM receptor other than Gas6 is also effective, αAβ-ProS1 was prepared as described in Preparation Example 4 using the ProS1 ligand, and the efficacy thereof was evaluated. To this end, primary-cultured mouse astrocytes expressing TAM receptors were treated with a culture medium expressing αAβ-ProS1, and the in vitro Aβ engulfment assay used in Experimental Example 1-3 was performed. As the result shown in FIG. 29, it was confirmed that beta-amyloid oligomers were selectively cleared by αAβ-ProS1.

### Experimental Example 5. Gas6-Based Fusion Molecule Targeting Beta-Amyloid (II): Beta-Amyloid Binding Regions in the Forms of Fab and Mab

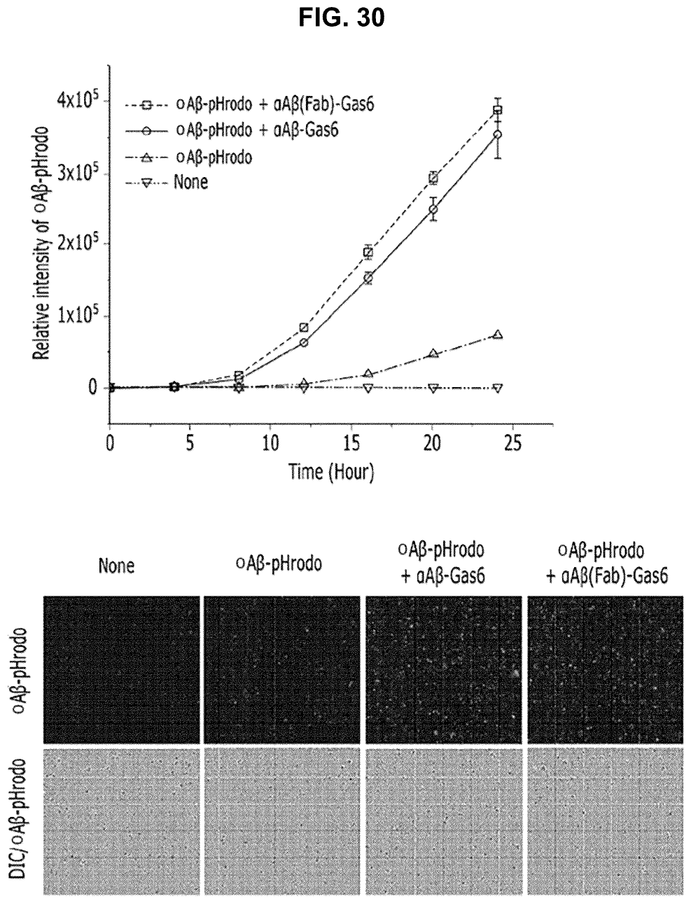

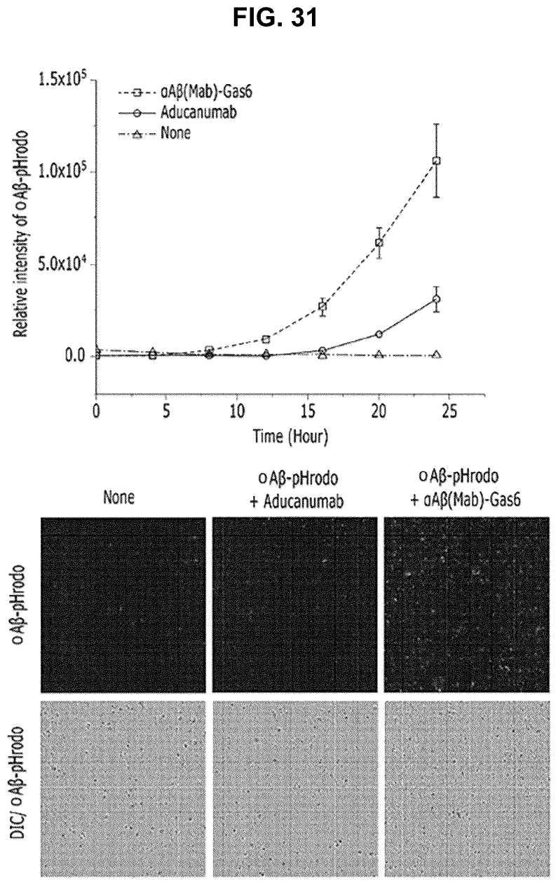

Next, to verify whether various target-binding regions other than scFv may be used as target protein-binding domains in the preparation of chimeric phagocytosis-inducing proteins, phagocytosis-inducing proteins were prepared according to Preparation Example 5 using an antigen-binding fragment (Fab) or a complete-form monoclonal antibody (Mab) instead of an scFv and were subjected to an experiment (αAβ[Fab]-Gas6 and αAβ[Mab]-Gas6). To this end, HMC3 cells, a human microglial cell line expressing TAM receptors, were treated with a culture medium expressing each of αAβ[Fab]-Gas6 and αAβ[Mab]-Gas6, and the in vitro Aβ engulfment assay used in Experimental Example 1-3 was performed. As the results shown in FIGS. 30 and 31, it was confirmed that beta-amyloid oligomers were selectively cleaved by each of αAβ[Fab]-Gas6 and αAβ[Mab]-Gas6.

The scope of the present disclosure is defined by the appended claims, and all changes or modifications derived from the meaning and scope of the claims and equivalents thereto should be construed as being included in the scope of the present invention.

The fusion molecules having phagocytosis-inducing activity according to the embodiment of the present disclosure can solve the problem of tissue damage caused by activation of an inflammatory response, which occurs in the prior art. Accordingly, the fusion molecules could effectively clear abnormally accumulated substances such as beta-amyloid, tau, alpha-synuclein, huntingtin or prion protein, and thus may be used to prevent or treat diseases caused by these abnormally accumulated substances, for example, Alzheimer's disease, Parkinson's disease, Huntington's disease, or prion disease. Therefore, it may be used in the therapeutics industry for treatment of the above diseases.

All publications, patent applications, patents, and other references mentioned herein are expressly incorporated herein by reference in their entireties.

1-23. (canceled)

24. A binding molecule comprising a first region capable of binding to a TAM receptor and a second region capable of specifically binding to a target substance, said target substance being a substance of which aberrant accumulation in a living tissue is characteristic of or associated with a disease, wherein the first region and the second region are coupled to each other directly or via a linker,
wherein the first region comprises
(a) a TAM receptor ligand;
(b) an anti-Axl antibody or an antigen-binding fragment thereof;
(c) an anti-Tyro3 antibody or an antigen-binding fragment thereof; or
(d) an anti-MerTK antibody or an antigen-binding fragment thereof, with proviso that when the first region comprises an anti-MerTK antibody or an antigen-binding fragment thereof, the molecule is not a bispecific antibody; or
(e) combinations thereof,
wherein the TAM receptor ligand is selected from the group consisting of ProS1, Tubby, Tulp1, Gal3, and combinations thereof, or an Axl-binding fragment thereof.

25. The binding molecule according claim 24, wherein the ProS1 protein comprises (i) the sequence of SEQ ID NO: 6 or (ii) a sequence having sequence identity of at least 85% to the sequence of SEQ ID NO: 6 and comprising a sex hormone-binding globulin (SHBG)-like domain of ProS1 protein, and
wherein the first region does not comprise N-terminal gamma carboxyglutamic acid (GLA) domain and epidermal growth factor (EGF)-like domain.

26. The binding molecule according to claim 24, wherein the ProS1 protein comprises one or more sequences selected from the group consisting of SEQ ID NO: 6, SEQ ID NO: 88, SEQ ID NO: 89, SEQ ID NO: 90, SEQ ID NO: 91, SEQ ID NO: 92, SEQ ID NO: 93, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, SEQ ID NO: 112, and SEQ ID NO: 113.

27. The binding molecule according to claim 24, which is a monomer or multimer.

28. The binding molecule according to claim 24, wherein the target substance is β-Amyloid.

29. The binding molecule according to claim 24, wherein the target substance is soluble amyloid, oligomeric amyloid, aggregated amyloid, or combinations thereof.

30. The binding molecule according to claim 24, wherein the second region that specifically binds to the target substance is selected from the group consisting of an antibody or an antigen-binding fragment thereof, an antibody-like protein, a peptide, an aptamer, and a soluble receptor, which each specifically bind to the target substance.

31. A nucleic acid molecule encoding
(a) the binding molecule according to claim 24, or
(b) a binding molecule comprising a first region capable of binding to a TAM receptor and a second region capable of specifically binding to a target substance, said target substance being a substance of which aberrant accumulation in a living tissue is characteristic of or associated with a disease, wherein the first region and the second region are coupled to each other directly or via a linker, wherein the first region comprises a Gas6 protein comprising (i) the sequence of SEQ ID NO: 5 or (ii) a sequence having sequence identity of at least 85% to the sequence of SEQ ID NO: 5 and comprising a sex hormone-binding globulin (SHBG)-like domain of Gas6 protein, wherein the first region does not comprise N-terminal gamma carboxyglutamic acid (GLA) domain and epidermal growth factor (EGF)-like domain.

32. An expression vector comprising the nucleic acid molecule according to claim 31.

33. A cell expressing the binding molecule according to claim 32.

34. A pharmaceutical composition comprising:
(i) the binding molecule according to according to claim 24, or a binding molecule comprising a first region capable of binding to a TAM receptor and a second region capable of specifically binding to a target substance, said target substance being a substance of which aberrant accumulation in a living tissue is characteristic of or associated with a disease, wherein the first region and the second region are coupled to each other directly or via a linker, wherein the first region comprises a Gas6 protein comprising (i) the sequence of SEQ ID NO: 5 or (ii) a sequence having sequence identity of at least 85% to the sequence of SEQ ID NO: 5 and comprising a sex hormone-binding globulin (SHBG)-like domain of Gas6 protein, wherein the first region does not comprise N-terminal gamma carboxyglutamic acid (GLA) domain and epidermal growth factor (EGF)-like domain,
(ii) a polynucleotide encoding the binding molecule of (i),
(iii) an expression vector carrying the polynucleotide (ii),
(iv) a cell comprising the polynucleotide (ii) or the expression vector (iii), or
(iv) a combination thereof; and
a pharmaceutically acceptable carrier.

35. A method selected from the group consisting of:
(i) reducing or enhancing a reduction of an aberrant deposit of a substance that causes or characterizes a disorder in a subject,
(ii) removing or clearing or enhancing clearance of an aberrant deposit of a substance that causes or characterizes a disorder in a subject,
(iii) suppressing formation of aberrant accumulations of substance in a subject,
(iv) treating or preventing a disease in subject, said disease being associated with or characterized by aberrant accumulation of a substance in a subject,
(v) delaying development of a symptom associated with a disease that is characterized by, associated with, or caused by aberrant accumulation of substance, and
(vi) a combination thereof,
said method comprising administering to the pharmaceutical composition according to claim 34.

36. The method according to claim 35, wherein the substance is soluble amyloid, oligomeric amyloid, aggregated amyloid, or combinations thereof.

37. The method according to claim 35, wherein the aberrant deposit of substance is in a tissue, blood, or brain of the subject.

38. The binding molecule of claim 24, wherein the first region is an anti-Axl antibody or an antigen-binding fragment thereof.


SEQUENCE LISTING (169 sequences)

SEQ ID NO: 1 - protein, 173 aa, synthetic construct
  REGION 1..173: note = TAM(Axl)-binding sequence
GRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQC

SEQ ID NO: 2 - protein, 194 aa, synthetic construct
  REGION 1..194: note = TAM(Axl)-binding sequence
GSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSC

SEQ ID NO: 3 - protein, 177 aa, synthetic construct
  REGION 1..177: note = TAM(Axl)-binding sequence
LLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHC

SEQ ID NO: 4 - protein, 183 aa, synthetic construct
  REGION 1..183: note = TAM(Axl)-binding sequence
YYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSC

SEQ ID NO: 5 - protein, 400 aa, synthetic construct
  REGION 1..400: note = TAM(Axl)-binding sequence
DILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 6 - protein, 393 aa, synthetic construct
  REGION 1..393: note = TAM(Axl)-binding sequence
VVSVCLPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNS

SEQ ID NO: 7 - protein, 678 aa, Homo sapiens
  REGION 1..678: note = GAS6 protein
MAPSLSPGPAALRRAPQLLLLLLAAECALAALLPAREATQFLRPRQRRAFQVFEEAKQGHLERECVEELCSREEAREVFENDPETDYFYPRYLDCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 8 - protein, 173 aa, synthetic construct
  REGION 1..173: note = TAM(Axl)-binding sequence
GRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKANTKMQC

SEQ ID NO: 9 - protein, 173 aa, synthetic construct; identical to SEQ ID NO: 8
  REGION 1..173: note = TAM(Axl)-binding sequence

SEQ ID NO: 10 - protein, 172 aa, synthetic construct
  REGION 1..172: note = TAM(Axl)-binding sequence
GRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKANTRMQ

SEQ ID NO: 11 - protein, 173 aa, synthetic construct
  REGION 1..173: note = TAM(Axl)-binding sequence
GRMFSGIPVIRLRFKRLQPTRLVAEFDFRTFDPEGVLFFAGGHQDSTWIVLALRAGRLELQLRYNGIGRVTSSGPVINHGMWQTISVEELERNLVIKVNKDAVMKIAVAGDLFQLDRGLYHLNLTVGGIPFKEKDLIQPINPRLDGCLRSWNWLNGEDTTIQETVKVNTKMQC

SEQ ID NO: 12 - protein, 173 aa, synthetic construct
  REGION 1..173: note = TAM(Axl)-binding sequence
GRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKANAKMQC

SEQ ID NO: 13 - protein, 173 aa, synthetic construct
  REGION 1..173: note = TAM(Axl)-binding sequence
GRMFSGTPVIRLRFKRLQPTRLVAEFDFRTFDPEGILLFAGGHQDSTWIVLALRAGRLELQLRYSGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIHEAVKVNARMQC

SEQ ID NO: 14 - protein, 171 aa, synthetic construct
  REGION 1..171: note = TAM(Axl)-binding sequence
GRMFSGTPVIRLRFKRLQPTRLVAEFDFRTFDPEGVLFFAGGHQDSAWIVLGLRAGRLELQLRYHGVSRVTSSGPVINHGMWQTISVEELDRNLVVKVNRDAVMKIAVAGDLFQLDRGLYHLNLTVGGIPFKERDLVQPINPRLDGCVRSWNWLNGEDTTIQETVKANPKM

SEQ ID NO: 15 - protein, 173 aa, synthetic construct
  REGION 1..173: note = TAM(Axl)-binding sequence
GRMFSGTPVIRLRFKRLQPTRLVAEFDFRTFDPEGVLFFAGGHQDSTWIVLALRAGRLELQLRYGGVGRVTSSGPVINHGTWQTISVEELERNVVVKVNKDAVMKIAVAGDLFQRDRGLYHLNLTVGGIPFKEKDLVQPINPRLDGCMRSWNWLNGEDSAIQETVRANARMQC

SEQ ID NO: 16 - protein, 173 aa, synthetic construct
  REGION 1..173: note = TAM(Axl)-binding sequence
GRMFSGTPVIRLRFKRLQPTRLLAEFDFRTFDPEGVLFFAGGHQDSAWIVLGLRAGRLELQLRYGGVGRVTSSGPVINHGTWQTISVEELDRNLVIKVNKDAVMKIAVAGDLFQLDRGLYHLNLTVGGIPFKEKDLVQPINPRLDGCMRSWNWLSGEDTTIQETVKANSKMQC

SEQ ID NO: 17 - protein, 173 aa, synthetic construct; identical to SEQ ID NO: 15
  REGION 1..173: note = TAM(Axl)-binding sequence

SEQ ID NO: 18 - protein, 173 aa, synthetic construct
  REGION 1..173: note = TAM(Axl)-binding sequence
GRMFSGTPVIRLRFKRLQPTRLVAEFDFRTFDPEGVLFFAGGHQDSTWIVLGLRAGRLELQLRYQGVGRVTSSGPVINHGMWQTISVEELERNLVIKVNKDAVMKIAVAGDLFQLDRGLYHLNLTVGGIPFKEKDLVQPMNPRLDGCMRSWNWLNGEDTTIQETVKVNVKMQC

SEQ ID NO: 19 - protein, 173 aa, synthetic construct
  REGION 1..173: note = TAM(Axl)-binding sequence
GRMFSGTPVIRLRFKRLQPTRLVAEFDFRTFDPEGVLLFAGGHQDGTWVMLALRAGRLELQLHYNGVGRVTSSGPVINHGAWQTISVEEMARSLVIKVNRDAVMKIAVAGDLFQPERGMFHLNLTVGGIPFHEKDLVQPINPRLDGCIRSWNWMNGEDTTIQETVKVNTKMQC

SEQ ID NO: 20 - protein, 173 aa, synthetic construct; identical to SEQ ID NO: 18
  REGION 1..173: note = TAM(Axl)-binding sequence

SEQ ID NO: 21 - protein, 163 aa, synthetic construct
  REGION 1..163: note = TAM(Axl)-binding sequence
RLRFKRLQPTRLVAEFDFRTFDPEGVLFFAGGHQDSTWIVLGLRAGRLELQLRYQGVGRVTSSGPVINHGMWQTISVEELERNLVIKVNKDAVMKIAVAGDLFQLDRGLYHLNLTVGGIPFKEKDLVQPMNPRLDGCMRSWNWLNGEDTTIQETVKVNVKMQC

SEQ ID NO: 22 - protein, 170 aa, synthetic construct
  REGION 1..170: note = TAM(Axl)-binding sequence
FSGTPVIRLRFKRLQPTRLVAEFDFRTFDPEGVLLFAGGHQDGTWVMLALRAGRLELQLHYNGVGRVTSSGPVINHGAWQTISVEEMARSLVIKVNRDAVMKIAVAGDLFQPERGMFHLNLTVGGIPFHEKDLVQPINPRLDGCIRSWNWMNGEDTTIQETVKVNTKMQC

SEQ ID NO: 23 - protein, 171 aa, synthetic construct
  REGION 1..171: note = TAM(Axl)-binding sequence
MFSGTPVIRLRFKRLQPTRLVAEFDFRTFDPEGVLFFAGGHQDSTWIVLGLRAGRLELQLRYQGVGRVTSSGPVINHGMWQTISVEELERNLVIKVNKDAVMKIAVAGDLFQLDRGLYHLNLTVGGIPFKEKDLVQPMNPRLDGCMRSWNWLNGEDTTIQETVKVNVKMQC

SEQ ID NO: 24 - protein, 194 aa, synthetic construct
  REGION 1..194: note = TAM(Axl)-binding sequence
GSFYPGSGFAFYSLDYMRTPLDIGTESTWEIEVVAHIRPAADTGVLFALWVPDLRAVPLSVALVDYHSTKKLKKQLVVLAVEHVALALMEIKVCDGQEHMVTISLREGEATLEVDGTRGQSEVSAAQLQERLAVLEKHLRSPVLTFAGGLPDVPVTSAPVTAFYRGCMTLEVNRRLLDLDEAAYKHSDITAHSC

SEQ ID NO: 25 - protein, 194 aa, synthetic construct
  REGION 1..194: note = TAM(Axl)-binding sequence
GSFYPGSGFAFYSLDYMRTPLDIGTESTWEIEVVAHIRPAADTGVLFALWVPDLRAVPLSVALVDYHSTKKLKKQLVVLAVEHVALALMEIKVCDGQEHVVTVSLRDGEATLEVDGTRGQSEVSATQLQERLAVLERHLRSPVLTFAGGLPDVPVTSAPVTAFYRGCMTLEVNRRLLDLDEAAYKHSDITAHSC

SEQ ID NO: 26 - protein, 189 aa, synthetic construct
  REGION 1..189: note = TAM(Axl)-binding sequence
GSFYPGSGFAFYSLDYMRTPLDIGTESTWEIEVVAHIRPAADTGVLFALWVPDLRAVPLSVALVDYHSTKKLKKQLVVLAVEHVALALMEIKVCDGQEHVVTVSLRDSEATLEVDGTRGQSEVSATQLQERLAVLERHLRSPVLTFAGGLPDVPVTSAPVTAFYRGCMTLEVNRRLLDLDEAAYKHSDI

SEQ ID NO: 27 - protein, 194 aa, synthetic construct
  REGION 1..194: note = TAM(Axl)-binding sequence
GSFYPGSGFAFYSLDYMRTPLDVGTESAWEIEVVAHIRPAADTGVLFALWVPDLRAVPLSVALVDYHSTKKLKKQLVVLAVEHVALALMEIKVCDGQEHVVTVSLRDGEATLEVDGTRGQSEVSAAQLQERLAVLERHLRSPVLTFAGGLPDVPVTSAPVTAFYRGCMTLEVNRRPLDLDEAAYKHSDITAHSC

SEQ ID NO: 28 - protein, 194 aa, synthetic construct
  REGION 1..194: note = TAM(Axl)-binding sequence
GSFYPGSSFAFYSLDYVRTPLDVGTESTWEIDVVAHIRPAADTGVLFALWVPDLRAVPLSVALVDYHSTKKLKKQLVVLAVEHVALALMEIKVCDGQEHVVTVSLRDGEATLEVDGTRGQSEVSATQLQERLAVLERHLRSPVLTFAGGLPDVPVTSAPVTAFYRGCMTLEVNRRLLDLDEAAYKHGDITAHSC

SEQ ID NO: 29 - protein, 194 aa, synthetic construct
  REGION 1..194: note = TAM(Axl)-binding sequence
GSFYPGSGFAFYSLDYMRTPLDIRTESTWEIEVVAHIRPAADTGVLFALWVPDLRAVPLSVALVDYHSTKKLKKQLVVLAVEHVALALMEIKVCDGQEHMVTISLREGEATLEVDGTRGQSEVSAAQLQERLAVLEKHLQSPVLTFAGGLPDVPVTSAPVTAFYRGCMTLEVNRRLLDLDEAAYKHSDITAHSC

SEQ ID NO: 30 - protein, 190 aa, synthetic construct
  REGION 1..190: note = TAM(Axl)-binding sequence
GSFYPGSGFAFYSLDYMRTPLDIRTESTWEIEVVAHIRPAADTGVLFALWVPDLRAVPLSVALVDYHSTKKLKKQLVVLAVEHVALALMEIKVCDGQEHMVTISLREGEATLEVDGTRGQSEVSAAQLQERLAVLEKHLQSSVLTFAGGLPDVPVTSAPVTAFYRGCMTLEVNRRLLDLDEAAYKHSDIT

SEQ ID NO: 31 - protein, 191 aa, synthetic construct
  REGION 1..191: note = TAM(Axl)-binding sequence
FYPGGFCLPSLDYMRTPLDIGTESTWEIEVVAHIRPAADTGVLFALWVPDLRAVPLSVALVDYHSTKKLKKQLVVLAVEHVALALMEIKVCDGQEHMVTISLREGEATLEVDGTRGQSEVSAAQLQERLAVLEKHLRSPVLTFAGGLPDVPVTSAPVTAFYRGCMTLEVNRRLLDLDEAAYKHSDITAHSC

SEQ ID NO: 32 - protein, 195 aa, synthetic construct
  REGION 1..195: note = TAM(Axl)-binding sequence
GRDRPASHGAQPVSPQAVRTPLDIGTESTWEIEVVAHIRPAADTGVLFALWVPDLRAVPLSVALVDYHSTKKLKKQLVVLAVEHVALALMEIKVCDGQEHMVTVSLREGEATLEVDGTRGQSEVSAAQLQERLAVLEKHLQSPVLTFAGGLPDVPVTSAPVTAFYRGCMTLEVNRRLLDLDEAAYKHSDITAHSC

SEQ ID NO: 33 - protein, 194 aa, synthetic construct
  REGION 1..194: note = TAM(Axl)-binding sequence
GSFYPGNGFAFYSLNYMRTPLDVGTESTWEIEVVAHIRPAADTGVLFALWAADLRAVPLSVALVDYHSTKKLKKQLVVLAVERVALALMEIKVCDGQEHVVTVSLREGEATLAVDGTRGQSEVSAAQLQERLATLHRHLQSPVLTFAGGLPDVPVTSAPVTAFYRGCMTLEVNRRLLDLDEAAYKHGDITSHSC

SEQ ID NO: 34 - protein, 676 aa, Homo sapiens
  REGION 1..676: note = Pro S protein
MRVLGGRCGAPLACLLLVLPVSEANLLSKQQASQVLVRKRRANSLLEETKQGNLERECIEELCNKEEAREVFENDPETDYFYPKYLVCLRSFQTGLFTAARQSTNAYPDLRSCVNAIPDQCSPLPCNEDGYMSCKDGKASFTCTCKPGWQGEKCEFDINECKDPSNINGGCSQICDNTPGSYHCSCKNGFVMLSNKKDCKDVDECSLKPSICGTAVCKNIPGDFECECPEGYRYNLKSKSCEDIDECSENMCAQLCVNYPGGYTCYCDGKKGFKLAQDQKSCEVVSVCLPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNS

SEQ ID NO: 35 - protein, 177 aa, synthetic construct
  REGION 1..177: note = TAM(Axl)-binding sequence
LLYLAEQFAGVVLYLKFRLPEISRFSAEFDFRTYDSEGVILYAESIDHSAWFLIALRGGKIEIQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHC

SEQ ID NO: 36 - protein, 177 aa, synthetic construct
  REGION 1..177: note = TAM(Axl)-binding sequence
LLYLAEQFAGVVLYLKFRLPEISRFSAEFDFRTYDSQGVILYAESIDHSAWLLIALRGGKIEVQLKNEHTSKITTGGDIINNGLWNMVSVEELEHSISIKIAKEAVMDINKPGPLFKPENGLLETKVYFAGFPRKVESELIKPINPRLDGCIRSWNLMKQGASGIKEIIQEKQNKHC

SEQ ID NO: 37 - protein, 177 aa, synthetic construct; identical to SEQ ID NO: 35
  REGION 1..177: note = TAM(Axl)-binding sequence

SEQ ID NO: 38 - protein, 177 aa, synthetic construct; identical to SEQ ID NO: 36
  REGION 1..177: note = TAM(Axl)-binding sequence

SEQ ID NO: 39 - protein, 177 aa, synthetic construct
  REGION 1..177: note = TAM(Axl)-binding sequence
LLYLAEQFAGVVLYLKFRLPEISRFSAEFDFRTYDSQGVILYAESIDHSAWLLIALRGGKIEVQLKNEHTSKITTGGAIINNGLWNMVSVEELEHSISIKIAKEAVMDINKPGPLFKPENGLLETKVYFAGFPRKVESELIKPINPRLDGCIRSWNLMKQGASGIKEIIQEKQNKHC

SEQ ID NO: 40 - protein, 177 aa, synthetic construct
  REGION 1..177: note = TAM(Axl)-binding sequence
LLYLAEQFAGVVLYLKFRLPEISRFTAEFDFRTYDSEGVVLYAESIDHSAWILIAVRDGKFEVQLKNEQTSKITTGGGIINNGVWHTVSVEELEHSVSLKIAKEAVMNINKLGPLFKPEHGFLETKVYFAGFPRKVESQFIKPINPRLDGCIRGWNLMKQGASGVKEIIQEKQNKHC

SEQ ID NO: 41 - protein, 177 aa, synthetic construct
  REGION 1..177: note = TAM(Axl)-binding sequence
LLYLAEQFVGVVLYLKFRLPEISRFSAEFDFRTYDSEGVILYAESLDHSAWFLVALRDGKIEIQFKNEHTTKITTGGKVISNGLWNMVSVEELEHSISVKIAKEAVMNINKPGSLFNPTNGFLETKVYFAGFPRKVENALIKPINPRLDGCIRGWNLMNQGASGVKEIIQEKQNKHC

SEQ ID NO: 42 - protein, 177 aa, synthetic construct; identical to SEQ ID NO: 40
  REGION 1..177: note = TAM(Axl)-binding sequence

SEQ ID NO: 43 - protein, 177 aa, synthetic construct
  REGION 1..177: note = TAM(Axl)-binding sequence
LLYLAEQFAGVVLYLKFRLPEISRFTAEFDFRTYDSEGVVLYAESLDHSAWILIAVRDGKFEVQLQNEQTSRITTGGGVVNNGVWHTVSVEELEHSVSLKIAKEAVMNINKLGPLFKPEHGFLETKVYFAGFPRQVESQFIKPINPRLDGCIRGWNLMKQGASGVKEIIQEKQNKHC

SEQ ID NO: 44 - protein, 182 aa, synthetic construct
  REGION 1..182: note = TAM(Axl)-binding sequence
  VARIANT 42: note = X can be any amino acid
LLYLAEQISGVVLYLKFHLPEISRFSAEFHFWTYDSEGMILXAESVNHSAWLLIALRGGKIEVQLKNEHTSKITTEGDVINNGLWNELSTSQVSVEELEHSISIKIAKEAVMDIDKPGPLFKPENGLLETKVYFAGFPQKVESELIKPINPCLDGCIRGWNLMKQGASGIKEIIQEKQNKHC

SEQ ID NO: 45 - protein, 177 aa, synthetic construct
  REGION 1..177: note = TAM(Axl)-binding sequence
LLYLAEQFVGVVLYLKFRLPEITRFSAEFDFRTYDSEGVILYAESSDHSAWFLIALRDGKIEIQFKNEHTTKITTGGRVINDGLWNMVSVEELEQSISVKIAKEAVMNINKPESLFKPTNGFLETKVYFAGLPRKVENALIKPINPRLDGCIRGWNLMNQGASGVKEIIQEKQNKHC

SEQ ID NO: 46 - protein, 183 aa, synthetic construct
  REGION 1..183: note = TAM(Axl)-binding sequence
YYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAILDKAMKAKVATYLGGLPDVPFSATPVNAFYNGCMEVNINGVQLDLDEAISKHNDIRAHSC

SEQ ID NO: 47 - protein, 183 aa, synthetic construct
  REGION 1..183: note = TAM(Axl)-binding sequence
YYPGSGIAQFHIDYNNVSGAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSC

SEQ ID NO: 48 - protein, 183 aa, synthetic construct
  REGION 1..183: note = TAM(axl)-binding sequence
YYPGSGIAQFHIDYNNVSSAEGWHINVTLNIRPSMGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSC

SEQ ID NO: 49 - protein, 183 aa, synthetic construct
  REGION 1..183: note = TAM(axl)-binding sequence
YYPGSGIAQFHIDYNNVSSAEGWHVNVTLNIRPSTGTGVMLALVSGNNTVPFAVSLVDSTSEKSQDILLSVENTVIYRIQAPSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSC

SEQ ID NO: 50 - protein, 183 aa, synthetic construct
  REGION 1..183: note = TAM(axl)-binding sequence
YYPGSGIAQFHIDYNNVSSAEGWHINVTLNIRPSMGTGVMLALVSGNSTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSC

SEQ ID NO: 51 - protein, 183 aa, synthetic construct
  REGION 1..183: note = TAM(Axl)-binding sequence
YYPGSGIAEFHIDYNNGSNAEGWHINVTLNIRPSMGTGVMLALVSSNNTVPFAVSLVDSTSEKSQDIVLSVENTVIYRIQALSLCSNQRSHLEFRVNRNNLELLTPLKIETISQEELQTQLAILDKAMKGKVATYLGGLPDVPFSATPVNAFYNGCMEVNVNGVELDLDEAISKHNDIRAHSC

SEQ ID NO: 52 - protein, 183 aa, synthetic construct
  REGION 1..183: note = TAM(Axl)-binding sequence
  VARIANT 80: note = X can be any amino acid
YYPGFGIAQFHVDYNNVSSAEGWHINVTLNIHPSMGTGVMLALVSGNNTVPFAVSLVDSTSEKSQDILLSVENTVIYRIXALSLCSDQQSHLEFRVNRNNLELLIPLKIETISHEDLQRQLAILDKAMKAKVATYLGGLPDVPFSATPVNAFYNGCMEVNINGVQLDLDEAISKHNDIRAHSC

SEQ ID NO: 53 - protein, 183 aa, synthetic construct
  REGION 1..183: note = TAM)Ax)-binding sequence
YYPGSGIAQFHIDYNNGSNAEGWHINVTLNIRPSMGTGVMLALVSSNNTVPFAVSLVDSTSEKSQDIVLSVENTVIYRIQALSLCSNQRSHLEFRVNRNNLELLTPLKIETISHEELQRQLAILDKAMTGKVATYLGGLPDVPFSATPVNAFYNGCMEVNINGVQLDLDEAISKHNDIRAHSC

SEQ ID NO: 54 - protein, 183 aa, synthetic construct
  REGION 1..183: note = TAM(Axl)-binding sequence
YYPGSGIAQFHIDYNNGSNAEGWHINVTLNIRPSMGTGVMLALVSGNNTVPFAVSLVDSTSEKSQDIVLSVENTVIYRIQALSLCSNQRSHLEFRVNRNNLELLTPLKIETISDEELRRQLAILDKAMTGKVATYLGGLPDVPFSATPVNAFYNGCMEVNINDVQLDLDEAISKHNDIRAHSC

SEQ ID NO: 55 - protein, 183 aa, synthetic construct
  REGION 1..183: note = TAM(Axl)-binding sequence
YYPGSGIAEFHIDYNNGSNAEGWHINVTLNIRPSMGTGVMLALVSSNNTVPFAVSLVDSTSEKSQDIVLSVENTVIYRIQALSLCSNQRSHLEFRANRNNLELLTPLKIETISQEELQTQLAILDKAMKGKVATYLGGLPDVPFSATPVNAFYNGCMEVNINGVELDLDEAISKHNDIRAHSC

SEQ ID NO: 56 - protein, 183 aa, synthetic construct
  REGION 1..183: note = TAM(Axl)-binding sequence
  VARIANT 80: note = X can be any amino acid
YYPSFGIAQFRIDYNNVSSVEGWHINVTLNIHPSMGTGVMLALVSGNNTVPFAVSLVDSTSEKSQDILLSVENTVIYLIXALSLCSDQQSHLEFIVNRNNLELLTPLKIETISHEDLQRQLAILDKAMKAKVATYLGGLPDVPFSATPVNALYKGCMEVNINGVQLDLDEAISKHNDIIAHSC

SEQ ID NO: 57 - protein, 183 aa, synthetic construct
  REGION 1..183: note = TAM(Axl)-binding sequence
YYPGSGIAKFIVDYNNVSSAEGWYVNVSLNIRPSKGTGVMLALVSHNNTVPFAVSLVDSTSEKLQDILLSVEKTVIYRIQALSLCSDQQFHLEFKVNRHNLEVSTPLKMETISHEDLQKQLAILDKAMQGEVVTYLGGLPDVPFSATPVNAFYNGCMEVNINGVLLDLDEAISKHNDIRAHSC

SEQ ID NO: 58 - protein, 183 aa, synthetic construct
  REGION 1..183: note = TAM(Axl)-binding sequence
YYPGSGIAKFIIDYNNASNAEGWYVNVSLNIRPSTGTGVMLALVSRNNTVPFAVSLVDSTSEKLQDILLSVEKTVVCRIQALSLCSDQQSHLEFKVNRHNLEVLTPLKMETISHEDLQKQLAILDKAMQGDVVTYLGGLPDVPFSATPVNAFYNGCMEVNINGVLLDLDEAISKHNDIRAHSC

SEQ ID NO: 59 - protein, 183 aa, synthetic construct
  REGION 1..183: note = TAM(Axl)-binding sequence
YYPGSGIAKFIIDYNNVSSAEGWHVNVSLNIRPSMGTGVMLALVSRNNTVPFAVSLVDSTSEKLQDILLSVEKTVIYRIEALSLCSDQQSHLELKVNRHSLEVSTPLKMETVSHEDIQKELAILDKAMQGEVVTYLGGLPDVSFSATPANAFYNGCMEVNMNGVLLDLDEAISKHNDIRAHSC

SEQ ID NO: 60 - protein, 176 aa, synthetic construct
  REGION 1..176: note = TAM(Axl)-binding sequence
SGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRA

SEQ ID NO: 61 - protein, 173 aa, synthetic construct
  REGION 1..173: note = TAM(Axl)-binding sequence
HIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSC

SEQ ID NO: 62 - protein, 173 aa, synthetic construct
  REGION 1..173: note = TAM(Axl)-binding sequence
YYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAIS

SEQ ID NO: 63 - protein, 395 aa, synthetic construct
  REGION 1..395: note = TAM(Axl)-binding sequence
VPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 64 - protein, 390 aa, synthetic construct
  REGION 1..390: note = TAM(Axl)-binding sequence
DILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAH

SEQ ID NO: 65 - protein, 400 aa, synthetic construct
  REGION 1..400: note = TAM(Axl)-binding sequence
DILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNKDAVMKIAVAGDLFQPERGLYHLNLTVGGIPFHEKDLVQPINPRLDGCMRSWNWLNGEDTTIQETVKANTRMQCFSVTERGSFYPGSGFAFYSLDYMRTPLDIGTESTWEIEVVAHIRPAADTGVLFALWVPDLRAVPLSVALVDYHSTKKLKKQLVVLAVEHVALALMEIKVCDGQEHVVTVSLRDGEATLEVDGTRGQSEVSATQLQERLAVLERHLRSPVLTFAGGLPDVPVTSAPVTAFYRGCMTLEVNRRLLDLDEAAYKHSDITAHSCPPVEPAAA

SEQ ID NO: 66 - protein, 400 aa, synthetic construct
  REGION 1..400: note = TAM(Axl)-binding sequence
DILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMQTPLDIGTESTWEIEVVAHIRPAADTGVLFALWVPDLRAVPLSVALVDYHSTKKLKKQLVVLAVEHVALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPTTA

SEQ ID NO: 67 - protein, 380 aa, synthetic construct
  REGION 1..380: note = TAM(Axl)-binding sequence
RMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 68 - protein, 400 aa, synthetic construct
  REGION 1..400: note = TAM(Axl)-binding sequence
DILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESAWEIEVVAHIRPAADTGVLFALWVPDLRAVPLSVALVDYHSTKKLKKQLVVLAVEHVALALMEIKVCDGQEHVVTVSLRDGEATLEVDGTRGQSEVSAAQLQERLAVLERHLRSPVLTFAGGLPDVPVTSAPVTAFYRGCMTLEVNRRPLDLDEAAYKHSDITAHSCPPVEPAAA

SEQ ID NO: 69 - protein, 400 aa, synthetic construct
  REGION 1..400: note = TAM(Axl)-binding sequence
DILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKANTRMQCFSVTERGSFYPGSGFAFYSLDYMRTPLDIGTESTWEIEVVAHIRPAADTGVLFALWVPDLRAVPLSVALVDYHSTKKLKKQLVVLAVEHVALALMEIKVCDGQEHVVTVSLRDSEATLEVDGTRGQSEVSATQLQERLAVLERHLRSPVLTFAGGLPDVPVTSAPVTAFYRGCMTLEVNRRLLDLDEAAYKHSDITAHSCPPVEPAAA

SEQ ID NO: 70 - protein, 400 aa, synthetic construct; identical to SEQ ID NO: 66
  REGION 1..400: note = TAM(Axl)-binding sequence

SEQ ID NO: 71 - protein, 395 aa, synthetic construct
  REGION 1..395: note = TAM(Axl)-binding sequence
LPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEP

SEQ ID NO: 72 - protein, 400 aa, synthetic construct
  REGION 1..400: note = TAM(Axl)-binding sequence
DILPCVPFSMAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKANTKMQCFSVTERGSFYPGSGFAFYSLDYMRTPLDIGTESTWEIEVVAHIRPAADTGVLFALWVPDLRAVPLSVALVDYHSTKKLKKQLVVLAVEHVALALMEIKVCDGQEHMVTISLREGEATLEVDGTRGQSEVSAAQLQERLAVLEKHLRSPVLTFAGGLPDVPVTSAPVTAFYRGCMTLEVNRRLLDLDEAAYKHSDITAHSCPPVEPAAA

SEQ ID NO: 73 - protein, 400 aa, synthetic construct
  REGION 1..400: note = TAM(Axl)-binding sequence
DILPCVPFSMAKSVKSLYLGRMFSGTPVIRLRFKRLQPTRLVAEFDFRTFDPEGILLFAGGHQDSSWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKANTKMQCFSVTERGSFYPGSGFAFYSLDYMRTPLDIGTESTWEIEVVAHIRPAADTGVLFALWVPDLRAVPLSVALVDYHSTKKLKKQLVVLAVEHVALALMEIKVCDGQEHMVTISLREGEATLEVDGTRGQSEVSAAQLQERLAVLEKHLRSPVLTFAGGLPDVPVTSAPVTAFYRGCMTLEVNRRLLDLDEAAYKHSDITAHSCPPVEPAAA

SEQ ID NO: 74 - protein, 400 aa, synthetic construct
  REGION 1..400: note = TAM(Axl)-binding sequence
DILPCVPFSMAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKANPKMQCFSVTERGSFYPGSGFAFYSLDYMRTPLDIGTESTWEIEVVAHIRPAADTGVLFALWVPDLRAVPLSVALVDYHSTKKLKKQLVVLAVEHVALALMEIKVCDGQEHMVTISLREGEATLEVDGTRGQSEVSATQLQERLAVLEKHLRSPVLTFAGGLPDVPLTSAPVTAFYRGCMTLEVNRRLLDLDEAAYKHSDITAHSCPPVEPTAA

SEQ ID NO: 75 - protein, 340 aa, synthetic construct
  REGION 1..340: note = TAM(Axl)-binding sequence
DILPCVPFSLAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDQETVKANAKMQCFSVTERGSFYPGSGFAFYSLDYMRTPLDIRTESTWEIEVVAHIRPAADTGVLFALWVPDLRAVPLSVALVDYHSTKKLKKQLVVLAVEHVALALMEIKVCDGQEHMVTISLREGEATLEVDGTRGQSEVSAAQLQERLAVLEKHLQSPVLTFAGGLPDVSVTSAPVTAFYRGCMTLEVNRRLLDLDEAAYKHSDITAHSCPPVEPATA

SEQ ID NO: 76 - protein, 400 aa, synthetic construct
  REGION 1..400: note = TAM(Axl)-binding sequence
  VARIANT 100: note = X can be A or T
DILPCVPFSVAKSVKSLYLGRMFSGTPVIRLRFKRLQPTRLVAEFDFRTFDPEGVLLFAGGHQDGTWVMLALRAGRLELQLHYNGVGRVTSSGPVINHGXWQTISVEEMARSLVIKVNRDAVMKIAVAGDLFQPERGMFHLNLTVGGIPFHEKDLVQPINPRLDGCIRSWNWMNGEDTTIQETVKVNTKMQCFSVTERGSFYPGNGFAFYSLNYMRTPLDVGTESTWEIEVVAHIRPAADTGVLFALWAADLRAVPLSVALVDYHSTKKLKKQLVVLAVERVALALMEIKVCDGQEHVVTVSLREGEATLAVDGTRGQSEVSAAQLQERLATLERHLQSPVLTFAGGLPDVPVTSAPVTAFYRGCMTLEVNRRLLDLDEAAYKHGDITSHSCPTVEPAAA

SEQ ID NO: 77 - protein, 400 aa, synthetic construct
  REGION 1..400: note = TAM(Axl)-binding sequence
DILPCVPFSMAKSVKSLYLGRMFSGTPVIRLRYRRLQPTRLVAEFDFRTFDPEGVLLFAGGHQDGTWIMLALRAGRLELQLRYNGVGRITSSGPVINHGTWQTISVEELARSLVIKVNRDAVMKIAVAGDLFQPERGMFHLNLTVGGIPFHEEDLVQPINPRLDGCIRSWNWMNGEDTTIQETVKVNSKMQCFSVTERGSFYPGNGFAFYSLNYMRTPLDVGTESTWEIEVVAHIRPAADTGVLFALWAADLRAVPLSVALVDYHSTKKLKKQLVVLAVERVALALMEIKVCDGQEHVVTVSLREGEATLAVDGTRGQSEVSAAQLQERLATLERHLQSPVLTFAGGLPDVPVTSAPVTAFYRGCMTLEVNRRLLDLDEAAYKHGDITSHSCPTVEPAAA

SEQ ID NO: 78 - protein, 399 aa, synthetic construct
  REGION 1..399: note = TAM(Axl)-binding sequence
DILPCVPFSVAKSLKSLYLGRMFSGTPVIRLRFKRLQPTRLVAEFDFRTFDPEGILFFAGGHRDSTWIVLALRAGRLELQLLFNGVGRVTSSGPVINHGMWQTVSVEELERNLVVKVNKDAVMKIAVPGDLFQLDRGLYHLNLTVGGIPFKEKDLVQPINPRLDGCMRSWNWLNGEDTTIQETVKANAKMQCFSLTEKGSFFPGSGFAFYSLGYVRTSLDVGTETTWEIEVEARIRPAADTGVLLALWAPDHRAVPLSVALVDYHSTKKLKKQLVVLAVESVALALMEIKVCDGQEHVVSVSVREDEATLEVDGTKGQSEVSAAQLQERLAALGRHLRDPVLTFIGGLPEVPVTSAPVTAFYRGCMTLEVNRRPLDLDEASYKHSDITAHSCPPVEPAA

SEQ ID NO: 79 - protein, 399 aa, synthetic construct
  REGION 1..399: note = TAM(Axl)-binding sequence
DILPCVPFGVAKSVKSLYLGRMFSGTPVIRLRFKRLQPTRLVAEFDFRTFDPEGVLFFAGGRQDSTWIVLALRAGRLELQLRYNGVGRVTSSGPVINHGTWQTISVEELERNLVVKVNKDAVMKIAVAGDLFQRDRGLYHLNLTVGGIPFKEKELVQPINPRLDGCMRSWNWLNSEDTIIQETVKVNTKMQCFSVTEKGSFYPGTGFAFYSLNYMRTSLDTGTETTWEIKVMARIRPATDTGVLLALCAPDHRTVPLSVALVDYHSTKKLKKQLVVLAVESVVLALVEIKACDGQEHEVSVSLKEGEATLEVDGTRGWSEASATQLQERLDTLRRHLHDPVLTFTGGLPDVPVTAAPVTAFYRGCMTLEVNRRLLDLDEAAYKHSDITSHSCPPVEPTA

SEQ ID NO: 80 - protein, 399 aa, synthetic construct
  REGION 1..399: note = TAM(Axl)-binding sequence
DILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAA

SEQ ID NO: 81 - protein, 398 aa, synthetic construct
  REGION 1..398: note = TAM(Axl)-binding sequence
DILPCVPFSVAKSMKSLYLGRMFSGTPVIRLRFKRLQPTRLVAEFDFRTFDPEGVLFFAGGHQDSTWIVLALRAGRLELQLHYNGVGRVTSSGPVINHGMWQTISVEELERNVVIKVNKDAVMKIAVAGDLFQLDRGLYHLNLTVGGIPFKEKHLVQPINPRLDGCMRSWSWLNGEDTTIQETVKVNTRMQCFSVTERGSFYPGNGLAIYSLNYSRTPPDVGTETAWEVEVVARIRPATDTGVLLALVGDGHTVPLSVALVDYHSTKKLKKQLVVLAVEGMALALMEIKVCDGQEHVVAVSLKAGEASLEVDGTKGQSEVSAAQLQERLAVLGRHLQGPLLTFIGGLPDVPVTSAPVTAFYRGCMTLEVNQKRLDLDEATYKHSDITSHSCPPVEHAA

SEQ ID NO: 82 - protein, 397 aa, synthetic construct
  REGION 1..397: note = TAM(Axl)-binding sequence
DILPCVPFTMAKSVKSLYLGRMFSGTPVIRLRFKRLQPTRLLAEFDFRTFDPEGVLFFAGGRSDSTWIVLGLRAGRLELQLRYNGVGRITSSGPVINHGMWQTISVEELERNLVIKVNKDAVMKIAVAGELFQLERGLYHLNLTVGGIPFKEQDLVQPINPRLDGCMRSWNWLNGEDSAIQETVKANTKMQCFSVTERGSFFPGGAFAFYSLNYTRTSLDVGTETTWEVEIVAWIRPATDTGVLLALVGDNHVVPLSVALVDYHSTKKLKKQLVVLAVEDVALALMEIKVCDSQEHVVTVSLRDGEATLEVDGTKGQSEVSAAQLQERLDTLGEHLQGTVLTFVGGLPEVPVTSAPVTAFYRGCMTLEVNQKTLDLDEASYKHSDITSHSCPPVEHA

SEQ ID NO: 83 - protein, 398 aa, synthetic construct
  REGION 1..398: note = TAM(Axl)-binding sequence
DILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPA

SEQ ID NO: 84 - protein, 398 aa, synthetic construct
  REGION 1..398: note = TAM(Axl)-binding sequence
DILPCVPFNVAKSVKSLYLGRMFSGTPVIRLRFKRLQPTRLLAEFDFRTFDPEGVLFFAGGRQDSTWIVLGLRAGRLELQLRYHGVGRVTSSGPVINHGMWQTISVEELDRNLVIKVNKDAVMKIAVAGDLFQLDRGLYHLNLTVGGIPFKEKDLVQPINPRLDGCMRSWNWLSGEDTTIQETVKANSKMQCFSVTERGSFYPGTGFAFYSLDYTRTSTAVGTEATWEIEVVAQIRPATDTGVLLALVAGDHVVALSVALVDYHSTKKLKKQLVVLAVEGVTLALMEIKVCDGQEHRVAVSVRKGEATLEVDGTRGQSEVTAAGLQESLAVLGRHLQASVLTFVGGLPDVPVTSAPVTAFYRGCMTLEVNQQALDLDEAAYKHSDITSHSCPPVEQAG

SEQ ID NO: 85 - protein, 395 aa, synthetic construct
  REGION 1..395: note = TAM(Axl)-binding sequence
DILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPV

SEQ ID NO: 86 - protein, 394 aa, synthetic construct
  REGION 1..394: note = TAM(Axl)-binding sequence
DILPCVPFNVAKSVKSLYLGRMFSGTPVIRLRFKRLQPTRLVAEFDFRTFDPEGVLFFAGGHQDSTWIVLALRAGRLELQLHYNGIGRVTSSGSVINHGMWQTISVEELERNLVIKINKDAVMKIAVAGDLFQLDRGLYHLNLTVGGIPFKDKDLVQPINPRLDGCMRSWNWLNGEDSTIQDTVKVNTKMQCFSVTEKGSFYPGNGFAFFSLNYVRTSLDVGTEMTWEIEVIARIRPATDTGVLLALVAKNHTIPLSVALVDYHSTKKLKKQLVILAIENVALSLMEIKVCDGQEHVVTVSVKEGEATLVVDGTKGQSEVSPTQLQERLAVLKRHLQDSVHTFVGGVPDVPVTSAPVTAFYHGCMTLEVNKKALDLDEAVYKHSDITSHSCPPI

SEQ ID NO: 87 - protein, 398 aa, synthetic construct
  REGION 1..398: note = TAM(Axl)-binding sequence
DILPCVPFNVAKSVKSLYLGRMFSGTPVIRLRFKRLQPTRLLAEFDFRTFDPEGVLFFAGGHQDSAWIVLGLRAGRLELQLRYGGVGRVTSSGPVINHGTWQTISVEELDRNLVIKVNKDAVMKIAVAGDLFQLDRGLYHLNLTVGGIPFKEKDLVQPINPRLDGCMRSWNWLSGEDTTIQETVKANSKMQCFSVTERGSFYPGTGFAFYSLDYTRTSAAVGTEAAWEIEVVAWIRPATDTGVLLALVGGDRVVALSVALVDYHSTKKLKKQLVVLAVEGVTLALMEIKVCDGQEHRVAVSVQKGEVTLEVDGTRGQSEVSAAGLQESLAVLERHLQGSVLTFVGGLPDVPVTSAPVTAFYRGCMTLEVNQQTLDLDEAAYKHSDITSHSCPPVEQAS

SEQ ID NO: 88 - protein, 392 aa, synthetic construct
  REGION 1..392: note = TAM(Axl)-binding sequence
VSVCLPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNS

SEQ ID NO: 89 - protein, 388 aa, synthetic construct
  REGION 1..388: note = TAM(Axl)-binding sequence
LPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNS

SEQ ID NO: 90 - protein, 392 aa, synthetic construct
  REGION 1..392: note = TAM(Axl)-binding sequence
VVSVCLPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKN

SEQ ID NO: 91 - protein, 390 aa, synthetic construct
  REGION 1..390: note = TAM(Axl)-binding sequence
VVSVCLPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKT

SEQ ID NO: 92 - protein, 393 aa, synthetic construct
  REGION 1..393: note = TAM(Axl)-binding sequence
VVSVCLPLNLDTKYELLYLAEQFAGVVLYLKFRLPEISRFSAEFDFRTYDSEGVILYAESIDHSAWFLIALRGGKIEIQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLITVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAILDKAMKAKVATYLGGLPDVPFSATPVNAFYNGCMEVNINGVQLDLDEAISKHNDIRAHSCPSVWKKTKNS

SEQ ID NO: 93 - protein, 393 aa, synthetic construct
  REGION 1..393: note = TAM(Axl)-binding sequence
VVSVCLPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHINVTLNIRPSMGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNS

SEQ ID NO: 94 - protein, 393 aa, synthetic construct; identical to SEQ ID NO: 92
  REGION 1..393: note = TAM(Axl)-binding sequence

SEQ ID NO: 95 - protein, 393 aa, synthetic construct
  REGION 1..393: note = TAM(Axl)-binding sequence
VVSVCLPLNLDTKYELLYLAEQFAGVVLYLKFRLPEISRFSAEFDFRTYDSEGVILYAESIYHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNS

SEQ ID NO: 96 - protein, 393 aa, synthetic construct
  REGION 1..393: note = TAM(Axl)-binding sequence
VVSVCLPLNLDTKYELLYLAEQFAGVVLYLKFRLPEISRFSAEFDFRTYDSEGVILYAESIDHSAWFLIALRGGKIEIQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLITVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETTSHEDLQRQLAILDKAMKAKVATYLGGLPDVPFSATPVNAFYNGCMEVNINGVQLDLDEAISKHNDIRAHSCPSVWKKTKNS

SEQ ID NO: 97 - protein, 393 aa, synthetic construct; identical to SEQ ID NO: 92
  REGION 1..393: note = TAM(Axl)-binding sequence

SEQ ID NO: 98 - protein, 393 aa, synthetic construct
  REGION 1..393: note = TAM(Axl)-binding sequence
VVSVCLPLNLDTKYELLYLAEQFAGVVLYLKFRLPEISRFSAEFDFRTYDSEGVILYAESVDHSAWLLIALRGGKIEVQLKNEHTSKITTGGDVINNGLWNMVSVEELEHSISIKIAKEAVMDINKPGPLFKPENGLLETKVYFAGFPQKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQAPSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNS

SEQ ID NO: 99 - protein, 393 aa, synthetic construct
  REGION 1..393: note = TAM(Axl)-binding sequence
VVSVCLPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHINVTLNIRPSMGTGVMLALVSGNSTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNS

SEQ ID NO: 100 - protein, 393 aa, synthetic construct
  REGION 1..393: note = TAM(Axl)-binding sequence
VVSVCLPLNLNTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSGAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNS

SEQ ID NO: 101 - protein, 393 aa, synthetic construct
  REGION 1..393: note = TAM(Axl)-binding sequences
AVSVCLPLNLDTKYELLYLAEQFAGVVLYLKFRLPEISRFSAEFDFRTYDSQGVILYAESIDHSAWLLIALRGGKIEVQLKNEHTSKITTGGDIINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNGSNAEGWHINVTLNIRPSMGTGVMLALVSSNNTVPFAVSLVDSTSEKSQDIVLSVENTVIYRIQALSLCSNQRSHLEFRVNRNNLELLTPLKIETISHEELQRQLAILDKAMTGKVATYLGGLPDVPFSATPVNAFYNGCMEVNINGVQLDLDEAISKHNDIRAHSCPSVWKKTKNS

SEQ ID NO: 102 - protein, 393 aa, synthetic construct
  REGION 1..393: note = TAM(Axl)-binding sequence
AVSVCLPLNLDTKYELLYLAEQFAGVVLYLKFRLPEISRFSAEFDFRTYDSQGVILYAESIDHSAWLLIALRGGKIEVQLKNEHTSKITTGGDIINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYDNGSSAEGWHINVTLNIRPSMGTGVMLALVSSNNTVPFAVSLVDSTSEKSQDIVLSVENTVIYRIQALSLCSNQRSHLEFRVNRNNLELLTPLKIETISHEELQRQLAILDKAMTGKVATYLGGLPDVPFSATPVNAFYNGCMEVNINGVQLDLDEAISKHNDIRAHSCPSVWKKTKNS

SEQ ID NO: 103 - protein, 393 aa, synthetic construct
  REGION 1..393: note = tAM(Axl)-binding sequence
AVSVCLPLNLDTKYELLYLAEQFAGVVLYLKFRLPEISRFSAEFDFRTYDSQGVILYAESIDHSAWLLIALRGGKIEVQLKNEHTSKITTGGDIINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAEFHIDYNNGSNAEGWHINVTLNIRPSMGTGVMLALVSSNNTVPFAVSLVDSTSEKSQDIVLSVENTVIYRIQALSLCSNQRSHLEFRVNRNNLELLTPLKIETISQEELQTQLAILDKAMKGKVATYLGGLPDVPFSATPVNAFYNGCMEVNINGVELDLDEAISKHNDIRAHSCPSVWKKTKNS

SEQ ID NO: 104 - protein, 393 aa, synthetic construct
  REGION 1..393: note = TAM(Axl)-binding sequence
AVSVCLPLNLDTKYELLYLAEQFAGVVLYLKFRLPEISRFSAEFDFRTYDSQGVILYAESIDHSAWLLIALRGGKIEVQLKNEHTSKITTGGDIINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAEFHIDYNNGSNAEGWHINVTLNIRPSMGTGVMLALVSSNNTVPFAVSLVDSTSEKSQDIVLSVENTVIYRIQALSLCSNQRSHLEFRVNRNNLELLTPLKIETISQEELQTQLAILDKAMKGKVATYLGGLPDVPFSATPVNAFYNGCMEVNINGVELDLDEAISKHNDIRAHSCPSIWKKTKNS

SEQ ID NO: 105 - protein, 393 aa, synthetic construct
  REGION 1..393: note = TAM(Axl)-binding sequence
AVSVCLPLNLDTKYELLYLAEQFAGVVLYLKFRLPEISRFSAEFDFRTYDSQGVILYAESIDHSAWLLIALRGGKIEVQLKNEHTSKITTGGDIINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAEFHIDYNNGSNAEGWHINVTLNIRPSMGTGVMLALVSSNNTVPFAVSLVDSTSEKSQDIVLSVENTVIYRIQALSLCSNQRSHLEFRANRNNLELLTPLKIETISQEELQTQLAILDKAMKGKVATYLGGLPDVPFSATPVNAFYNGCMEVNINGVELDLDEAISKHNDIRAHSCPSIWKKTKNS

SEQ ID NO: 106 - protein, 393 aa, synthetic construct
  REGION 1..393: note = TAM(Axl)-binding sequence
AVSVCLPLNLDTKYELLYLAEQFAGVVLYLKFRLPEISRFSAEFDFRTYDSQGVILYAESIDHSAWLLIALRGGKIEVQLKNEHTSKITTGGDIINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAEFHIDYNNGSNAEGWHINVTLNIRPSMDTGVMLALVSSNSTVPFAVSLVDSTSEKSQDIVLSVENTVIYRIQALSLCSSQRSHLEFRVNRNNLELLTPLKIETISHEELQTQLAILDKAMKGKVATYLGGLPDVPFSATPVNAFYNGCMEVNINGVELDLDEAISKHNDIRAHSCPSVWEKTKNS

SEQ ID NO: 107 - protein, 393 aa, synthetic construct
  REGION 1..393: note = TAM(Axl)-binding sequence
AVSVCLPLDLDTKYELLYLAEQFAGVVLYLKFRLPEISRFSAEFDFRTYDSQGVILYAESIDHSAWLLIALRGGKIEVQLKNEHTSKITTGGAIINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAEFHIDYNNGSNAEGWHINVTLNIRPSMGTGVMLALVSSNNTVPFAVSLVDSTSEKSQDIVLSVENTVIYRIQALSLCSNQRSHLEFRVNRNNLELLTPLKIETISQEELQTQLAILDKAMKGKVATYLGGLPDVPFSATPVNAFYNGCMEVNINGVELDLDEAISKHNDIRAHSCPSVWKKTKNS

SEQ ID NO: 108 - protein, 398 aa, synthetic construct
  REGION 1..398: note = TAM(Axl)-binding sequence
  VARIANT 57: note = X can be any amino acid
  VARIANT 285: note = X can be any amino acid
VVSVCLPLNLDTQYELLYLAEQISGVVLYLKFHLPEISRFSAEFHFWTYDSEGMILXAESVNHSAWLLIALRGGKIEVQLKNEHTSKTTTEGDVINNGLWNELSTSQVSVEELEHSISIKIAKEAVMDIDKPGPLFKPENGLLETKVYFAGYPQKVESELIKPINPCLDGCIRGWNLMKQGASGIKEIIQEKQNKHCLVTVEKGSYYPGFGIAQFHVDYNNVSSAEGWHINVTLNIHPSMGTGVMLALVSGNNTVPFAVSLVDSTSEKSQDILLSVENTVIYRIXALSLCSDQQSHLEFRVNRNNLELLIPLKIETISHEDLQRQLAILDKAMKAKVATYLGGLPDVPFSATPVNAFYNGCMEVNINGVQLDLDEAISKHNDIRAHSCPSFWKKTKNS

SEQ ID NO: 109 - protein, 393 aa, synthetic construct
  REGION 1..393: note = TAM(Axl)-binding sequence
AVPVCLPLNLDTKSELLYLAEQFAGVVLYLKFRLPEISRFTAEFDFRTYDSEGVVLYAESIDHSAWILIAVRDGKFEVQLKNEQTSKITTGGGIINNGVWHTVSVEELEHSVSLKIAKEAVMNINKLGPLFKPEHGFLETKVYFAGFPRKVESQFIKPINPRLDGCIRGWNLMKQGASGVKEIIQEKQNKHCLVTVEKGSYYPGSGIAKFIIDYNNVSSAEGWYVNVSLNIRPSMGTGVMLALVSHNNTVPFAVSLVDSTSEKSQDILLSVEKTVVYRIQALSLCSDQQSHLEFRVNRRNLEVSTPLKMETISHEDLQKQLAILDKAMQGEVVTYLGGLPDVPFSAAPANAFYNGCMEVNINGVLLDLDEAISKHNDIRAHSCPSVWKKTKSS

SEQ ID NO: 110 - protein, 416 aa, synthetic construct
  REGION 1..416: note = TAM(Axl)-binding sequence
  VARIANT 57: note = X can be any amino acid
  VARIANT 303: note = X can be any amino acid
VVSVCLPLNLDTQYELFYLAEQFAGVVLYLKFHLPEISRFSAEFHFWTYDSEGMILXAESVNHSAWLLIALRGGKIEVQLENEHTSKITTGGDVINNGLWNVFKIITFLKNVMNAKIVQQIFCVYVSVEELEHSISIKIAKEAVMDINKPGPLFKPENGLLETKVYFAGFPQKAEGELIKPINPCLDGCIRGWNLMKQGASGIKEIIQEKQNKHCLVTVEKGSYYPSFGIAQFRIDYNNVSSVEGWHINVTLNIHPSMGTGVMLALVSGNNTVPFAVSLVDSTSEKSQDILLSVENTVIYLIXALSLCSDQQSHLEFIVNRNNLELLTPLKIETISHEDLQRQLAILDKAMKAKVATYLGGLPDVPFSATPVNALYKGCMEVNINGVQLDLDEAISKHNDIIAHSCPSFWKKTKNS

SEQ ID NO: 111 - protein, 393 aa, synthetic construct
  REGION 1..393: note = TAM(Axl)-binding sequence
AVPVCLLLNLDTKSELLYLAEQFAGVVLYLKFRLPEISRFTAEFDFRTYDSEGVVLYAESIDHSAWILIAVRDGKFEVQLKNEQTSKITTGGGIINNGVWHTVSVEELEHSVSLKIAKEAVMNINKLGPLFKPEHGFLETKVYFAGFPRKVESQFIKPINPRLDGCIRGWNLMKQGASGVKEIIQEKQNKHCLVTVEKGSYYPGSGIAKFIIDYNNASNAEGWYVNVSLNIRPSTGTGVMLALVSRNNTVPFAVSLVDSTSEKLQDILLSVEKTVVYRIQALSLCSDQQSHLEFKVNRHNLEVSTPLKMETISHEDLQKQLAILDKAMQGDVVTYLGGLPDVPFSATPVNAFYNGCMEVNINGVLLDLDEAISKHNDIRAHSCPSVWKKTKSS

SEQ ID NO: 112 - protein, 393 aa, synthetic construct
  REGION 1..393: note = TAM(Axl)-binding sequence
AIPVCLPLNLDTKSELLYLAEQFAGVVLYLKFRLPEISRFTAEFDFRTYDSEGVVLYAESLDHSAWILIAVRDGKFEVQLQNEQTSRITTGGGVVNNGVWHTVSVEELEHSVSLKIAKEAVMNINKLGPLFKPEHGFLETKVYFAGFPRQVESQFIKPINPRLDGCIRGWNLMKQGASGVKEIIQEKQNKHCLVTVEKGSYYPGSGIAKFIIDYNNVSSAEGWHVNVSLNIRPSMGTGVMLALVSRNNTVPFAVSLVDSTSEKLQDILLSVEKTVIYRIEALSLCSDQQSHLELKVNRHSLEVSTPLKMETVSHEDIQKELAILDKAMQGEVVTYLGGLPDVSFSATPANAFYNGCMEVNMNGVLLDLDEAISKHNDIRAHSCPSVWRKTKSS

SEQ ID NO: 113 - protein, 388 aa, synthetic construct
  REGION 1..388: note = TAM(Axl)-binding sequence
VVSVCLPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWK

SEQ ID NO: 114 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 115 - protein, 888 aa, Mus musculus
MGRVPLAWWLALCCWGCAAHKDTQTEAGSPFVGNPGNITGARGLTGTLRCELQVQGEPPEVVWLRDGQILELADNTQTQVPLGEDWQDEWKVVSQLRISALQLSDAGEYQCMVHLEGRTFVSQPGFVGLEGLPYFLEEPEDKAVPANTPFNLSCQAQGPPEPVTLLWLQDAVPLAPVTGHSSQHSLQTPGLNKTSSFSCEAHNAKGVTTSRTATITVLPQRPHHLHVVSRQPTELEVAWTPGLSGIYPLTHCNLQAVLSDDGVGIWLGKSDPPEDPLTLQVSVPPHQLRLEKLLPHTPYHIRISCSSSQGPSPWTHWLPVETTEGVPLGPPENVSAMRNGSQVLVRWQEPRVPLQGTLLGYRLAYRGQDTPEVLMDIGLTREVTLELRGDRPVANLTVSVTAYTSAGDGPWSLPVPLEPWRPGQGQPLHHLVSEPPPRAFSWPWWYVLLGALVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSDREGFPEPVVILPFMKHGDLHSFLLYSRLGDQPVFLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPVDCLDGLYALMSRCWELNPRDRPSFAELREDLENTLKALPPAQEPDEILYVNMDEGGSHLEPRGAAGGADPPTQPDPKDSCSCLTAADVHSAGRYVLCPSTAPGPTLSADRGCPAPPGQEDGA

SEQ ID NO: 116 - protein, 5 aa, synthetic construct
  REGION 1..5: note = The entire sequence of amino acids 1-5 can be repeated one or more times
GGGGS

SEQ ID NO: 117 - skipped

SEQ ID NO: 118 - protein, 6 aa, synthetic construct
  REGION 1..6: note = The entire sequence of amino acids 1-6 can be repeated one or more times
GSSGGS

SEQ ID NO: 119 - protein, 18 aa, synthetic construct
KESGSVSSEQLAQFRSLD

SEQ ID NO: 120 - protein, 14 aa, synthetic construct
EGKSSGSGSESKST

SEQ ID NO: 121 - protein, 12 aa, synthetic construct
GSAGSAAGSGEF

SEQ ID NO: 122 - protein, 5 aa, synthetic construct
  REGION 1..5: note = The entire sequence of amino acids 1-5 can be repeated one or more times
EAAAK

SEQ ID NO: 123 - protein, 12 aa, synthetic construct
CRRRRRREAEAC

SEQ ID NO: 124 - protein, 46 aa, synthetic construct
AEAAAKEAAAKEAAAKEAAAKALEAEAAAKEAAAKEAAAKEAAAKA

SEQ ID NO: 125 - protein, 8 aa, synthetic construct
GGGGGGGG

SEQ ID NO: 126 - protein, 6 aa, synthetic construct
GGGGGG

SEQ ID NO: 127 - protein, 13 aa, synthetic construct
AEAAAKEAAAAKA

SEQ ID NO: 128 - protein, 5 aa, synthetic construct
PAPAP

SEQ ID NO: 129 - protein, 17 aa, synthetic construct
VSQTSKLTRAETVFPDV

SEQ ID NO: 130 - protein, 6 aa, synthetic construct
PLGLWA

SEQ ID NO: 131 - protein, 10 aa, synthetic construct
TRHRQPRGWE

SEQ ID NO: 132 - protein, 10 aa, synthetic construct
AGNRVRRSVG

SEQ ID NO: 133 - protein, 8 aa, synthetic construct
RRRRRRRR

SEQ ID NO: 134 - protein, 4 aa, synthetic construct
GFLG

SEQ ID NO: 135 - protein, 31 aa, synthetic construct
GSSGGSGSSGGSGGGDEADGSRGSQKAGVDE

SEQ ID NO: 136 - protein, 898 aa, synthetic construct
  SIGNAL 1..30
MAPSLSPGPAALRRAPQLLLLLLAAECALADIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRGGGGSGGGGSGGGGSEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAAQGSRADYKDHDGDYKDHDIDYKDDDDK

SEQ ID NO: 137 - protein, 902 aa, synthetic construct
MAPSLSPGPAALRRAPQLLLLLLAAECALAQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSGGGGSGGGGSGGGGSSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAAQGSRADYKDHDGDYKDHDIDYKDDDDK

SEQ ID NO: 138 - protein, 714 aa, synthetic construct
  SIGNAL 1..30
MAPSLSPGPAALRRAPQLLLLLLAAECALADIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRGGGGSGGGGSGGGGSEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAAQGSRADYKDHDGDYKDHDIDYKDDDDK

SEQ ID NO: 139 - protein, 718 aa, synthetic construct
MAPSLSPGPAALRRAPQLLLLLLAAECALAQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSGGGGSGGGGSGGGGSSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAAQGSRADYKDHDGDYKDHDIDYKDDDDK

SEQ ID NO: 140 - protein, 708 aa, synthetic construct
  SIGNAL 1..30
MAPSLSPGPAALRRAPQLLLLLLAAECALADIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRGGGGSGGGGSGGGGSEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAAGSGSGSGSGSGSYPYDVPDYA

SEQ ID NO: 141 - protein, 727 aa, synthetic construct
MGWSCIILFLVATATGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECRRKRGSGEGRGSLLTCGDVEENPGPMGWSCIILFLVATATGEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 705 aa, synthetic construct
MAPSLSPGPAALRRAPQLLLLLLAAECALAALLPAREATQFLRPRQRRAFQVFEEAKQGHLERECVEELCSREEAREVFENDPETDYFYPRYLDCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAAQGSRADYKDHDGDYKDHDIDYKDDDDK

SEQ ID NO: 143 - DNA, 2697 bp, synthetic construct
  source 1..2697: mol_type = other DNA
atggccccttcgctctcgcccgggcccgccgccctgcgccgcgcgccgcagctgctgctgctgctgctggccgcggagtgcgcgcttgccgacattcagatgactcaatctcctagctctctgagcgcctccgttggagatagagtcactattacctgcagagccagccaatccatcagctcttatctaaattggtaccaacagaagcccggcaaagcgccaaagctgctcatctacgctgcaagctccttacagagcggagtacccagcagattctcaggcagtggcagtgggactgacttcacattgacgattagctctctgcagcctgaagactttgccacatactattgtcagcagagctatagcaccccgctgacgtttggaggcggaactaaggtggaaatcaagagaggaggcgggggctccggcgggggtggctcggggggaggaggctcagaggttcagcttgtcgagtctggggggggagtcgttcagccaggtagaagcctcagactgagctgtgccgcaagtgggtttgctttttcatcttacggtatgcactgggtgagacaggctcctggcaaaggactcgagtgggtcgctgtaatatggttcgatggtacaaagaaatactataccgatagtgtgaaaggaagattcaccatttcacgagacaacagtaaaaataccttgtaccttcagatgaacaccctgagagcagaagacacagccgtgtactactgcgccagagatagaggtatcggagcaaggcgtggtccctattatatggatgtgtgggggaagggaacaacagtgactgtgagctctggcgggggcggcagcggcggcggtggcagctgcatcaacaagtatgggtctccgtacaccaaaaactcaggcttcgccacctgcgtgcaaaacctgcctgaccagtgcacgcccaacccctgcgataggaaggggacccaagcctgccaggacctcatgggcaacttcttctgcctgtgtaaagctggctgggggggccggctctgcgacaaagatgtcaacgaatgcagccaggagaacgggggctgcctccagatctgccacaacaagccgggtagcttccactgttcctgccacagcggcttcgagctctcctctgatggcaggacctgccaagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcccaaggatcccgggctgactacaaagaccatgacggtgattataaagatcatgacatcgactacaaggatgacgatgacaagtga

SEQ ID NO: 144 - DNA, 2709 bp, synthetic construct
  source 1..2709: mol_type = other DNA
atggccccttcgctctcgcccgggcccgccgccctgcgccgcgcgccgcagctgctgctgctgctgctggccgcggagtgcgcgcttgcccaggttcagctggttgagagcggaggcaatctggttcagcccggtggtagtctgcgtctgtcttgtgcggcgtcagggttcactttcggtagtttttcaatgagctgggtccgtcaggcaccaggcggtgggctggaatgggtggcaggtctgtctgcacgtagctccctgacccactatgcagatagtgttaaagggcggttcacaatttcacgcgacaacgctaagaatagcgtctacctgcaaatgaactccctgcgggtcgaggataccgcagtgtattactgcgctcgccgttcttatgactctagtggatactggggccatttttatagctacatggatgtgtggggacagggcactctggtgaccgtttccggaggcggtgggtctggaggcggtgggagtggaggcggtgggtcaagcgttctgacccagccgtcctctgtcagcgccgcgccaggccagaaagtgacaatttcctgttctggaagtacttcaaacatcggcaacaattatgtttcctggtatcagcagcacccgggcaaagcgcccaagctgatgatttatgatgtgtctaaacgtccaagtggtgttcctgaccggttcagcggttccaagtctgggaatagtgcctcactggacatctcaggcctgcaaagcgaagatgaggcggactattactgcgcagcttgggatgacagcctgtccgaatttctgttcggcaccgggacaaagctgaccgtgctgggcggcgggggcggcagcggcggcggtggcagctgcatcaacaagtatgggtctccgtacaccaaaaactcaggcttcgccacctgcgtgcaaaacctgcctgaccagtgcacgcccaacccctgcgataggaaggggacccaagcctgccaggacctcatgggcaacttcttctgcctgtgtaaagctggctgggggggccggctctgcgacaaagatgtcaacgaatgcagccaggagaacgggggctgcctccagatctgccacaacaagccgggtagcttccactgttcctgccacagcggcttcgagctctcctctgatggcaggacctgccaagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcccaaggatcccgggctgactacaaagaccatgacggtgattataaagatcatgacatcgactacaaggatgacgatgacaagtga

SEQ ID NO: 145 - DNA, 2145 bp, synthetic construct
  source 1..2145: mol_type = other DNA
atggccccttcgctctcgcccgggcccgccgccctgcgccgcgcgccgcagctgctgctgctgctgctggccgcggagtgcgcgcttgccgacattcagatgactcaatctcctagctctctgagcgcctccgttggagatagagtcactattacctgcagagccagccaatccatcagctcttatctaaattggtaccaacagaagcccggcaaagcgccaaagctgctcatctacgctgcaagctccttacagagcggagtacccagcagattctcaggcagtggcagtgggactgacttcacattgacgattagctctctgcagcctgaagactttgccacatactattgtcagcagagctatagcaccccgctgacgtttggaggcggaactaaggtggaaatcaagagaggaggcgggggctccggcgggggtggctcggggggaggaggctcagaggttcagcttgtcgagtctggggggggagtcgttcagccaggtagaagcctcagactgagctgtgccgcaagtgggtttgctttttcatcttacggtatgcactgggtgagacaggctcctggcaaaggactcgagtgggtcgctgtaatatggttcgatggtacaaagaaatactataccgatagtgtgaaaggaagattcaccatttcacgagacaacagtaaaaataccttgtaccttcagatgaacaccctgagagcagaagacacagccgtgtactactgcgccagagatagaggtatcggagcaaggcgtggtccctattatatggatgtgtgggggaagggaacaacagtgactgtgagctctggcgggggcggcagcggcggcggtggcagcgacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcccaaggatcccgggctgactacaaagaccatgacggtgattataaagatcatgacatcgactacaaggatgacgatgacaagtga

SEQ ID NO: 146 - DNA, 2157 bp, synthetic construct
  source 1..2157: mol_type = other DNA
atggccccttcgctctcgcccgggcccgccgccctgcgccgcgcgccgcagctgctgctgctgctgctggccgcggagtgcgcgcttgcccaggttcagctggttgagagcggaggcaatctggttcagcccggtggtagtctgcgtctgtcttgtgcggcgtcagggttcactttcggtagtttttcaatgagctgggtccgtcaggcaccaggcggtgggctggaatgggtggcaggtctgtctgcacgtagctccctgacccactatgcagatagtgttaaagggcggttcacaatttcacgcgacaacgctaagaatagcgtctacctgcaaatgaactccctgcgggtcgaggataccgcagtgtattactgcgctcgccgttcttatgactctagtggatactggggccatttttatagctacatggatgtgtggggacagggcactctggtgaccgtttccggaggcggtgggtctggaggcggtgggagtggaggcggtgggtcaagcgttctgacccagccgtcctctgtcagcgccgcgccaggccagaaagtgacaatttcctgttctggaagtacttcaaacatcggcaacaattatgtttcctggtatcagcagcacccgggcaaagcgcccaagctgatgatttatgatgtgtctaaacgtccaagtggtgttcctgaccggttcagcggttccaagtctgggaatagtgcctcactggacatctcaggcctgcaaagcgaagatgaggcggactattactgcgcagcttgggatgacagcctgtccgaatttctgttcggcaccgggacaaagctgaccgtgctgggcggcgggggcggcagcggcggcggtggcagcgacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctcaccgtgggaggtattcccttccatgagaaggacctcgtgcagcctataaaccctcgtctggatggctgtatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgccggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcccaaggatcccgggctgactacaaagaccatgacggtgattataaagatcatgacatcgactacaaggatgacgatgacaagtga

SEQ ID NO: 147 - DNA, 2127 bp, synthetic construct
  source 1..2127: mol_type = other DNA
atggccccttcgctctcgcccgggcccgccgccctgcgccgcgcgccgcagctgctgctgctgctgctggccgcggagtgcgcgcttgccgacattcagatgactcaatctcctagctctctgagcgcctccgttggagatagagtcactattacctgcagagccagccaatccatcagctcttatctaaattggtaccaacagaagcccggcaaagcgccaaagctgctcatctacgctgcaagctccttacagagcggagtacccagcagattctcaggcagtggcagtgggactgacttcacattgacgattagctctctgcagcctgaagactttgccacatactattgtcagcagagctatagcaccccgctgacgtttggaggcggaactaaggtggaaatcaagagaggaggcgggggctccggcgggggtggctcggggggaggaggctcagaggttcagcttgtcgagtctggggggggagtcgttcagccaggtagaagcctcagactgagctgtgccgcaagtgggtttgctttttcatcttacggtatgcactgggtgagacaggctcctggcaaaggactcgagtgggtcgctgtaatatggttcgatggtacaaagaaatactataccgatagtgtgaaaggaagattcaccatttcacgagacaacagtaaaaataccttgtaccttcagatgaacaccctgagagcagaagacacagccgtgtactactgcgccagagatagaggtatcggagcaaggcgtggtccctattatatggatgtgtgggggaagggaacaacagtgactgtgagctctggcgggggcggcagcggcggcggtggcagcgacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagccggcagcggcagcggcagcggcagcggcagcggcagctacccatacgatgttccagattacgcttga

SEQ ID NO: 148 - DNA, 2190 bp, synthetic construct
  source 1..2190: mol_type = other DNA
ggatccatgggctggtcctgcatcatcctgttcctggtggccaccgccaccggcgacattcagatgactcaatctcctagctctctgagcgcctccgttggagatagagtcactattacctgcagagccagccaatccatcagctcttatctaaattggtaccaacagaagcccggcaaagcgccaaagctgctcatctacgctgcaagctccttacagagcggagtacccagcagattctcaggcagtggcagtgggactgacttcacattgacgattagctctctgcagcctgaagactttgccacatactattgtcagcagagctatagcaccccgctgacgtttggaggcggaactaaggtggaaatcaagagaaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgtcgcagaaaacgcggaagcggagagggcagaggaagtcttctaacatgcggtgacgtggaggagaatcccggccctatgggctggtcctgcatcatcctgttcctggtggccaccgccaccggcgaggttcagcttgtcgagtctggggggggagtcgttcagccaggtagaagcctcagactgagctgtgccgcaagtgggtttgctttttcatcttacggtatgcactgggtgagacaggctcctggcaaaggactcgagtgggtcgctgtaatatggttcgatggtacaaagaaatactataccgatagtgtgaaaggaagattcaccatttcacgagacaacagtaaaaataccttgtaccttcagatgaacaccctgagagcagaagacacagccgtgtactactgcgccagagatagaggtatcggagcaaggcgtggtccctattatatggatgtgtgggggaagggaacaacagtgactgtgagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatctagcgacaaaactcacacaagcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 149 - DNA, 2118 bp, synthetic construct
  source 1..2118: mol_type = other DNA
atggccccttcgctctcgcccgggcccgccgccctgcgccgcgcgccgcagctgctgctgctgctgctggccgcggagtgcgcgcttgccgcgctgttgccggcgcgcgaggccacgcagttcctgcggcccaggcagcgccgcgcctttcaggtcttcgaggaggccaagcagggccacctggagagggagtgcgtggaggagctgtgcagccgcgaggaggcgcgggaggtgttcgagaacgaccccgagacggattatttttacccaagatacttagactgcatcaacaagtatgggtctccgtacaccaaaaactcaggcttcgccacctgcgtgcaaaacctgcctgaccagtgcacgcccaacccctgcgataggaaggggacccaagcctgccaggacctcatgggcaacttcttctgcctgtgtaaagctggctgggggggccggctctgcgacaaagatgtcaacgaatgcagccaggagaacgggggctgcctccagatctgccacaacaagccgggtagcttccactgttcctgccacagcggcttcgagctctcctctgatggcaggacctgccaagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcccaaggatcccgggctgactacaaagaccatgacggtgattataaagatcatgacatcgactacaaggatgacgatgacaagtga

SEQ ID NO: 150 - protein, 961 aa, synthetic construct
  SIGNAL 1..30
MAPSLSPGPAALRRAPQLLLLLLAAECALADDVLTQTPLSLPVTPGQPASISCRSSQSIVHSNGNTYLEWYLQKPGQSPQLLIYKVSNRFSGVPDRFSGSGSGTDFTLKISRVEAEDVGVYYCFQGSLVPWTFGQGTKVEIKGGGGSGGGGSGGGGSEVQLVESGGGLVQPGGSLRLSCAASGLIFRSYGMSWVRQAPGKGLEWVATINSGGTYTYYPDSVKGRFTISRDNSKNTLYLQMNSLRAEDTAVYYCANSYSGAMDYWGQGTLVTVSSGGGGSGGGGSDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAAGSGSGSGSGSGSYPYDVPDYAEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 151 - DNA, 2886 bp, synthetic construct
  source 1..2886: mol_type = other DNA
atggccccttcgctctcgcccgggcccgccgccctgcgccgcgcgccgcagctgctgctgctgctgctggccgcggagtgcgcgcttgccgacgatgtattaacacaaactcccctatcattgccggtgaccccgggccaaccagcttcgatcagctgccgtagctctcagagcatcgtgcacagcaacggtaatacctacctggaatggtatttgcaaaaaccgggtcaatccccgcagttgctgatttataaagtttcgaatcgtttcagcggtgttccggatcgtttcagcggctctggctccggcaccgattttacgctgaagatcagtcgcgtggaagcggaggacgtgggtgtctactactgctttcagggtagtttggtgccgtggacctttggtcagggtactaaggtggaaattaagggtggtgggggatcaggtggcggcggcagcggcggtggcgggagcgaggtacaactagttgaatcaggtggagggttggttcagccaggtggttcgctgcgtctgagttgtgcggcaagcggtttgatctttcgcagctatggtatgagctgggttcgtcaggcgccgggcaagggtctggagtgggtggcgaccattaactctggcggcacgtacacctactatcccgactccgtgaaaggccgtttcaccatctcccgcgacaatagcaaaaacaccctgtatttgcagatgaactcgctccgcgcagaggacaccgctgtgtactactgcgccaattcctacagcggtgctatggattattggggtcagggcacattggtgactgtaagcagcggcgggggcggcagcggcggcggtggcagcgacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagccggcagcggcagcggcagcggcagcggcagcggcagctacccatacgatgttccagattacgctgagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 152 - protein, 953 aa, synthetic construct
  SIGNAL 1..30
MAPSLSPGPAALRRAPQLLLLLLAAECALASYELTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVLGGGGSGGGGSGGGGSEVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSSGGGGSGGGGSDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAAGSGSGSGSGSGSYPYDVPDYAEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 153 - DNA, 2862 bp, synthetic construct
  source 1..2862: mol_type = other DNA
atggccccttcgctctcgcccgggcccgccgccctgcgccgcgcgccgcagctgctgctgctgctgctggccgcggagtgcgcgcttgcctcctatgagctgactcagccaccctcggtgtcagtgtccccaggacagacggccaggatcacctgctctggagaagcattgccaatgcaatttgctcattggtaccaacagaggccaggcaaggccccagtgatagtggtgtacaaagacagtgagagaccgtcaggtgtccctgagcgattctctggctccagctcagggacaacagccacgttgaccatcactggagtccaggcagaagatgaggctgactattactgccagtcgccagacagcactaacacttatgaagtcttcggcggagggaccaagctgaccgtcctaggtggtgggggatcaggtggcggcggcagcggcggtggcgggagcgaggtgcagctggtggagtctgggggaggtctggtcgagccgggggggtccctaagactctcctgtgcagtctccggattcgatttcgaaaaagcctggatgagttgggtccgccaggctccagggcaggggctacagtgggttgcccgtatcaagagcacagctgatggtgggacaacaagctacgccgcccccgtggaaggcaggttcatcatctcaagagatgattcgagaaacatgctttatctgcaaatgaacagtctgaaaactgaagacacagccgtctattattgtacatcagcccactggggccagggaaccctggtcaccgtctcctcgggcgggggcggcagcggcggcggtggcagcgacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagccggcagcggcagcggcagcggcagcggcagcggcagctacccatacgatgttccagattacgctgagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 154 - protein, 701 aa, synthetic construct
  SIGNAL 1..24
MRVLGGRCGALLACLLLVLPVSEADIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRGGGGSGGGGSGGGGSEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSVVSVCLPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNSQGSRADYKDHDGDYKDHDIDYKDDDDK

SEQ ID NO: 155 - DNA, 2106 bp, synthetic construct
  source 1..2106: mol_type = other DNA
atgagggtcctgggtgggcgctgcggggcgctgctggcgtgtctcctcctagtgcttcccgtctcagaggcagacattcagatgactcaatctcctagctctctgagcgcctccgttggagatagagtcactattacctgcagagccagccaatccatcagctcttatctaaattggtaccaacagaagcccggcaaagcgccaaagctgctcatctacgctgcaagctccttacagagcggagtacccagcagattctcaggcagtggcagtgggactgacttcacattgacgattagctctctgcagcctgaagactttgccacatactattgtcagcagagctatagcaccccgctgacgtttggaggcggaactaaggtggaaatcaagagaggaggcgggggctccggcgggggtggctcggggggaggaggctcagaggttcagcttgtcgagtctggggggggagtcgttcagccaggtagaagcctcagactgagctgtgccgcaagtgggtttgctttttcatcttacggtatgcactgggtgagacaggctcctggcaaaggactcgagtgggtcgctgtaatatggttcgatggtacaaagaaatactataccgatagtgtgaaaggaagattcaccatttcacgagacaacagtaaaaataccttgtaccttcagatgaacaccctgagagcagaagacacagccgtgtactactgcgccagagatagaggtatcggagcaaggcgtggtccctattatatggatgtgtgggggaagggaacaacagtgactgtgagctctggcgggggcggcagcggcggcggtggcagcgttgtttcagtgtgccttcccttgaaccttgacacaaagtatgaattactttacttggcggagcagtttgcaggggttgttttatatttaaaatttcgtttgccagaaatcagcagattttcagcagaatttgatttccggacatatgattcagaaggcgtgatactgtacgcagaatctatcgatcactcagcgtggctcctgattgcacttcgtggtggaaagattgaagttcagcttaagaatgaacatacatccaaaatcacaactggaggtgatgttattaataatggtctatggaatatggtgtctgtggaagaattagaacatagtattagcattaaaatagctaaagaagctgtgatggatataaataaacctggacccctttttaagccggaaaatggattgctggaaaccaaagtatactttgcaggattccctcggaaagtggaaagtgaactcattaaaccgattaaccctcgtctagatggatgtatacgaagctggaatttgatgaagcaaggagcttctggaataaaggaaattattcaagaaaaacaaaataagcattgcctggttactgtggagaagggctcctactatcctggttctggaattgctcaatttcacatagattataataatgtatccagtgctgagggttggcatgtaaatgtgaccttgaatattcgtccatccacgggcactggtgttatgcttgccttggtttctggtaacaacacagtgccctttgctgtgtccttggtggactccacctctgaaaaatcacaggatattctgttatctgttgaaaatactgtaatatatcggatacaggccctaagtctatgttccgatcaacaatctcatctggaatttagagtcaacagaaacaatctggagttgtcgacaccacttaaaatagaaaccatctcccatgaagaccttcaaagacaacttgccgtcttggacaaagcaatgaaagcaaaagtggccacatacctgggtggccttccagatgttccattcagtgccacaccagtgaatgccttttataatggctgcatggaagtgaatattaatggtgtacagttggatctggatgaagccatttctaaacataatgatattagagctcactcatgtccatcagtttggaaaaagacaaagaattctcaaggatcccgggctgactacaaagaccatgacggtgattataaagatcatgacatcgactacaaggatgacgatgacaagtga

SEQ ID NO: 156 - protein, 684 aa, synthetic construct
  SIGNAL 1..21
METDTLLLWVLLLWVPGSTGDEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGGGGSGGGGSDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAADYKDHDGDYKDHDIDYKDDDDK

SEQ ID NO: 157 - DNA, 2055 bp, synthetic construct
  source 1..2055: mol_type = other DNA
atggagacagacacactcctgctatgggtactgctgctctgggttccaggttccactggtgacgaggttcagcttgtcgagtctggggggggagtcgttcagccaggtagaagcctcagactgagctgtgccgcaagtgggtttgctttttcatcttacggtatgcactgggtgagacaggctcctggcaaaggactcgagtgggtcgctgtaatatggttcgatggtacaaagaaatactataccgatagtgtgaaaggaagattcaccatttcacgagacaacagtaaaaataccttgtaccttcagatgaacaccctgagagcagaagacacagccgtgtactactgcgccagagatagaggtatcggagcaaggcgtggtccctattatatggatgtgtgggggaagggaacaacagtgactgtgagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacggcggaggtggaagcggaggcggtggaagcgacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctcaccgtgggaggtattcccttccatgagaaggacctcgtgcagcctataaaccctcgtctggatggctgtatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgccggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagccgactacaaagaccatgacggtgattataaagatcatgacatcgactacaaggatgacgatgacaagtga

SEQ ID NO: 158 - protein, 907 aa, synthetic construct
  SIGNAL 1..21
METDTLLLWVLLLWVPGSTGDEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAADYKDHDGDYKDHDIDYKDDDDK

SEQ ID NO: 159 - DNA, 2724 bp, synthetic construct
  source 1..2724: mol_type = other DNA
atggagacagacacactcctgctatgggtactgctgctctgggttccaggttccactggtgacgaggttcagcttgtcgagtctggggggggagtcgttcagccaggtagaagcctcagactgagctgtgccgcaagtgggtttgctttttcatcttacggtatgcactgggtgagacaggctcctggcaaaggactcgagtgggtcgctgtaatatggttcgatggtacaaagaaatactataccgatagtgtgaaaggaagattcaccatttcacgagacaacagtaaaaataccttgtaccttcagatgaacaccctgagagcagaagacacagccgtgtactactgcgccagagatagaggtatcggagcaaggcgtggtccctattatatggatgtgtgggggaagggaacaacagtgactgtgagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaaggcggaggtggaagcggaggcggtggaagcgacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctcaccgtgggaggtattcccttccatgagaaggacctcgtgcagcctataaaccctcgtctggatggctgtatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgccggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagccgactacaaagaccatgacggtgattataaagatcatgacatcgactacaaggatgacgatgacaagtga

SEQ ID NO: 160 - protein, 5 aa, synthetic construct
GGGGS

SEQ ID NO: 161 - protein, 232 aa, synthetic construct
  SIGNAL 1..16
MGWSCIILFLVATATGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 914 aa, synthetic construct
  SIGNAL 1..21
METDTLLLWVLLLWVPGSTGDDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTFPPEQEEMTKNQVSLRCLVKGFYPSDIAVEWESNGQPENNYKTTKPVLDSDGSFRLESRLTVDKSRWQEGNVFSCSVMHEACSWHLCKSLSLSLGKGGGGSGGGGSGGGGSDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAAHHHHHH

SEQ ID NO: 163 - protein, 925 aa, synthetic construct
  SIGNAL 1..21
METDTLLLWVLLLWVPGSTGDDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGKGGGGSGGGGSGGGGSDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAAHHHHHH

SEQ ID NO: 164 - protein, 504 aa, synthetic construct
  SIGNAL 1..21
METDTLLLWVLLLWVPGSTGDDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 165 - protein, 925 aa, synthetic construct
  SIGNAL 1..21
METDTLLLWVLLLWVPGSTGDDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAAHHHHHH

SEQ ID NO: 166 - protein, 730 aa, synthetic construct
METDTLLLWVLLLWVPGSTGDEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVKLVESGGDLVKPGGSLKLSCAASGFTFSSYGMSWVRQTPDKRLEWVATISSGGSYTYYPDSVKGRFTISRDNAKNTLYLQMSSLKSEDTAMYYCARHPIYYTYDDTMDYWGQGTSVTVSSGGGGSGGGGSGGGGSDIVLTQSPAIMAASPGEKVTMTCSASSSVSSGNFHWYQQKPGTSPKLWIYRTSNLASGVPARFSGSGSGTSYSLTISSMEAEDAATYYCQQWSGYPWTFGGGTKLEIK

SEQ ID NO: 167 - protein, 730 aa, synthetic construct; identical to SEQ ID NO: 166

SEQ ID NO: 168 - protein, 730 aa, synthetic construct
METDTLLLWVLLLWVPGSTGDEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKGGGGSGGGGSEVKLVESGGDLVKPGGSLKLSCAASGFTFSSYGMSWVRQTPDKRLEWVATISSGGSYTYYPDSVKGRFTISRDNAKNTLYLQMSSLKSEDTAMYYCARHPIYYTYDDTMDYWGQGTSVTVSSGGGGSGGGGSGGGGSDIVLTQSPAIMAASPGEKVTMTCSASSSVSSGNFHWYQQKPGTSPKLWIYRTSNLASGVPARFSGSGSGTSYSLTISSMEAEDAATYYCQQWSGYPWTFGGGTKLEIK

SEQ ID NO: 169 - protein, 475 aa, synthetic construct
METDTLLLWVLLLWVPGSTGDEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK